(19) United States
(12) Patent Application Publication
(10) Pub. No.: US 2012/0269728 A1
(43) Pub. Date: Oct. 25, 2012

(54) Methods and compositions for detecting one or more target agents using tracking components

(75) Inventors: I-Min M. Jen, Sunnyvale, CA (US); Mark O. Trulson, San Jose, CA (US); Kilian Dill, La Honda, CA (US); Marc R. Labgold, Reston, VA (US); Dawei Sheng, Mountain View, CA (US); George G. Jokhadze, Mountain View, CA (US); Chandramohan V. Ammini, Mountain View, CA (US); Peter Lobban, Los Altos Hills, CA (US)

(73) Assignee: Antara BioSciences Inc., Mountain View, CA (US)

(21) Appl. No.: 11/812,691

(22) Filed: Jun. 21, 2007

Related U.S. Application Data
(63) Continuation of application No. 11/703,103, filed on Feb. 7, 2007.
(60) Provisional application No. 60/851,697, filed on Oct. 13, 2006.
(60) Provisional application No. 60/853,697, filed on Oct. 23, 2006.
(60) Provisional application No. 60/859,441, filed on Nov. 16, 2006.
(60) Provisional application No. 60/874,291, filed on Dec. 12, 2006.
(60) Provisional application No. 60/876,279, filed on Dec. 21, 2006.
(60) Provisional application No. 60/834,951, filed on Aug. 2, 2006.

(51) Int. Cl. G01N 23/00; G01N 33/53; G01N 21/76; G01N 33/567; G01N 27/72; A61K 49/00; A61B 5/07; C12Q 1/68; G01N 33/569; G01N 30/02

(52) U.S. Cl. 424/9.1; 435/7.1; 436/501; 436/94; 436/86; 600/302; 435/6.11; 435/7.21

(57) ABSTRACT

The present invention provides devices and methods for real time detection of target agents in a sample. These devices utilize tracking technology and selective binding to allow the identification of one or more target agents in a sample, and preferably in a biological sample. The present invention provides specific embodiments employing radio frequency identification devices.

### CROSS-REFERENCE TO RELATED APPLICATIONS

This application claims the benefit of U.S. Provisional Patent Application Ser. No. 60/834,951, filed Aug. 2, 2006, currently pending; U.S. Provisional Patent Application Ser. No. 60/851,697, filed Oct. 13, 2006, currently pending; U.S. Provisional Patent Application Ser. No. 60/853,697, filed Oct. 23, 2006, currently pending; U.S. Provisional Patent Application Ser. No. 60/859,441, filed Nov. 16, 2006, currently pending; U.S. Provisional Patent Application Ser. No. 60/874,291, filed Dec. 12, 2006, currently pending; U.S. Provisional Patent Application Ser. No. 60/876,279, filed Dec. 21, 2006, currently pending; and U.S. patent application Ser. No. 11/703,103, filed Feb. 7, 2007, currently pending, all of which are herein incorporated by reference in their entireties for all purposes.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Radio Frequency Identification (RFID) is an automatic identification method, relying on storing and remotely retrieving data using devices called RFID tags or transponders. Commonly used RFID tags include objects that can be attached to or incorporated into a product, animal, or person for the purpose of identification using radio waves. The tags are the backbone of the technology and come in various shapes, sizes and read ranges including thin and flexible “smart labels” which can be laminated between paper or plastic. Chip-based RFID tags contain silicon chips and, in some embodiments, antennas. Passive tags require no internal power source, whereas active tags require an internal power source.

RFID systems typically consist of a number of components including tags, handheld or stationary readers, data input units, and system software. RFID provides an automated (or automatable) way to collect information about a product, place, time or transaction quickly, easily and without human error. It provides a contact-less data link, without need for line-of-sight or concerns about harsh or dirty environments that restrict other automatic ID technologies such as bar codes. In addition, RFID can provide more than just an identification device. An RFID tag can be used as a data carrier, and information can be written to and updated on the tag in real time.

RFID has been applied to a variety of applications in varied industries. Today, RFID is used for such applications as vehicle and personnel access control, automotive security (e.g., anti-theft) systems, product and asset tracking, and supply chain automation. Additional applications include payment and loyalty management, sports timing, livestock identification, and document management.

In one common application, RFID is used in conjunction with a gas-station payment system, employing radio frequency signals to enable two-way, wireless communication between a key ring tag and a gasoline pump or counter-top reader. The desired purchase is electronically charged to a gas-station customer's credit card of choice without the presentation of the credit card or the participation of an attendant.

Biological assays typically involve large numbers of compounds, thus current screening assays can be expensive and time-consuming. In certain assays, radiolabeling of reference compounds have been used; however, these assays are expensive and require complicated disposal protocols and dedicated laboratory areas due to the use of radioactive materials. In other screening assays, fluorescently-labeled materials have been used, but such assays suffer from the occurrence of false positives, difficulty in detection of the fluorescent signal, and the denaturation of the fluorescent compound during handling. Both radiolabeling and fluorescence assays are subject to the limited number of unique identifiers available for identification, given the fact that only a limited number of different radiolabels and fluorescent compounds are commercially available.

### SUMMARY OF THE INVENTION

Accurate and rapid assays for biological materials are desirable. The present invention provides methods and compositions for such assays. Methods for detecting one or more target agents in a sample are taught. In preferred embodiments, target agents in the sample are “captured” by a capture moiety conjugated to a tracking component, wherein the tracking component serves as a proxy for presence of the target agent in the sample, for example, by being identified by a tracking component detector. In some embodiments, a signal from the tracking component is affected by a moiety in association with a target agent. In certain embodiments, the tracking components are RFID tags and the tracking component detectors are RFID readers. The RFID tags employed in the methods herein can be of many shapes and sizes, and may be coated or encapsulated, but preferably encode information that enables rapid and accurate determination of the presence of a target agent in a sample. Such methods typically allow for rapid and accurate detection without the need for purification and/or amplification steps. Further, such methods can allow for determination of the presence or sequence of a target nucleic acid, genotype of a SNP, or presence of a nucleic acid mutation. In certain embodiments, the methods are performed in vivo, for example, to detect target agents including but not limited to cancer markers, genetic mutations or other nucleic acid sequences, proteins, metabolites, toxins, drugs, pathogens, microorganisms, or viruses. In certain embodiments, the detection of target agents comprises electrochemical, fluorescent, magnetic, or other detection methods known in the art. Further, embodiments are not limited to the description listed within the Brief Summary and may include other embodiments and limitations from other parts of the specification.

It is an object of the present invention to provide an RFID device, an RFID system and method for tracking results, and use and/or manufacture of RFID diagnostic devices. It is an object of the present invention to provide RFID devices, systems and methods for “labeling” RFID devices with useful information including, inter alia, unique identifiers, dates of manufacture, lot numbers, sites of manufacture, operators, technicians, sources of materials, etc.

Also provided is a method of determining a presence of a target agent in a sample wherein the target agent is not contacted with a detection device comprising mixing the sample with tracking complexes that include a tracking component and a capture moiety that specifically binds to the target agent. The tracking complexes bound to the target agents (“reacted complexes”) are separated from tracking complexes that did not bind to the target agents. The reacted complexes are treated so as to remove the target agents, and are subsequently subjected to interrogation by a detection device. A signal detected from the tracking components is indicative of the presence of a target agent in the sample. In some embodiments, the tracking component is an RFID tag. In some embodiments, the tracking component is conjugated to a plurality of capture moieties. In specific embodiments, chromatography and/or immobilized binding partners are used to separate tracking complexes that bind to the target agents from those that do not. In certain embodiments, a plurality of different target agents is detected simultaneously. In some embodiments, the signal detected when the target agent is in the sample is altered relative to a baseline signal detected when the target agent is not in the sample.

In certain embodiments, the present invention provides RFID-based methods for real-time detection of target agents in a sample. These RFID-based methods utilize tracking technology and selective binding to allow the identification of one or more target agents in a sample. In certain embodiments, an RFID-based method comprises use of an RFID device (e.g., an RFID complex), which comprises, in varied orders or combinations a) a tracking component; and b) a plurality of capture moieties. In some such embodiments, an RFID device further comprises a polymer that is uniformly distributed on at least one surface of a tracking component. In certain embodiments, an RFID device comprises, in varied orders or combinations: a) a tracking component; b) a matrix affixed to, embedded in, and/or associated with the tracking component; c) a polymer that is uniformly distributed on at least one surface of the matrix; d) and a plurality of capture moieties. In certain embodiments, capture moieties of an RFID device preferentially bind to a specific target agent, which is, for example, useful when attempting to detect and quantify very low levels of the target agent in a sample.

A polymer distributed on a tracking component (or matrix) may, e.g., facilitate the conjugation of the tracking component (e.g., via a matrix) to a capture moiety for detection of a target agent in a sample. In specific embodiments, the RFID device also comprises an adapter molecule (e.g., a coupling agent such as avidin or strepavidin) associated with such a polymer. Adaptor molecules may be conjugated directly to a polymer or via a linker, e.g. a peptidic spacer. In certain embodiments, a polymer used in an RFID device is a biocompatible polymer. Examples of such polymers include, but are not limited to, polytetrafluoroethylene (PTFE), Sephadex™, polystyrene, polyethylene, and polypropylene.

In some embodiments, the present invention utilizes surface chemistries adapted for multiplexed analysis of a sample, e.g., conjugation of multiple different capture moieties to an RFID device, and/or the use of tracking components (e.g., RFID tags) with unique identification numbers to allow simultaneous detection of multiple target agents. This can be particularly helpful to reduce the time and cost of identification of multiple target agents in a sample. Optionally, if a positive result is obtained using multiplexed analysis, presence of a particular target agent may be further confirmed or quantified by subsequent use of RFID devices comprising capture moieties specific for only the particular target agent.

In further embodiments, the RFID devices of the invention may be attached, either directly or indirectly (e.g., through a target capture event) in a known orientation to a second, planar matrix to form an RFID device array. In related embodiments, the RFID devices of the invention may be attached, either directly or indirectly (e.g., through a target capture event) in a random orientation to a second, planar matrix to form an RFID device array.

In certain embodiments, the devices and methods of the invention comprise RFID devices that are a part of an integrated diagnostic system. For example, the RFID devices of the present invention can be integrated into a fluorescent, chemiluminescent, colorimetric, or electrochemical detection diagnostic system to perform high-sensitivity identification and quantification of a target agent in a sample. It is one object of the present invention to use RFID devices and methods of the invention with integrated RFID systems; it is another object of the invention to use RFID devices and methods of the invention with local RFID systems.

In further embodiments, the method comprises generating control signals from a computer-implemented system to cause a radio frequency interface to retrieve data from an RFID device, and in a distinct further embodiment the method comprises generating control signals by a computer-implemented system to transmit data to an RFID device via a radio frequency interface. In some embodiments, a signal from the RFID device is affected by a moiety in association with a target agent.

In certain embodiments, a computer-implemented system is an integrated system comprising a 3-tier architecture having a web browser, a web server program, and a database server, and further comprises a client-side application that controls operation of a radio frequency interface. In certain further embodiments, the computer-implemented system comprises a USB interface between the web browser and an RFID reader. In another related embodiment, the computer-implemented system comprises a 2-tier architecture having a program (e.g., a macro) on a client side and a database server. In some embodiments, the computer-implemented system comprises a 2-tier architecture having a stand-alone client application and a database server in communication with the client application. In certain further embodiments, the client application is a compiled application.

In specific embodiments, selective binding of a capture moiety associated with an RFID device is used for detection and identification of target agents in a sample, and/or for the detection and identification of binding events between molecules or compounds. Such selective binding includes, but is not limited to, binding events between nucleic acids, receptors and agonists, receptors and antagonists, enzymes and substrates, enzymes and inhibitors, antibodies and proteins, antibodies and antigens, and peptidomimetic molecules and proteins. In some embodiments, a signal from the RFID device is affected by binding of the capture moiety to the target agent.

In some embodiments of the invention, a method is provided to determine the presence of a target agent in a sample comprising introducing the sample to a matrix comprising one or more immobilized binding partners that bind to and immobilize the target agent. The immobilized target agent is exposed to tracking complexes that include a tracking component and a capture moiety that binds to the target agent. Tracking complexes that did not bind target agent are removed and tracking complexes that are immobilized on the matrix by virtue of their interaction with the target agent are subjected to interrogation by a detection device. A signal detected from the tracking components is indicative of the presence of a target agent in the sample. In certain embodiments, the signal that indicates the presence of a target agent in the sample is altered relative to a baseline signal. In some embodiments, the tracking component is an RFID tag. In some embodiments, the tracking component is conjugated to a plurality of capture moieties. In certain embodiments, a plurality of different target agents is detected simultaneously.

One aspect of the invention provides a method for using a tracking complex that is an RFID device with an associated target agent in the detection of a binding event between the target agent and an immobilized binding partner. This embodiment includes the use of (1) an RFID tag associated with a target agent (“loaded RFID-target agent complex”), and (2) an immobilized binding partner. The method includes mixing a solution containing the loaded RFID-target agent complex with the immobilized binding partner. If the loaded RFID-target agent complex binds to the immobilized binding partner, it is immobilized thereby forming immobilized loaded RFID-target agent complex; if the loaded RFID-target agent complex does not bind to the immobilized binding partner, it remains in solution. The solution phase is separated from the immobilized phase, and the immobilized phase is scanned with an RFID reader, where the RFID tags of the immobilized loaded RFID-target agent complexes are interrogated. The presence of an immobilized RFID tag indicates a binding event between the loaded RFID-target agent complex and the immobilized binding partner. The absence of an immobilized RFID tag indicates that the target agent did not bind to the immobilized binding partner.

In certain embodiments, the methods may be multiplexed, for example, by providing a plurality of immobilized binding partners to which the target agent can bind at known locations on a matrix. The location at which an RFID tag is immobilized on the matrix indicates to which immobilized binding partner the target agent bound. The method may also be multiplexed by providing a plurality of target agents, each conjugated to a different RFID tag, where information encoded within each RFID tag identifies the target agent conjugated thereto. Therefore, a particular immobilized RFID tag identifies a particular target agent bound to a binding partner. Similarly, a plurality of different immobilized binding partners and a plurality of different RFID-tagged target agents may be used in combination to detect binding events between one or more of the binding partners and one or more of the target agents.

In certain embodiments of the invention, a method is provided to determine the presence of a target agent in a sample comprising mixing the sample with tracking complexes that include a tracking component and a capture moiety that binds to the target agent. The mixture is contacted with immobilized binding partners that facilitate separation of tracking complexes that bound to target agent from tracking complexes that did not bind to target agent. Tracking complexes that did bind to target agent are exposed to a detection device that detects the tracking components of the tracking complexes. A signal detected from the tracking components is indicative of the presence of a target agent in the sample. In certain embodiments, the signal that indicates the presence of a target agent in the sample is altered relative to a baseline signal. In some embodiments, the tracking component is an RFID tag. In some embodiments, the tracking component is conjugated to a plurality of capture moieties. In some embodiments, the immobilized binding partners bind and immobilize tracking complexes that have bound to target agent, and in other embodiments the immobilized binding partners bind and immobilize tracking complexes that have not bound to target agent. In certain embodiments, a plurality of different target agents is detected simultaneously. In some embodiments, a signal from the tracking component is affected by binding of a) the capture moiety to the target agent, b) the immobilized binding partner to the tracking complexes that have bound to target agent, and/or c) the immobilized binding partner to the tracking complexes that have not bound to target agent.

Another aspect of the invention provides a method for using a tracking complex that is an RFID device with an associated capture moiety in the detection of a binding event between the capture moiety and a target agent. Certain embodiments include use of (1) an RFID device where the RFID device is associated with a capture moiety specific for a target agent (“loaded RFID complex”), and (2) an immobilized binding partner specific for a different portion of the target agent than that bound by the capture moiety to allow simultaneous association of the target agent with both the capture moiety and the immobilized binding partner. The method includes mixing the loaded RFID complex with a sample containing (or suspected of containing) the target agent to create a first mixture, wherein loaded RFID complexes that bind to the target agent are termed “reacted loaded RFID complexes.” The first mixture is mixed with the immobilized binding partner under conditions to facilitate binding of the target agent to the immobilized binding partner without disrupting the association between the target agent and the capture moiety, thereby immobilizing the reacted loaded RFID complexes and leaving unreacted loaded RFID complexes in solution. The solution phase is separated from the immobilized phase, and the immobilized phase is scanned with an RFID reader, where the identification numbers of the reacted loaded RFID complexes can be interrogated. The presence of a reacted loaded RFID complex indicates a binding event between a loaded RFID complex and a target agent, thereby confirming the presence of the target agent in the sample and the binding of the target agent to both the capture moiety and the immobilized binding partner. In related embodiments, the binding partner is specific for the target agent/capture moiety complex rather than a different portion of the target agent than that bound by the capture moiety, and in such embodiments target agent that is not bound by a capture moiety does not bind to the immobilized binding partner. In further related embodiments, the binding partner is specific for capture moieties that have not bound target agent and, as such, immobilize the unreacted loaded RFID complexes rather than the reacted loaded RFID complexes as described above. In such embodiments, the solution phase comprising the reacted loaded RFID complexes is subjected to RFID interrogation; the presence of a reacted loaded RFID complex in the solution phase indicates a binding event between that reacted loaded RFID complex and a target agent, and further confirms the presence of the target agent in the sample.

In another aspect of the present invention, a multiplexed method is provided for using a plurality of RFID devices with associated capture moieties in the detection of binding events between a plurality of target agents and the capture moieties specific therefore. Certain embodiments include use of (1) a plurality of RFID devices, each of which is associated with one or more capture moieties specific for a single target agent (“loaded RFID complexes”), where at least one loaded RFID complex exists for each target agent to be detected, and (2) a plurality of immobilized binding partners, each of which is specific for a different portion of a target agent than that bound by the capture moiety specific therefor (which allows simultaneous association of the target agents with both the capture moieties and the immobilized binding partners), where at least one immobilized binding partner exists for each target agent to be detected, and further where the plurality of immobilized binding partners are at known locations on a matrix. The method includes mixing the loaded RFID complexes with a sample containing (or suspected of containing) the target agents to create a first mixture, wherein loaded RFID complexes that bind to a target agent are termed “reacted loaded RFID complexes.” The first mixture is mixed with the immobilized binding partners under conditions to facilitate binding of the target agents to their corresponding immobilized binding partners without disrupting the association between the target agent and the capture moiety, thereby immobilizing the reacted loaded RFID complexes and leaving unreacted loaded RFID complexes in solution. The solution phase is separated from the immobilized phase, and the immobilized phase is scanned with an RFID reader, where the RFID tags in the reacted loaded RFID complexes can be interrogated, and their location on the matrix correlated to the immobilized binding partner at that location. The presence of a reacted loaded RFID complex at a particular location on the matrix indicates (i) a binding event between the particular capture moiety in the reacted loaded RFID complex and a particular target agent, (ii) a binding event between the particular immobilized binding partner at that position on the matrix and the particular target agent, and (iii) the presence of the particular target agent in the sample.

In related embodiments, a binding partner is specific for a target agent/capture moiety complex rather than a different portion of a target agent than that bound by the capture moiety, and in such embodiments target agent that is not bound by a capture moiety does not bind to the immobilized binding partner. In further related embodiments, the binding partners are specific for capture moieties that have not bound target agent and, as such, immobilize the unreacted loaded RFID complexes rather than the reacted loaded RFID complexes as described above. In such embodiments, the solution phase comprising the reacted loaded RFID complexes is subjected to RFID interrogation. The presence of a particular reacted loaded RFID complex in the solution phase indicates a binding event between that reacted loaded RFID complex and the target agent to which it specifically binds, thereby confirming the presence of the target agent in the sample. Typically, in such an embodiment, different RFID tags are assigned to each target agent to be detected so that an RFID reader can discern which target agents were in the sample by virtue of which of the RFID tags remain in solution. For example, different RFID tags may be encoded with different information to allow their recognition by an RFID reader.

In some embodiments of the invention, a method is provided to determine the presence of a target nucleic acid in a sample comprising mixing the sample with a capture oligo complementary to the target nucleic acid under conditions that promote specific hybridization between the target nucleic acid and the capture oligo to create “hybridized complexes.” The hybridized complexes are exposed to tracking complexes that include a tracking component and a capture moiety that specifically binds to the hybridized complexes. The tracking complexes that bind to the hybridized complexes are separated from those that do not, and the tracking complexes that bound to the hybridized complexes are subjected to interrogation by a detection device. A signal detected from the tracking components is indicative of the presence of a target agent in the sample. In certain embodiments, the signal that indicates the presence of a target agent in the sample is altered relative to a baseline signal. In some embodiments, the tracking component is an RFID tag. In some embodiments, the tracking component is conjugated to a plurality of capture moieties. In certain embodiments, the capture moiety is an antibody. In specific embodiments, chromatography and/or immobilized binding partners are used to separate tracking complexes that bind to the hybridized complexes from those that do not. In certain embodiments, a plurality of different target agents is detected simultaneously.

In some embodiments of the invention, a method is provided to determine the presence or sequence of a target nucleic acid in a sample comprising mixing the sample with a first complex comprising a tracking component and a capture oligo complementary to the target nucleic acid under conditions that promote specific hybridization between the target nucleic acid and the capture oligo to create “hybridized complexes.” The hybridized complexes are exposed to a moiety that specifically binds to the hybridized complexes, e.g., with the duplex created by hybridization of the capture oligo to the target nucleic acid, wherein the moiety is capable of affecting a signal from the tracking component. The presence or sequence of the target nucleic acid is determined by detecting an effect in the signal by the moiety. In more specific embodiments the effect comprises enabling the tracking component to display the signal, or enabling the tracking component to alter the signal relative to a baseline signal generated in the absence of the moiety. Such an alteration may be, for example, an increase, decrease, enhancement, and/or altered frequency or wavelength of the signal. In certain embodiments, the signal that indicates the presence of a target agent in the sample is altered relative to a baseline signal. In some embodiments, the tracking component is an RFID tag. In some embodiments, the tracking component is conjugated to a plurality of capture oligos. In certain embodiments, the moiety comprises an antibody, a nucleic acid-binding protein, an intercalating agent, a metallo complex, a cis-platen, a heme compound, a ruthenium-containing compound, a platinum-containing compound, iron-containing compound, a transition metal-containing compound, or a combination or plurality thereof. In specific embodiments, chromatography and/or immobilized binding partners are used to separate capture oligos (conjugated to tracking components) that bind to the target nucleic acid from those that do not. In certain embodiments, a plurality of different target nucleic acids is detected simultaneously. In certain embodiments, the first complex is immobilized to a matrix. Such methods may also be used to, for example, determine the genotype of a SNP or detect the presence or absence of a genetic mutation.

In certain embodiments, an oligonucleotide may be coupled to an RFID device, e.g., to provide confirmation of a binding event between a loaded RFID complex and a target agent and/or immobilized binding partner. The use of such oligonucleotides (“oligos”) is described in detail in the co-pending applications, U.S. Ser. No. 60/850,016, filed Oct. 6, 2006, entitled “Scaffold-Bound Capture Moieties and Uses Thereof;” and U.S. Ser. No. 11/703,103, filed Feb. 7, 2007, entitled “Device and Methods for Detecting and Quantifying One or More Target Agents,” both of which are hereby incorporated by reference in their entireties for all purposes. Briefly, in some embodiments, an oligo chip is used in a method of electrochemically confirming the presence of a target agent in a sample. Such embodiment may include, in varied orders or combinations, the use of (1) an electrode-associated oligo, (2) a capture-associated oligo that is complementary to the electrode-associated oligo, where the capture-associated oligo is associated with an RFID device, which also comprises a capture moiety specific for the target agent to be detected (“loaded RFID-oligo complex”), (3) immobilized binding partners to the target agent or target agent/capture moiety complex, and (4) a sample suspected of containing the target agent. The method includes mixing the sample suspected of containing the target agent with the loaded RFID-oligo complex to allow the capture moiety to bind the target agent to form reacted loaded RFID-oligo complexes in a mixture. The mixture is contacted with immobilized binding partners to the target agent or target agent/capture moiety complex. The reacted loaded RFID-oligo complexes can react with the immobilized binding partners, thereby removing reacted loaded RFID-oligo complexes from solution to create an immobilized phase. The solution phase containing the unreacted loaded RFID-oligo complexes is separated from the immobilized phase, and the immobilized phase is washed. The immobilized phase is scanned with an RFID reader, and the RFID tags in the reacted loaded RFID-oligo complexes can be identified, e.g., based on identification numbers or other information encoded therein. The presence of an immobilized reacted loaded RFID-oligo complex indicates a presence of the target agent in the sample, and indicates binding events between (a) the target agent and the capture moiety, and (b) the immobilized binding partner and the target agent or target agent/capture moiety complex. The capture-associated oligos associated with the immobilized reacted loaded RFID-oligo complexes are released into solution and contacted with the electrode-associated oligo, where a hybridization event between the electrode-associated oligo and the capture-associated oligo confirms that a target agent was present in the sample. The hybridization event can be detected by electrochemical detection, or any other detection method that can detect a hybridization event. Electrochemical detection can be direct or indirect. In some embodiments, the capture-associated oligos that are associated with the reacted loaded RFID-oligo complexes may be subjected to a cleavage reaction and/or a linear or logarithmic amplification step after the reacted loaded RFID-oligo complexes are separated from the unreacted loaded RFID-oligo complexes but before being contacted with the electrode-associated oligos. These methods are further described, e.g., in the co-pending applications listed supra. Though electrochemical detection is described specifically in this paragraph, it should be noted that detection could be achieved by other techniques known in the art including fluorescence, chemiluminescence, colorimetric assays and the like. In some embodiments, detection of hybridization occurs prior to scanning the reacted loaded RFID-oligo complexes with an RFID reader; in other embodiments detection of hybridization occurs subsequent to scanning the reacted loaded RFID-oligo complexes with an RFID reader; and in still further embodiments, detection of hybridization occurs concurrently with scanning the reacted loaded RFID-oligo complexes with an RFID reader. In further related embodiments, the binding partners are specific for capture moieties that have not bound target agent and, as such, immobilize the unreacted loaded RFID-oligo complexes rather than the reacted loaded RFID-oligo complexes as described above. In such embodiments, the solution phase comprising the reacted loaded RFID-oligo complexes is subjected to RFID interrogation prior to or subsequent to hybridization of the capture-associated oligo with the electrode-associated oligo. The presence of a particular reacted loaded RFID-oligo complex in the solution phase indicates a binding event between that reacted loaded RFID-oligo complex and a particular target agent, and confirms the presence of the target agent in the sample.

In certain embodiments of the invention, one or more target agents are labeled with, for example, tracking components or magnetic labels. In some embodiments of the invention, one or more target agents are immobilized prior to addition of a tracking complex that includes a tracking component and a capture moiety that specifically interacts with the target agent.

In further embodiments of the invention, an in vivo method of determining the presence of a target agent that includes administering a first complex comprising a tracking component capable of generating a signal and a binding moiety capable of associating with the target agent to a patient in a clinically-effective amount, scanning the patient with a reader capable of detecting the signal, and detecting the signal. In certain embodiments, the binding moiety comprises an antibody, an antigen, a protein, a ligand, a nucleic acid, a receptor, a toxin, an immunoglobulin, a metabolite, a hormone, a receptor binding agent, or a combination or plurality thereof. In certain embodiments, the binding moiety is capable of binding a cancer marker, a genetic mutation, a nucleic acid sequence, a protein, a metabolite, a toxin, a drug, a pathogen, a microorganism, a virus, or a combination or plurality thereof. In some embodiments, the tracking component is an RFID device. In some embodiments, the method further includes interrogating the tracking component with a reader capable of generating a response signal from the tracking component of sufficient energy to destroy a cell associated with the target agent. For example, the sufficient energy can be equivalent to 0.25-10 gray (Gy). In some embodiments, the cell is a cancer cell, a microorganism, a pathogen, or a virally-infected cell.

Further provided is a composition comprising a matrix containing at least one immobilized binding partner, a target agent associated with the immobilized binding partner, and a tracking component associated with the target agent. In some embodiments, the composition further comprises a capture moiety, a reactive group, and/or a detection device. Also provided is a radio frequency signal used to determine appropriate medical intervention for a patient whereby the radio frequency signal is indicative of the presence of a target agent in a sample taken from the patient.

In a further embodiment, a composition is provided containing a tracking component, a biomolecule, and a metal-containing compound that is a component of an antenna, which affects a signal between the tracking component and a reader. In some embodiments, the antenna enables detection of the tracking component by the reader. In some embodiments, the metal-containing compound alters one or more characteristics of the signal, such as, for example, signal strength (increase or decrease), frequency, and/or wavelength. The biomolecule can be any biomolecule, for example, a DNA (ssDNA or dsDNA), a protein, or a cyclic organic compound. The metal-containing compound can be associated with the biomolecule via interactions such as intercalation, major/minor groove binding/association, complexation, covalent interaction, or noncovalent interaction. The metal-containing compound can be associated with the biomolecule to form M-DNA. The metal-containing compound can comprise at least one transition metal, electroactive marker, and/or ligand, such as a sigma or pi donor.

Also provided is a system for determining a presence of a target agent in a sample comprising, in various orders and combinations, the sample, tracking components associated with the target agent, a detection device capable of detection the tracking components, and/or immobilized binding partners. Also provided is a diagnostic tool for detecting a target agent in a sample comprising a capture moiety that binds to the target agent to form a target agent-capture moiety complex, a tracking component that can be associated with the target agent-capture moiety complex, and a detection device.

In addition, methods of doing business are provided that comprise the use of a signal related to a presence of a tracking component to determine appropriate medical intervention for a patient. For example, such a method can comprise obtaining a sample from a patient and mixing the sample with tracking complexes that include a tracking component and a capture moiety that binds to a target agent in the sample. Immobilized binding partners facilitate separation of tracking complexes that bound target agents from tracking complexes that did not bind target agents. The tracking complexes that bound the target agents are interrogated by a detection device, and a signal is detected that is indicative of a presence of the target agent in the sample. In certain embodiments, the signal that indicates the presence of a target agent in the sample is altered relative to a baseline signal. The presence of the target agent in the sample provides information useful in determining appropriate medical intervention for the patient.

These and other objects, advantages, and features of the invention will become apparent to those persons skilled in the art upon reading the details of the methods and formulations as more fully described below.

### DESCRIPTION OF THE FIGURES

So that the manner in which the features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments

FIG. 1 33 illustrates an embodiment of an RFID point-of-care device.

FIG. 2 1A&B provides a pictorial representation of an RFID device in accordance with certain embodiments of the present invention. The surface of a device is shown in A, while B shows a cross-section of a device displaying an embedded RFID tag.

FIG. 3 1C provides pictorial representation of several embodiments of RFID devices with coatings on a flat or wafer-like configuration of an RFID tag.

FIG. 4 2 provides a pictorial representation of an RFID device in accordance with another embodiment of the present invention. FIG. 2A shows a side view of the RFID device with a polymer and plurality of capture moieties, FIG. 2B shows one side of the RFID device comprising the RFID tag, and FIG. 2C shows the other side of the RFID device comprising the polymer and capture moieties.

FIG. 5 13 illustrates an embodiment of the present invention where a coated reacted RFID complex has been mixed with binding partners that have been immobilized on microspheres.

FIG. 6 26 illustrates various RFID devices for use in the present invention.

FIG. 7 3 illustrates an embodiment of a method using an RFID for identification of a target agent in a sample.

FIG. 8 4 illustrates an embodiment of a method using an RFID device for identification of a target agent in a sample.

FIG. 9 5 illustrates a microarray comprising a plurality of RFID.

FIG. 10 illustrates a multiplexed embodiment of the present invention using an RFID device for identification of a target agent in a sample.

FIG. 11 illustrates an embodiment of the present invention using an RFID device illustrated for identification of a nucleic acid in a sample.

FIG. 12 illustrates an embodiment of the present invention using an RFID device for identification of a nucleic acid in a sample.

FIG. 13 15A illustrates an embodiment of the present invention using RFID devices for identification of nucleic acids.

FIG. 14 15B illustrates an embodiment of the present invention using RFID devices for identification of nucleic acids

FIG. 15 20 illustrates an embodiment of the present invention using an RFID device for genotyping of a single nucleotide polymorphism.

FIG. 16 18 illustrates an embodiment of the present invention using an RFID device for identification of a nucleic acid in a sample.

FIG. 17 16 illustrates an embodiment of the present invention using an RFID device for identification of a nucleic acid in a sample.

FIG. 18 17 illustrates an embodiment of the present invention using an RFID device for identification of a nucleic acid in a sample.

FIG. 19 (A) illustrates an embodiment of the present invention using RFID devices for identification of nucleic acids.

FIG. 20 (B) illustrates an embodiment of the present invention using RFID devices for identification of nucleic acids.

FIG. 21 (C) illustrates an embodiment of the present invention using RFID devices for identification of nucleic acids

FIG. 22 21 illustrates an embodiment of the present invention using an RFID device for identification of a nucleic acid in a sample.

FIG. 23 illustrates an embodiment of the present invention using an RFID device for identification of a nucleic acid in a sample.

FIG. 24 illustrates an embodiment of the present invention using an RFID device for identification of a nucleic acid in a sample.

FIG. 25 illustrates an embodiment of the present invention using an RFID device for identification of a nucleic acid in a sample.

FIG. 26 27 illustrates an embodiment of a method using an RFID device for identification of a target agent in a sample.

FIG. 27 28 illustrates an embodiment of the present invention using an RFID device for identification of a nucleic acid in a sample.

FIG. 28 29 illustrates an embodiment of the present invention using an RFID device for identification of a nucleic acid in a sample.

FIG. 29 30 illustrates an embodiment of the present invention using an RFID device for genotyping of a single nucleotide polymorphism.

FIG. 30 31 illustrates an embodiment of the present invention using an RFID device for identification of a nucleic acid in a sample.

FIG. 31 32 illustrates an embodiment of the present invention using an RFID device for identification of a nucleic acid in a sample.

FIG. 32 14 illustrates one embodiment of a device for separating RFID devices., where reacted loaded RFID complexes can be separated from unreacted loaded RFID complexes by centrifugation.

FIG. 33 6 is a schematic diagram of a system formed in accordance with one embodiment of the present invention.

FIG. 34 7 is a block diagram of a computer-implemented system architecture formed in accordance with an embodiment of the present invention.

FIG. 35 8 shows a computer-implemented system architecture in accordance with an embodiment of the present invention.

FIG. 36 9 shows a computer-implemented system architecture in accordance with an embodiment of the present invention.

### DETAILED DESCRIPTION OF THE INVENTION

Before the present assays, detection methods and kits are described, it is to be understood that this invention is not limited to the particular devices and detection methods described, and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural referents unless the context clearly dictates otherwise. Thus, for example, reference to “a sample” refers to one or mixtures of samples, and reference to “the assay” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for all purposes, e.g., the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. Also encompassed within the invention is the concept that the upper and lower limits of these smaller ranges may independently be included in the smaller ranges subject to any specifically excluded limit in the stated range. Where the stated range includes one or both of the limits, ranges excluding either of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of cell culture, antibody production, and nucleic acid synthesis techniques and the like are within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Although the present invention is described with a preferred embodiment utilizing RFID technology, it will be clear to one skilled in the art upon reading the present disclosure that other wireless sensors and actuators may be used to create the devices of the present invention and to carry out the methods as described. It is intended that such subject matter be included in the scope of the invention.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The terms “nucleic acid molecules,” “oligonucleotides,” and “oligos” as used herein refer to linear oligomers of natural or modified nucleic acid monomers or linkages (including, e.g., deoxyribonucleotides, ribonucleotides, and anomeric forms thereof; peptide nucleic acids (PNAs); locked nucleotide acids (LNA); and the like and/or combinations thereof), capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200. Suitable nucleic acid molecules may be prepared by the phosphoramidite method described by Beaucage and Carruthers (Tetrahedron Lett., 22, 1859-1862, 1981), or by the triester method according to Matteucci, et al. (J. Am. Chem. Soc., 103, 3185, 1981), both incorporated herein by reference, or by other chemical methods such as using a commercial automated oligonucleotide synthesizer. Typically, oligonucleotides are single-stranded, but double-stranded or partially double-stranded oligos may also be used in certain embodiments of the invention. Oligonucleotides may also comprise a tag (e.g., detectable label, magnetic bead, etc.) or other component to facilitate detection in or purification or separation from a mixture.

The terms “complementary” and “complementarity” refer to nucleic acid molecules related by base-pairing rules. Complementary nucleotides are, generally, A and T (or A and U), or C and G. For example, for the sequence “5′-AGT-3′,” the perfectly complementary sequence is “3′-TCA-5′.” Methods for calculating the level of complementarity between two nucleic acids are widely known to those of ordinary skill in the art. For example, complementarity may be computed using online resources, such as, e.g., the NCBI BLAST website (ncbi.nlm.nih.gov/blast/producttable.shtml) and the Oligonucleotides Properties Calculator on the Northwestern University website (basic.northwestern.edu/biotools/oligocalc.html). Two single-stranded RNA or DNA molecules may be considered substantially complementary when the nucleotides of one strand, optimally aligned and with appropriate nucleotide insertions or deletions, pair with at least about 80% of the nucleotides of the other strand, usually at least about 90% to 95%, and more preferably from about 98 to 100%. Two single-stranded nucleic acid molecules are considered perfectly complementary to one another when the nucleotides of one strand, optimally aligned and with appropriate nucleotide insertions or deletions, pair with 100% of the nucleotides of the other strand. Alternatively, substantial complementarity exists when an first oligonucleotide will hybridize under selective hybridization conditions to a second oligonucleotide. Selective hybridization conditions include, but are not limited to, stringent hybridization conditions. Selective hybridization occurs in one embodiment at least about 65% of the nucleic acid monomers within a first oligonucleotide over a stretch of at least 14 to 25 monomers pair with a perfectly complementary monomer within a second oligonucleotide, preferably at least about 75%, more preferably at least about 90%. See, M. Kanehisa, Nucleic Acids Res. 12, 203 (1984), incorporated herein by reference. For shorter nucleotide sequences selective hybridization occurs when at least about 65% of the nucleic acid monomers within a first oligonucleotide over a stretch of at least 8 to 12 nucleotides pair with a perfectly complementary monomer within a second oligonucleotide, preferably at least about 75%, more preferably at least about 90%. Stringent hybridization conditions will typically include salt concentrations of less than about 1 M, more usually less than about 500 mM and preferably less than about 200 mM. Hybridization temperatures can be as low as 5° C. and are preferably lower than about 30° C. However, longer fragments may require higher hybridization temperatures for specific hybridization. Hybridization temperatures are generally at least about 2° C. to 6° C. lower than melting temperatures (Tₘ), which is defined below.

The term “melting temperature” or Tₘ is commonly defined as the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+16.6(log₁₀[Na⁺])0.41(%[G+C])−675/n−1.0m, when a nucleic acid is in aqueous solution having cation concentrations of 0.5 M, or less, the (G+C) content is between 30% and 70%, n is the number of bases, and m is the percentage of base pair mismatches (see e.g., Sambrook J et al., “Molecular Cloning, A Laboratory Manual, 3^{rd} Edition, Cold Spring Harbor Laboratory Press (2001)). Other references include more sophisticated computations, which take structural as well as sequence characteristics into account for the calculation of Tₘ.

A “capture moiety” refers to a molecule or a portion thereof that can be used to preferentially bind and separate a molecule of interest (a “target agent”) from a sample. The term “capture moiety” as used herein refers to any molecule, natural, synthetic, or recombinantly-produced, or portion thereof, with the ability to bind to or otherwise associate with a target agent in a manner that facilitates detection of the target agent in accordance with the methods of the present invention. For example, in certain embodiments the binding affinity of the capture moiety must be sufficient to allow collection, concentration, and/or separation of the target agent from a sample. Suitable capture moieties include, but are not limited to, immunoglobulins, antibodies, antigen-binding regions of antibodies, antigens, epitopes, cell receptors (e.g., cell surface receptors), enzymes, enzyme substrates, enzyme inhibitors, peptidomimetic molecules, and cell receptor ligands, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Similarly, capture moieties may also include but are not limited to toxins, venoms, receptors (e.g., intracellular receptors that mediate the effects of various small ligands, including steroids, hormones, retinoids and vitamin D, peptides) and ligands thereof, drugs (e.g., opiates, steroids, etc.), lectins, metabolites, sugars, oligosaccharides, other proteins, phospholipids, and structured nucleic acids such as aptamers and the like. In particular embodiments, a capture moiety is a nucleic acid (e.g., a “capture oligo”) that is complementary to a nucleic acid target agent, e.g., viral DNA or RNA. Those of skill in the art will readily appreciate that molecular interactions other than those listed above are well described in the literature and may also serve as capture moiety/target agent interactions. In certain embodiments, capture moieties are associated with scaffolds, and in other embodiments capture moieties are conjugated to capture-associated oligos.

A “loaded RFID complex” refers to a radio frequency identification (RFID) device or other wireless sensor or actuator that has one or more capture moieties affixed to or otherwise associated with it. The term “reacted loaded RFID complex” is used in reference to loaded RFID complexes comprising at least one capture moiety bound (directly or indirectly) to a target agent from a sample. The term “unreacted loaded RFID complex” is used in reference to loaded RFID complexes comprising no capture moieties bound (directly or indirectly) to a target agent from a sample. In certain embodiments, multiple capture moieties can be associated with an RFID device, such that a positive signal may indicate the presence of one or more of multiple target agents (e.g., one or more types of hepatitis, HPV, or the like). These embodiments are not intended to indicate the presence of a specific type of the target class (e.g., HPV 16 or Hepatitis A), but instead give a positive indication of the presence of the general class of target agent.

The term “loaded RFID-oligo complex” refers to a loaded RFID that has one or more capture-associated oligonucleotides affixed to or otherwise associated with it. The term “reacted loaded RFID-oligo complex” is used in reference to loaded RFID-oligo complexes comprising at least one capture moiety bound (directly or indirectly) to a target agent from a sample. The term “unreacted loaded RFID-oligo complex” is used in reference to loaded RFID-oligo complexes comprising no capture moiety bound (directly or indirectly) to a target agent from a sample.

The term “binding partner” as used herein refers to any molecule, natural, synthetic, or recombinantly-produced, with the ability to bind to a target agent and/or capture moiety in the methods of the present invention. For example, in some embodiments a binding partner is a molecule or portion thereof that preferentially binds to a moiety of the target agent different from a moiety of the target agent that is bound by a capture moiety, such that both the capture moiety and the binding partner may be simultaneously bound to the target agent. In other embodiments, a “binding partner” may preferentially bind to a capture moiety/target agent complex. Alternatively, in certain embodiments immobilized binding partners will bind unreacted capture moieties (i.e., those that have not bound to target agent). The binding affinity of the binding partner must be sufficient to allow collection of the target agent and/or capture moiety from a sample and/or sample mixture. Suitable binding partners include, but are not limited to, antibodies, antigen-binding regions of antibodies, antigens, epitopes, cell receptors (e.g., cell surface receptors), enzymes, enzyme substrates, enzyme inhibitors, peptidomimetic molecules, and cell receptor ligands, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). As with capture moieties, binding partners may also include but are not limited to toxins, venoms, intracellular receptors (e.g., receptors that mediate the effects of various small ligands, including steroids, hormones, retinoids and vitamin D, peptides), drugs (e.g., opiates, steroids, etc.), lectins, sugars, oligosaccharides, other proteins, and phospholipids. In particular embodiments, the binding partner may be a nucleic acid that is complementary to a nucleic acid target agent, e.g., viral DNA or RNA. Those of skill in the art will readily appreciate that molecular interactions other than those listed above are well described in the literature and may also serve as the basis for binding parter interactions with target agents and/or capture moieties. The binding partners can be affixed/immobilized directly or indirectly to a matrix such as a vessel wall, to particles or beads (as described in more detail infra), or to other suitable surfaces to form “immobilized binding partners.” Those of skill in the art will readily understand the versatility of the nature of this immobilized binding partner. Essentially, any ligand and receptor can be utilized to serve as capture moieties, target agents and binding partners, as long as the target agent is appropriate for detection for the pathology or condition interrogated. Suitable ligands and receptors include an antibody or fragment thereof to be recognized by a corresponding antigen or epitope, a hormone to be recognized by its receptor, an inhibitor to be recognized by its enzyme, a co-factor portion to be recognized by a co-factor enzyme binding site, a binding ligand to be recognized by its substrate, and the like.

By “preferentially binds” and it is meant that a specific binding event between a first and second molecule occurs at least 20 times or more, preferably 50 times or more, more preferably 100 times or more, and even 1000 times or more often than a nonspecific binding event between the first molecule and a molecule that is not the second molecule. For example, a capture moiety can be designed to preferentially bind to a given target agent at least 20 times or more, preferably 50 times or more, more preferably 100 times or more, and even 1000 times or more often than to other molecules in a biological solution. Also, an immobilized binding partner, in certain embodiments, will preferentially bind to a target agent, capture moiety, or capture moiety/target agent complex. Binding will be recognized as existing when the Kₐ is at 10⁷ l/mole or greater, preferably 10⁸ l/mole or greater. In the embodiment where the capture moiety or binding partner comprises antibody, the binding affinity of 10⁷ l/mole or more may be due to (1) a single monoclonal antibody (e.g., large numbers of one kind of antibody) or (2) a plurality of different monoclonal antibodies (e.g., large numbers of each of a plurality of different monoclonal antibodies) or (3) large numbers of polyclonal antibodies. It is also possible to use combinations of (1)-(3). For example, a four-fold differential in binding affinity may be accomplished by using several different antibodies as per (1)-(3) above and as such some of the antibodies in a mixture could have less than a four-fold difference. In certain embodiments, an indication that no binding occurs means that the equilibrium or affinity constant Kₐ is 10⁶ l/mole or less. Antibodies may be designed to maximize binding to the intended antigen by designing the peptides to specific epitopes that are more accessible to binding, as can be predicted by one skilled in the art.

A “target agent” is a molecule of interest in a sample that is to be detected through the methods of the instant invention. For example, in certain embodiments the target agent is captured through preferential binding with a capture moiety. In one such embodiment, the capture moiety is an antibody and the target agent is any molecule containing the epitope against which the antibody is generated, or an epitope specifically bound by the antibody. In another embodiment, the capture moiety is a protein specifically bound by an antibody, and the antibody itself is the target agent. Target agents also may include but are not limited to organic and inorganic molecules (e.g., biomolecules), receptors (e.g., cell surface receptors) and ligands thereof, intracellular receptors (e.g., receptors which mediate the effects of various small ligands, including steroids, hormones, retinoids and vitamin D, peptides) and ligands thereof, metabolites, steroids, hormones, lectins, sugars, oligosaccharides, proteins, enzymes, agonists, antagonists, antibodies, antigens, phospholipids, toxins, venoms, drugs (e.g., opiates, steroids, etc.), small molecules, nucleic acids (e.g., DNA, RNA, PNA, combinations thereof, etc.), therapeutic molecules (including therapeutic and abused drugs, antibiotics, etc.), naturally occurring molecules with known physiological function (including hormones, cytokines, proteins, lipids, carbohydrates, cellular membrane antigens and receptors (neural, hormonal, nutrient, and cell surface receptors) or their ligands, etc.), whole cells (including prokaryotic (such as pathogenic bacteria) and eukaryotic cells, including mammalian tumor cells), viruses (including retroviruses, herpesviruses, adenoviruses, lentiviruses, etc.), spores, or any portion, fragment, epitope, or combination thereof. Essentially, any ligand or receptor can be utilized to serve as capture moieties, target agents or binding partners, as long as the target agent is appropriate for detection of the pathology or condition of interest (e.g., a pathology or condition that may be diagnosed by detection or quantitation of the target agent in a sample) interrogated. Suitable ligands and receptors include an antibody or fragment thereof to be recognized by a corresponding antigen or epitope, a hormone to be recognized by its receptor, an inhibitor to be recognized by its enzyme, a co-factor portion to be recognized by a co-factor enzyme binding site, a binding ligand to be recognized by its substrate, and the like. Those of skill in the art readily will appreciate that molecular interactions other than those listed above are well described in the literature and may also serve as capture moiety/target agent or binding partner/target agent interactions.

The term “sample” in the present specification and claims is used in its broadest sense and can be, by non-limiting example, any sample that is suspected of containing the target agents to be detected. It is meant to include specimens or cultures (e.g., microbiological cultures), and biological and environmental specimens as well as non-biological specimens. Biological samples may comprise animal-derived materials, including fluid (e.g., blood (whole, serum, etc.), saliva, urine, semen, lymph, amniotic fluid, peritoneal fluid, pleural fluid, pericardial fluid, ascetic fluid, spinal fluid, synovial fluid, etc.), solid (e.g., feces, blood cells, etc.), or tissue (e.g., buccal, organ-specific, skin, fine needle biopsy samples, etc.) and tissue homogenates, as well as liquid and solid food and feed products and ingredients such as dairy items, vegetables, meat and meat by-products, and waste. Samples may also include sections of tissues such as frozen sections taken for histological purposes. Biological samples may be obtained from, e.g., humans, any domestic or wild animals, plants, bacteria or other microorganisms, etc. Environmental samples can include environmental material such as surface matter, soil, water (e.g., contaminated water), air and industrial samples, as well as samples obtained from food and dairy processing instruments, apparatus, equipment, utensils, disposable and non-disposable items. These examples are not to be construed as limiting the sample types applicable to the present invention. Those of skill in the art would appreciate and understand the particular type of sample required for the detection of particular target agents and an appropriate procedure for sample preparation (see, e.g., Pawliszyn, J., Sampling and Sample Preparation for Field and Laboratory, (2002); Venkatesh Iyengar, G., et al., Element Analysis of Biological Samples: Principles and Practices (1998); Drielak, S., Hot Zone Forensics: Chemical, Biological, and Radiological Evidence Collection (2004); and Nielsen, D. M., Practical Handbook of Environmental Site Characterization and Ground-Water Monitoring (2005)).

The term “antibody” as used herein refers to an entire immunoglobulin or antibody or any fragment of an immunoglobulin molecule that is capable of specific binding to a target agent of interest (an antigen). Examples of such antibodies include complete antibody molecules, antibody fragments, such as Fab, F(ab′)₂, CDRS, V_{L}, V_{H}, and any other portion of an antibody that is capable of specifically binding to an antigen. An IgG antibody molecule is composed of two light chains linked by disulfide bonds to two heavy chains. The two heavy chains are, in turn, linked to one another by disulfide bonds in an area known as the hinge region of the antibody. A single IgG molecule typically has a molecular weight of approximately 150-160 kD and contains two antigen binding sites. An F(ab′)₂ fragment lacks the C-terminal portion of the heavy chain constant region, and has a molecular weight of approximately 110 kD. It retains the two antigen binding sites and the interchain disulfide bonds in the hinge region, but it does not have the effector functions of an intact IgG molecule. An F(ab′)₂ fragment may be obtained from an IgG molecule by proteolytic digestion with pepsin at pH 3.0-3.5 using standard methods such as those described in Harlow and Lane, supra. Preferred antibodies for assays of the invention are immunoreactive or immunospecific for, and therefore specifically and preferentially bind to, a given antigen (e.g., a protein, small molecule, nucleic acid, etc.) that is indicative of a pathology or condition of interest, and are not limited to the G class of immunoglobulin used in the above cited example. A “purified antibody” refers to that which is sufficiently free of other proteins, carbohydrates, and lipids.

The term “capture reaction” is commonly used in reference to the mixing/contacting of an RFID device comprising to a capture moiety and a sample under conditions that allow the capture moiety to attach to, bind or otherwise associate with target agent in the sample. For example, a “capture reaction” can involve mixing/contacting of one or more loaded RFID complexes and a sample under conditions that allow a capture moiety of the loaded RFID complexes to attach to, bind or otherwise associate with a target agent in the sample.

The term “matrix” means any surface.

The term “RFID reader” refers to a device used to interrogate an RFID of an RFID device.

The term “reactive group” refers to a moiety or molecule that is designed such that a binding partner, an immobilized binding partner, target agent, capture moiety, and/or the like will preferentially bind to it. Suitable reactive groups include, but are not limited to, antibodies, antigen-binding regions of antibodies, antigens, epitopes, cell receptors, enzymes, enzyme substrates, enzyme inhibitors, peptidomimetic molecules, and cell receptor ligands, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Reactive groups may also include but are not limited to toxins, venoms, intracellular receptors (e.g., receptors that mediate the effects of various small ligands, including steroids, hormones, retinoids and vitamin D, peptides), drugs (e.g., opiates, steroids, etc.), lectins, sugars, oligosaccharides, other proteins, and phospholipids. In particular embodiments, the reactive group may be a nucleic acid that is complementary to an immobilized nucleic acid, e.g., viral DNA or RNA. Those of skill in the art will readily appreciate that a number of reactive groups based upon other molecular interactions than those listed above are well described in the literature and may also serve as reactive groups.

It should be understood by those skilled in the art that terms such as “target”, “agent”, “moiety”, “antigen”, “antibody”, “molecule” and the like should be interpreted in the context in which they appear, and should be given the broadest interpretation possible unless specifically indicated.

### General

The present invention relates to devices and methods for the detection of one or more target agents in a sample as well as for the detection of binding events between two or more molecules. The present invention provides devices comprising a tracking component for use in the identification of a target agent in a sample, preferably a biological or environmental sample. Such a tracking component may be associated (directly or indirectly) with one or more capture moieties that preferentially bind to one or more target agents of interest. The presence of a tracking component can be identified using an authorization/interrogation device with the ability to identify the particular tracking component(s) associated with a target agent/capture moiety complex. In some embodiments, a tracking component is an RFID tag and a device comprising an RFID tag is an RFID device. Further, based on the instant disclosure it will be clear to one of skill in the art that methods disclosed herein may be adapted to detect a plurality of target agents although many examples presented herein describe detection of a single target agent.

RFID devices have utility in detecting specific interaction between biomolecules in individual assays. For example, a first biomolecule may be conjugated to an RFID tag and a second biomolecule may be attached or otherwise immobilized onto a matrix. The first biomolecule-RFID complex is exposed to the immobilized second biomolecule under conditions that promote interaction between the two biomolecules. A binding event between the first and second biomolecules causes immobilization of the RFID tag. RFID interrogation of the immobilized phase of the mixture indicates if there are immobilized RFID tags, and therefore if there was binding between the first and second biomolecules.

RFID devices also have utility in detecting the presence of a target agent in a sample. For example, an RFID tag is conjugated to a capture moiety specific for the target agent to form a loaded RFID complex, which is exposed to the sample under conditions that promote binding between the capture moiety and any target agent present in the sample to form a reacted loaded RFID complex. The resulting mixture is exposed to immobilized binding partners specific for the target agent/capture moiety complex, thereby immobilizing the reacted loaded RFID complex. Any unreacted loaded RFID complex (i.e., not bound to target agent) remains in solution and is subsequently removed. An RFID tag in the immobilized phase is subjected to RFID interrogation, which identifies the RFID tag of any immobilized reacted loaded RFID complex, thereby indicating the presence of the target agent in the sample.

In addition, RFID devices also have utility in confirming the identification of a target agent in a sample that is a identified through other means, e.g., electrochemical detection, fluorescent detection, and the like, thus minimizing the chances of a false positive, and in remote testing for a particular agent. In addition to their role in identification of a target agent, RFID diagnostic devices can provide additional information (e.g., data on the use, manufacture, and conditions used for the performed capture reactions) for the purpose of accurately collecting and/or monitoring diagnostic testing.

The RFID devices of the invention can be produced in a disposable format, intended to be used for a single detection experiment or a series of detection experiments and then thrown away. The RFID devices of the invention can be produced in a portable format allowing for field use where other instrumentation or the like may be inaccessible or otherwise impractical.

The present invention is useful for utilizing both stand-alone RFID tracking technology and networked RFID tracking technology. For example, stand-alone uses include confirmation of the identity of a particular target agent when used in conjunction with a complementary target agent assay system. It can also provide important identity and location information for the RFID devices, e.g., confirming the date of manufacture, batch number, etc. for quality control purposes. Networked RFID systems can be useful for applications such as linking tracking and identity information from a tag to other information stored on networked databases, including the identity of the target agent detected in a sample of interest.

One application of RFID technology is remote healthcare diagnosis and monitoring. The tracking of large numbers of data points can be obtained through the use of an integrated RFID system, with the results of multiple diagnostic capture reactions fed into a global RFID system via multiple local networks. This can benefit the healthcare system as a result of faster diagnosis and treatment of diseases. For example, tracking the presence of infectious diseases, such as flu (e.g., bird flu) and SARS, can be achieved by using RFID diagnostic devices or arrays in multiple countries and feeding the data into an integrated global system designed to capture the data and track movement of the disease. In cases where the diagnosis is particularly private or sensitive, e.g., involves a potentially serious infectious disease or an organism used for bioterrorism, remote diagnosis can afford the ability to quarantine the patient and sample while still providing fast and accurate results via database analysis.

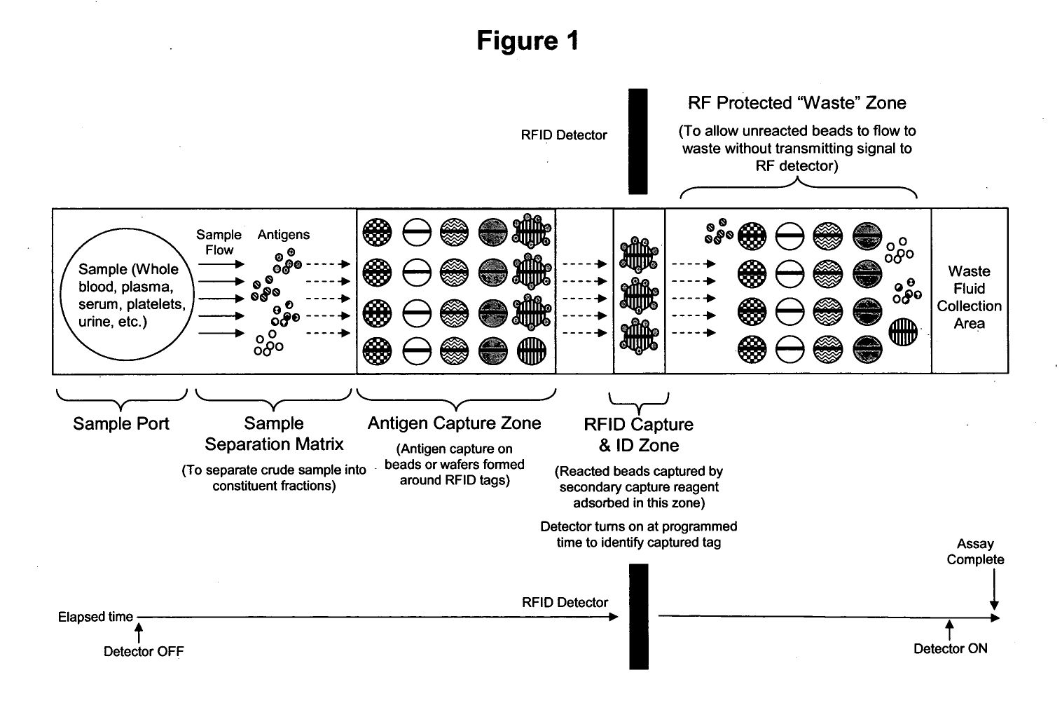

For example, FIG. 1 depicts one embodiment of an RFID point-of-care device. The device comprises a sample port into which a sample is loaded. Such a sample may comprise, e.g., whole blood, plasma, serum, platelets, urine, saliva, semen, lymph, etc. The sample flows into a sample separation matrix in which the crude sample is separated into its constituent fractions comprising target agents and non-target agents. For example, in FIG. 1, the constituent fractions shown are different antigens from the sample, some of which are target antigens and some of which are non-target agents. In an antigen capture zone, target agents (e.g., target antigens) from the sample are captured on RFID complexes (e.g., substrates, such as beads or wafers, comprising RFID tags) to form reacted RFID complexes (i.e., bound to target agent). The sample is passed into an RFID capture and identification zone, in which the reacted RFID complexes are immobilized (e.g., by binding to immobilized binding partners, such as secondary antibodies) and subjected to RFID interrogation by an RFID detector. The unreacted RFID complexes (i.e., not bound to target agent) and the non-target agents pass through the RFID capture and identification zone into the RF protected “waste” zone, which may comprise shielding to prevent RFID tags therein from being detected by the RFID detector, and are collected in a waste fluid collection area. The detection of a particular RFID tag by the RFID detector is indicative that the capture moiety conjugated to that RFID tag has bound the target agent, and therefore that the target agent is present in the sample.

Information relating to the presence or absence of a specific target agent can be transmitted immediately off-site using this technology for verification of the identity of the target agent. In some embodiments, association (direct or indirect) with a target agent enables an RFID tag to transmit a signal to be detected by an RFID reader. In certain related embodiments, the RFID tag may transmit a different signal to be detected by the RFID reader in the absence of target agent. The RFID can be associated with a random identification number, which can then be decoded at a single data collection site. This may be desirable in an instance where information concerning the presence of a target agent is extremely sensitive in nature, e.g., military uses including detection of an agent associated with bioterrorism, or when the results are from a double-blinded testing regime, e.g., clinical trials.

Throughout this application, specific reference will be made to RFID tags (e.g., in certain embodiments), however, it is intended and should be understood that the specific “tracking means” or “tracking component” encompasses other known means known and readily understood by persons having ordinary skill in the art. In some embodiments, the “RFID tag” may be anything that is capable of reflecting radio frequency energy, such as, for example, double-stranded nucleic acid that has been associated with one or more metallo-compounds.

In certain embodiments, an RFID device of the invention comprises an RFID tag associated with a capture moiety that preferentially binds to one or more target agents of interest. The RFID tag may alternatively be bound to or embedded within a matrix onto which the capture moieties are conjugated, or, in some embodiments, the RFID tag itself acts as the matrix for conjugation of the capture moiety. In such instances, the capture moiety can be conjugated (directly or indirectly) to the matrix via a polymer and/or adaptor system, as described more fully herein.

In certain embodiments, a device (e.g., 150×150×8.5 μm) is coated with or encapsulated within a coating that allows attachment of capture moieties to its surface. The device may be coated using a variety of methods, with the coating thickness ranging from a molecular monolayer to encapsulation in a spherical droplet of polymer or hydrogel. In certain embodiments, an encapsulating sphere has a diameter just over 200 μm.

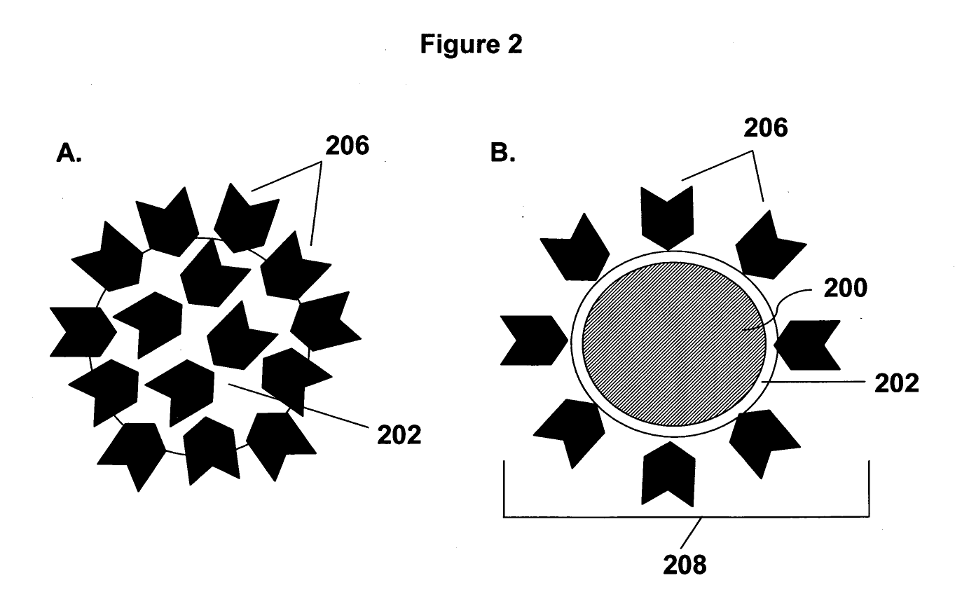

In one specific embodiment provided in FIG. 2, a device of the invention (208) comprises a tracking component (200) (e.g., an RFID tag) coated by a polymer (202), upon which one or more capture moieties for the detection of a target agent are conjugated (206). A depiction of the device as seen from the outside of the device is shown in A, and a cross-section with the tracking component (200) surrounded by a polymer coating (202) is illustrated in B. As seen, in this embodiment, the tracking component itself (200) can serve as the matrix for polymer deposition. In alternative embodiments the tracking component is planar and embedded within spherical (e.g., bead) structure. The capture moiety (206) is dispersed on the polymer surface, and in some embodiments in a uniform fashion.

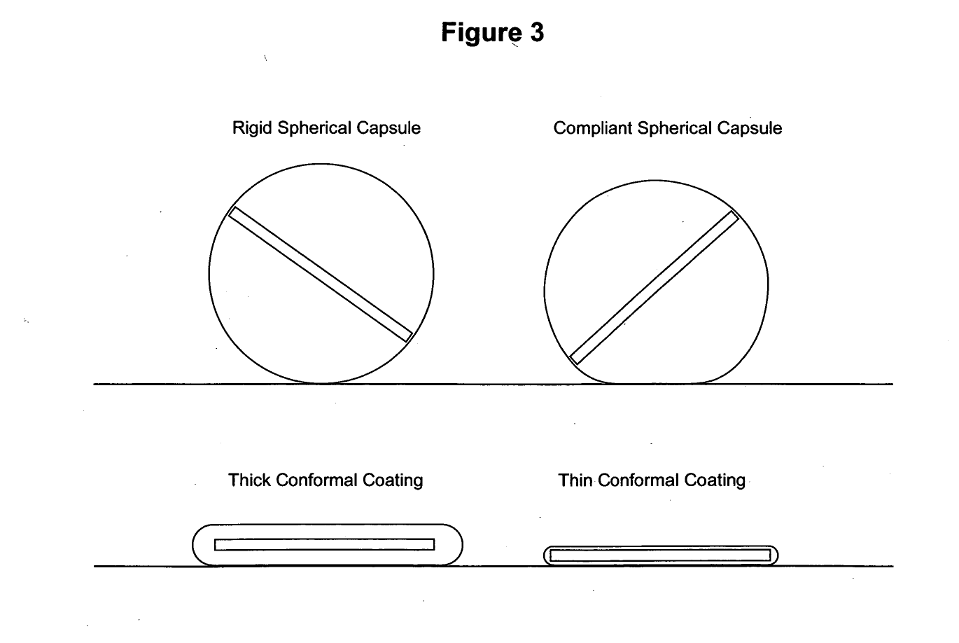

FIG. 3 shows four embodiments—two spherical configurations and two conformal configurations—of coatings used on a wafer-like flat tracking component. Such coatings may have different characteristics depending on the needs of the practitioner of the methods disclosed herein. For example, coatings can be rigid or compliant, and may be thick or thin. In certain instances, a conformal configuration may be preferable, e.g., in embodiments where a capture reaction with immobilized binding partners is performed on a flat surface, as a “face on” binding geometry may reduce hydrodynamic forces and a larger number of bonds may be made between the surface comprising the immobilized binding partners and the RFID device. The use of a pliable material (e.g., as shown for the compliant spherical capsule in FIG. 3) may increase the number of bonds between the surface comprising the immobilized binding partners and the RFID device regardless of the shape of the capsule/coating.

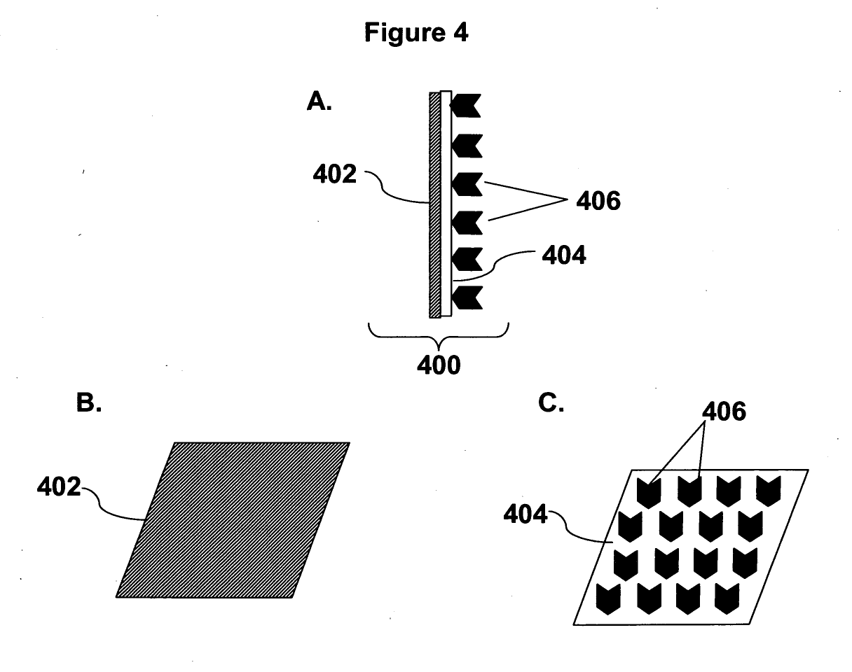

In a specific embodiment shown in FIG. 4, the device (400) provides a planar tracking component (402) with a polymer coating (404) on one surface of the tracking component and one or more capture moieties (406) corresponding to a particular target agent conjugated to the polymer. A side view of the device showing the layers of the device is shown in A. Two planar surfaces of the device are illustrated in B and C: the exposed tracking component (B) and the polymer surface with the conjugated capture moieties (C). As with the embodiments of FIGS. 2 and 3, the tracking component itself (402) can serve as the matrix for polymer deposition. The capture moieties (406) are dispersed on the polymer surface, and in some embodiments in a uniform fashion.

The one or more polymers used in the devices (e.g., RFID devices) of the invention are selected based upon the desired properties to maximize efficiency of binding a capture moiety as well as efficiency of the detection reaction (the interrogation of the tracking component). In embodiments where an adaptor molecule is utilized in conjunction with the polymer, the polymer must be capable of binding to the adaptor molecule, directly or indirectly (e.g., through the use of a linker or peptidic spacer molecule). The thickness of the polymer applied to the tracking component (e.g., RFID tag) is selected to provide an appropriate distance between the surface of the tracking component and the capture moieties used to identify and quantify a target agent in a sample. The polymers used are preferably hydrophilic, e.g., polyacrylamide and polyvinylpyrrolidone being examples of such polymers. If capture-associated oligos are also affixed to or otherwise associated with the device, the polymer chosen will be compatible with the oligo as well. The polymeric material preferably is non-biodegradable and/or biocompatible. In specific embodiments, the polymer is a biocompatible material such as are polylactic acid, polyglycolic acid, polyvinyl alcohol, or similar materials. In some embodiments, the polymer comprises a combination of materials, such as those listed herein.

Polymers for use in the present invention include, but are not limited to, acrylics, vinyls, nylons, polyurethanes, polycarbonates, polyamides, polysulfones, poly(ethylene terephthalate), polylactic acid, polyglycolic acid, polydimethylsiloxanes, polyetheretherketone and polytetrafluoroethylene. In certain embodiments, the device comprises a polymer substrate of polyester, polyolefin or polyurethane. In a further embodiment the device comprises a polymer substrate selected from the group consisting of polyethylene terephthalate, polyethylene, polyether urethane and polysiloxane urethane.

In other specific embodiments, the device comprises an acrylic such as those polymerized from hydroxyethyl acrylate, hydroxyethyl methacrylate, glyceryl acrylate, glyceryl methacrylate, acrylic acid, methacrylic acid, acrylamide and methacrylamide; vinyls such as polyvinyl pyrrolidone and polyvinyl alcohol; nylons such as polycaprolactam; derivatives of polylauryl lactam, polyhexamethylene adipamide and polyhexamethylene dodecanediamide, and polyurethanes; polyethers such as polyethylene oxide, polypropylene oxide, and polybutylene oxide; and biodegradable polymers such as polylactic acid, polyglycolic acid, polydioxanone, polyanhydrides, and polyorthoesters.

If the polymers are deposited on the conductive surface of the device, such deposition may be in any thickness that will allow efficient and accurate detection of a signal, e.g., an RFID signal. Other elements that may need to be taken into consideration in selecting the nature and thickness of the polymer include the sample in which the target agent is to be detected, the size and availability of the capture moiety on the device surface, the capture reaction environment (pH, temperature, etc.). As a general rule, the length of the polymer will directly impact on the efficiency of detection, e.g., RFID detection, so it is preferable to have a thin, uniform film of the polymer that will not impede identification of a target agent and yet will appropriately protect the surface of the device (e.g., RFID chip) and/or provide the best efficacy for detection of low levels of target agent in a sample. The appropriate choice will be well within the skill found in art based on the present disclosure.

In a particular embodiment, the polymer used is Parylene™ (Comelec SA, Switzerland), which has the ability to solidify directly from the gaseous phase at ambient temperature. The treatment results in a linear, crystalline structure that has superior protection qualities at low application thickness. The ability to solidify at ambient temperatures affords the Parylene™ coating high conformity and uniformity, as well as ensuring that they free of porosity or defects.

RFID tags that may be used in the devices of the present invention are preferably small, so as to reduce the amount of polymer and capture moieties needed per device, reduce reaction volumes allowing for decreased cost. However, the methods and devices of the present invention are in no sense limited to the sizes of currently available RFID tags. For example, Hitachi, Ltd. offers both a 0.15×0.15 millimeter (mm), 7.5 micrometer (μm) thick device and a 0.4×0.4 mm (“μ-Chip™”) device. In developing such small devices, Hitachi reduced the distance between each circuit element by using silicon-on-insulator technology, which has an insulating layer in the substrate, rather than a silicon only substrate. Compared to the 0.3×0.3 mm, 60 μm thick IC chip offered by Hitachi, surface area is reduced to a quarter of the original size. Developments in thin chip fabrication technology have also enabled the chip to be reduced to one-eighth the thickness of the 0.3 mm IC chip at the same time.

The μ-Chip™ uses an external antenna to receive radio waves (2.45 GHz microwaves), and transforms it to energy to wirelessly transmit a 128 bit (1038) unique ID number. As the data is written during the fabrication process using ROM (Read-Only-Memory), it is impossible to rewrite the data and thus provides a high level of authenticity. As with the 0.3 mm IC chip, the smaller chip has a double-surface electrode, and therefore despite its even smaller size, connection with the external antenna can be easily achieved, and high productivity maintained.

In one embodiment of the invention, a film of polymer is deposited directly onto the surface of a device (e.g., an RFID device) by electrochemical synthesis from a monomer solution. Electrodeposition of the polymer film can be carried out, e.g., according to the methods disclosed in U.S. Pat. No. 6,770,190 to Milanovski, et al. In such an exemplary method, a solution containing monomers, a polar solvent and a background electrolyte are used for deposition of the polymer. Alternatively, polymers may be deposited by vapor deposition or liquid coating, or a polymer network may be grown from the device surface (e.g., “living” monomer groups can be attached to the surface of the RFID device). In yet another embodiment, the device may be coated with self-assembled monolayers (SAMs) that are attached to a native or grown oxide, or the device may be coated with gold and a thiol-derived SAM is used. Alternatively, the can be suspended in a polymeric or other suitable material, and then encapsulated complexes (e.g., RFID complexes) formed by droplet, spotting, printing or other like method.

Adaptor molecules for conjugation of the capture moiety to the polymer surface may either be immobilized in the polymer film at the electrochemical synthesis stage by adding adaptor molecules to the electrochemical polymerization solution or may be adsorbed onto the surface of the polymer film after electrochemical polymerization. In the former case, a solution of adaptor molecules may be added to the electrodeposition solution immediately before the deposition process. The deposition process works optimally if the storage time of the finished solution does not exceed 30 minutes. Depending on the particular type of test, the concentration of adaptor molecules in the solution may be varied in the range 5-100 μg/mL. On completion of electrodeposition process, the surface may be rinsed successively with deionized water and 0.01 M phosphate-saline buffer solution and, depending on the type of test, may then be placed in a special storage buffer solution containing microbial growth inhibitors or bactericidal agents (e.g., gentamicin), or dried in dust-free air at room temperature.

Where the adaptor molecules are to be adsorbed after completion of the electrodeposition process the following protocol may be used (although it is hereby stated that the invention is in no way limited to the use of this particular method), the device is first rinsed with deionized water and placed in freshly prepared 0.02 M carbonate buffer solution, where it is held for 15-60 minutes. The device is then placed in contact with freshly-prepared 0.02 M carbonate buffer solution containing adaptor molecules at a concentration of 1.00-50.00 μg/mL, by immersing the device in a vessel filled with solution, or by placing a drop of the solution onto the surface of the matrix. The device is incubated with the solution of adaptor molecules, typically for 1-24 hours at +4° C. After incubation, the device is rinsed with deionized water and placed for 1-4 hours in a 0.1 M phosphate-saline buffer solution. Depending on the type of test, the device may then be placed either in a special storage buffer solution containing microbial growth inhibitors or bactericidal agents, or dried in dust-free air at room temperature.

Molecules that may be uses as adaptor molecules are widely known and available to those of ordinary skill in the art. Adaptor molecules need only be capable of conjugating the capture moiety to the polymer surface. In certain embodiments, adaptor molecules are proteins, for example, antibodies. Antibody adaptor molecules can be especially useful when the capture moiety is an antigen. In other embodiments, such as when the capture moiety is an antibody, adaptor molecules may be antigens.

The proteins avidin and streptavidin are preferred for use as adaptor molecules. Avidin consists of four identical peptide sub-units, each of which has one site capable of bonding with a molecule of the co-factor biotin. Biotin (vitamin H) is an enzyme co-factor present in very minute amounts in every living cell and is found mainly bound to proteins or polypeptides. The ability of biotin molecules to enter into a binding reaction with molecules of avidin or streptavidin (a form of avidin isolated from certain bacterial cultures, for example Streptomyces aviation) and to form virtually non-dissociating “biotin-avidin” complexes during this reaction (with a dissociation constant of about 10⁻¹⁵ Mol/l).

When the adaptor molecules are avidin or streptavidin, the above-described methods of the invention for producing a device comprise a further step of contacting the coated device with a solution comprising specific capture moieties or capture-associated oligos conjugated with biotin such that said biotinylated capture moieties or capture-associated oligos bind to molecules of avidin or streptavidin immobilized in or adsorbed to the polymer coating of the matrix via a biotin/avidin or biotin/streptavidin binding interaction. Conjugation of biotin with the corresponding capture moieties or capture-associated oligos, a process known to those skilled in the art as biotinylation, can be carried out using procedures well known in the art. Biotinylated peptidic spacers, generally from between 0.4 and 2 nm in length, can also be used to couple the capture moiety or capture-associated oligos to the detection device.

Techniques which allow the conjugation of biotin to a wide range of different molecules are well known in the art. Thus detection electrodes with immobilized avidin or streptavidin can easily made specific for a given target merely by binding of the appropriate biotinylated receptors. Other similar members of binding pair are intended to be within the scope of the present invention, and use of such will be known to one skilled in the art upon reading the present disclosure.

The use of adaptor molecules on the polymer layer considerably improves the reliability of the results obtained during analysis by reducing non-specific interactions of the components of the sample during contact with the matrix, due to the blocking of the free surface of a polymer by adaptor molecules. The use of adaptor molecules also increases the technical efficiency of the device manufacturing process, for example by eliminating the need for an additional surface blocking procedure.

In certain preferred embodiments, the tracking component is an RFID tag. The RFID tag can be encoded with any information deemed useful for the particular applications to which it will be employed, including but not limited to a unique identifying number that can be associated with one or more specific capture moieties, and that are associated with one or more particular target agents. Examples of other information that may be encoded include, inter alia, date of manufacture, lot or batch number, site of manufacture, operator, technician, source of materials, and the like. In some embodiments, the RFID tag is not encoded with any information, and the inherent properties of the tag itself may be used for tracking.

Moreover, information from the devices of the invention can be combined with other information to provide a complete profile of the usage of the device, e.g., in diagnostic applications. In such an embodiment, data regarding biological samples can be linked to the device, for example, by user input, by a scan of a barcode, by a separate RFID tag, or other identifying means associated with the biological sample, e.g., information from LIMS software/databases. The biological sample information can be used to provide comprehensive data regarding the source, inventory and tracking of the biological sample. Such information can be useful in preparing FDA submissions and clinical trial results. In addition, information regarding sample processing, multi-step biological research protocols (assays) performed, production processes, screening, reagents, patient histories, clinical trial data, and other information may be added. The data associated with the devices can be transmitted and shared through a secure hierarchical software and networking architecture that enables interfacing of multi-user, multi-site environments.

In certain embodiments, an RFID tag may be attached to or associated with a substrate such as a plate, microtiter plate, multi-well plate, chip, test tube, probe, flask or any other substrate or suitable reaction container. In certain embodiments an RFID tag may be freely mobile. In other embodiments, a plurality of RFID tags may be connected with other RFID tags (directly or indirectly) to form an array.

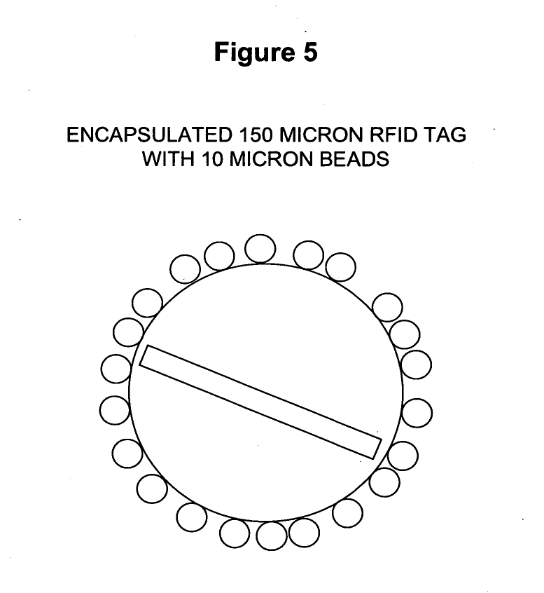

FIG. 5 illustrates an embodiment where a coated reacted loaded RFID complex has been mixed with binding partners that have been immobilized on microspheres. In such an embodiment, the microspheres may confer differences in one or more properties of the reacted loaded RFID complex to allow for the separation of unreacted loaded RFID complexes from reacted loaded RFID complexes. Such properties include but are not limited to size, shape, density, electrical charge or magnetism. Separation means for such reacted loaded RFID complexes include field flow fractionation, centrifugation, electrophoresis, electromagnetism, and the like. In other embodiments, the microspheres may selectively enable RFID interrogation, such that reacted loaded RFID complexes are detectable by RFID interrogation while unreacted loaded RFID complexes are not.

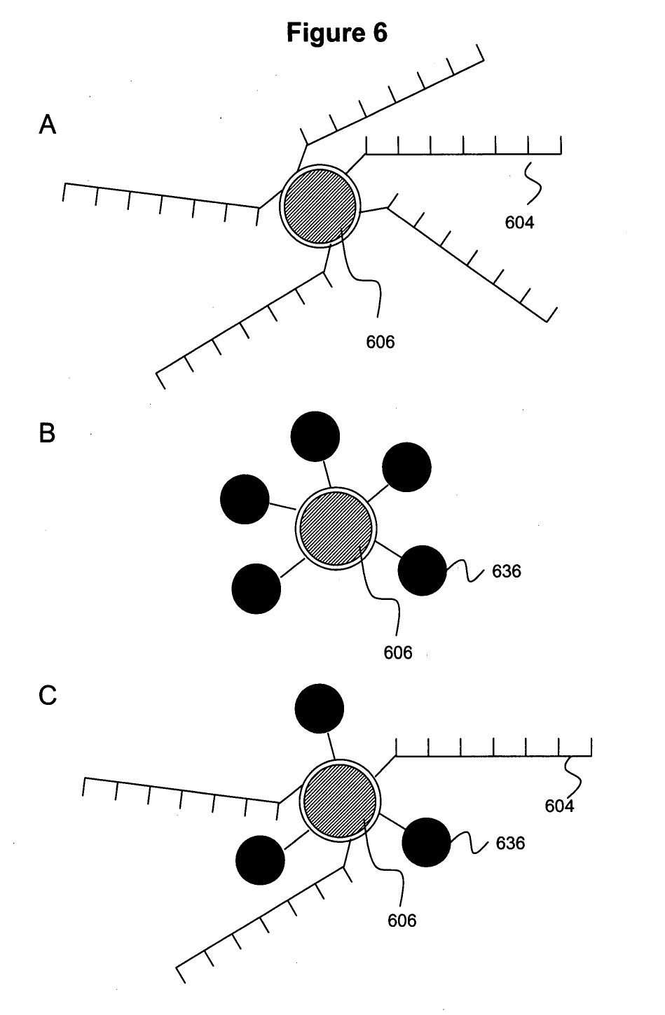

In certain embodiments, an RFID device comprises elements other than or in addition to one or more capture moieties. For example, FIG. 6 depicts various RFID complexes which may be used in various embodiments of the present invention. In A, a plurality of oligonucleotides (604) are affixed to or otherwise attached to an RFID tag (606). In B, a plurality of reactive groups (636) are affixed to or otherwise attached to an RFID tag (606). In C, oligonucleotides (604) and reactive groups (606) are affixed to or otherwise attached to an RFID tag (606).

Once a device of the present invention is contacted with a sample suspected of containing one or more target agents, target agents within the sample will preferentially bind to the capture moieties specific therefore. Binding of a target agent to a capture moiety to create a “reacted capture moiety” facilitates isolation and detection of the device comprising the reacted capture moiety. For example, the reaction mixture comprising the reacted capture moiety can be further contacted with an immobilized binding partner that preferentially binds a different epitope on the target agent than is bound by the capture moiety or that preferentially binds the target agent/capture moiety complex.

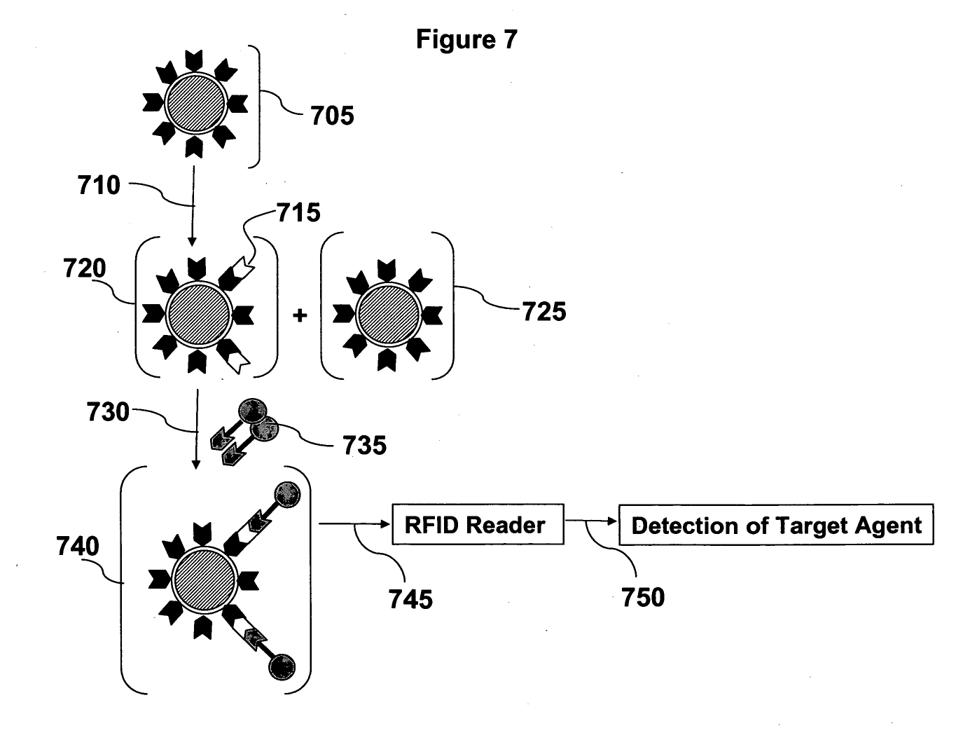

By way of example, and without limiting the scope of the invention, a reaction mixture containing reacted (i.e., bound to target agent) and unreacted (i.e., not bound to target agent) loaded RFID complexes comprising RFID tags and capture moieties can be contacted with a binding partner, as depicted in FIG. 7. In step 710, loaded RFID complexes (705) are introduced to a sample suspected of containing a target agent (715) to form a mixture under conditions to promote binding of the capture moieties to the target agent in the sample, thereby producing reacted loaded RFID complexes (720) and unreacted loaded RFID complexes (725) (e.g., due to an excess of loaded RFID complexes relative to target agent in the mixture). In step 730, the mixture is incubated with an immobilized binding partner (735) that binds selectively to the reacted loaded RFID complexes (e.g., to the target agent/capture moiety complex or to an epitope on the target agent different from the binding site of the capture moiety), thereby immobilizing the reacted loaded RFID complexes and leaving the unreacted loaded RFID complexes in solution. The resulting immobilized reacted loaded RFID complexes (740) are interrogated with an RFID reader at step 745. Analysis of the RFID reader output is used to determine if the target agent was present in the sample at step 750. Specifically, detection of a particular RFID tag in the immobilized complexes is indicative of the presence of a specific capture moiety and, thus, a specific target agent in the sample. In such instances where there is no target agent in the sample, the foregoing immobilized reacted loaded RFID complex does not form and, therefore, no RFID tag is retained for detection. In certain embodiments where it is preferred that a target agent not come into contact with an RFID reader, an RFID tag may be separated from a target agent prior to interrogation. In some embodiments, such separation releases the RFID tag into solution, retaining the target agent in an immobilized phase, and the solution containing the RFID tag but lacking the target agent is subjected to RFID interrogation. In other embodiments, such separation releases the target agent into solution, retaining the RFID tag in an immobilized phase, and the immobilized phase containing the RFID tag but lacking the target agent is subjected to RFID interrogation.

The immobilized binding partner may be conjugated or otherwise immobilized on a substrate such as a bead that allows for isolation of the bound complex by techniques such as centrifugation, size exclusion chromatography, affinity chromatography, ion exchange chromatography, HPLC, FPLC, magnetic capture, electrophoresis, dialysis, filtration, and the like. In a method using magnetic beads, the separation step can be achieved by applying a magnetic field to the magnetic beads. The use of magnetic beads is well known in the art and such beads are commercially available from such sources as Ademtech Inc. (New York, N.Y.), Invitrogen (San Diego, Calif.), Bioclone Inc (San Diego, Calif.) and Promega U.S. (Madison, Wis.). Magnetic beads typically range in size from 50 nm to 20 μm in diameter. The magnetic core of the beads may be encapsulated by a polymer shell, and further modified by surface chemistry to assist the immobilization of molecules such as antibodies to serve as immobilized binding partners on the bead. Magnetic beads may be physically manipulated via the application of a magnetic field which will draw the magnetic beads toward the field, and immobilize them, for instance, on the wall of a test tube adjacent to the magnetic field. Accordingly, with the magnetic beads immobilized, molecules not attached to the magnetic beads may be separated by such methods as aspiration.

In some embodiments, such as that described above, immobilized binding partners will bind to the reacted target agent/capture moiety or to an epitope of the target agent not bound by the capture moiety (i.e. reacted loaded RFID complexes), leaving the unreacted loaded RFID complexes in solution. The substrate comprising the immobilized binding partner, and therefore the reacted loaded RFID complexes, may be subjected to RFID interrogation to detect/identify the RFID tags thereon, thereby identifying target agents from the sample. Alternatively, the reacted loaded RFID complexes may be removed from the substrate prior to RFID interrogation, for example, subsequent to removal of the unreacted loaded RFID complexes from the mixture, e.g., by aspiration and/or washing the substrate. In other embodiments, immobilized binding partners will bind to unreacted loaded RFID complexes (e.g. to unreacted capture moieties)), leaving the reacted loaded RFID complexes in solution and available for hybridization to, e.g., a biosensor, or for application to an RFID reader.

For example, in certain embodiments of the present invention antibodies are conjugated to a device comprising an RFID tag to form a loaded RFID complex, and the target agent of interest is an antigen. In some such embodiments, the following elements are included, in varied orders or combinations: (1) an RFID tag associated with a capture moiety comprising an antibody corresponding to a target agent to form a loaded RFID complex, (2) immobilized binding partners, and (3) a sample suspected of containing the target agent. In one aspect, the loaded RFID complex is contacted with the sample to form a first mixture, and the first mixture is contacted with an excess of immobilized binding partners, which specifically bind to capture moieties that have not bound target agent (e.g., the same antigen as the target agent). The unreacted loaded RFID complexes are captured by the immobilized binding partners and the reacted loaded RFID complexes that have bound to target agent from the sample are left in solution, thereby separating the unreacted loaded RFID complexes from the reacted loaded RFID complexes. The RFID tags from the reacted loaded RFID complexes are scanned with an RFID reader to determine if RFID tags remain in solution, which is an indication that target agents were in the sample. Alternatively, the reacted loaded RFID complexes can be immobilized with immobilized binding partners specific to the reacted loaded RFID complexes, e.g., at a different portion (e.g., epitope) of the target agent (antigen) or to the target agent/capture moiety (antigen/antibody) complex, thereby immobilizing the reacted loaded RFID complexes and leaving the unreacted loaded RFID complexes in solution where they can be removed by, e.g., decanting, washing, etc.

Some embodiments are employed in a multi-target (multiplexed) format, allowing for the screening of multiple target agents simultaneously. Such embodiments include providing, in varied orders or combinations: (1) a set of loaded RFID complexes, comprising at least one loaded RFID complex specific for each of a plurality of target agents, (2) immobilized binding partners specific for the capture moieties on the loaded RFID complexes (e.g., comprising the same epitopes of the target agents that are bound by the capture moieties), and (3) a sample suspected of containing one or more target agents. The method comprises mixing/contacting the sample with the set of loaded RFID complexes under reaction conditions that allow the capture moieties on the loaded RFID complexes to capture target agent present in the sample to form a first mixture. The first mixture is mixed/contacted with an excess of immobilized binding partners specific for the capture moieties that have not bound target agent. The capture moieties on the loaded RFID complexes that have not reacted with target agents in the sample (unreacted loaded RFID complexes) react with the immobilized binding partners to form an immobilized phase. The solution phase comprises the loaded RFID complexes that have reacted with target agents in the sample (reacted loaded RFID complexes). The solution phase is separated from the immobilized phase and the RFID tags from the reacted loaded RFID complexes are then scanned with an RFID reader (interrogated) to determine which RFID tags are present, e.g., according to the identification numbers of the RFID tags in solution. The RFID tags present in solution are indicative of which target agents are present in the sample. Alternatively, the reacted loaded RFID complexes can be captured by the immobilized binding partners (e.g., through specific binding to a) the target agents at different epitopes than those bound by the capture moieties, or b) the target agent/capture moiety complex), thereby leaving the unreacted loaded RFID complexes in solution. The immobilized phase is separated, the unreacted loaded RFID complexes discarded, and the reacted loaded RFID complexes are interrogated. In some embodiments, the same RFID tags are used for different capture moieties, and in other embodiments, different RFID tags are used for the different capture moieties.

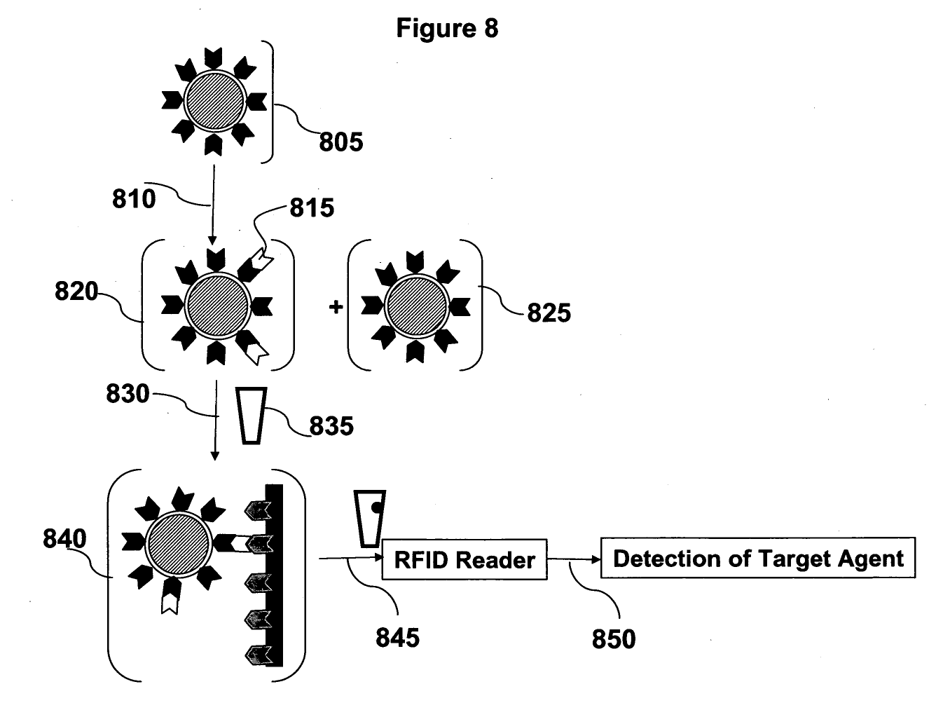

In another example, the mixture containing the unreacted loaded RFID complexes and reacted loaded RFID complexes can be contacted with a vessel to which the binding partners have been immobilized (FIG. 8). At step 810, the loaded RFID complexes (805) are introduced to a sample suspected of containing a target agent (815) under conditions to promote binding of the capture moieties of the loaded RFID complexes to the sample, thereby producing reacted loaded RFID complexes (820) and unreacted loaded RFID complexes (825) (e.g., due to an excess of loaded RFID complexes relative to target agent in the mixture or to an absence of target agent in the sample). In step 830, the mixture is incubated with an immobilized binding partner in a vessel (835) that binds selectively to the reacted loaded RFID complexes (either to the target agent/capture moiety complex or to an epitope on the target agent different from the binding site of the capture moiety), thereby immobilizing the reacted loaded RFID complexes to the vessel surface, effecting their capture. The unreacted loaded RFID complexes remain in solution and are removed from the vessel, e.g., through a rinsing/washing step. The immobilized reacted loaded RFID complexes (840) retained in the vessel are subjected to RFID interrogation at step 845. Analysis of the RFID reader output is used to determine if the target agent was present in the sample at step 850. Specifically, detection of a particular RFID tag in the immobilized complexes is indicative of the presence of a specific capture moiety and, thus, a specific target agent in the sample. In such instances where there is no target agent in the sample, the foregoing immobilized reacted loaded RFID complex does not form and, therefore, no RFID tag is retained for detection.

In an alternative embodiment, the mixture containing the unreacted loaded RFID complexes and reacted loaded RFID complexes are contacted with a column comprising the immobilized binding partner(s). In some such embodiments, the reacted loaded RFID complexes are immobilized by binding to the immobilized binding partners, allowing any remaining solution phase reagents (which includes the unreacted loaded RFID complexes) to be removed. The column may then be subjected to RFID interrogation, or the bound complexes may be removed from the column before subjecting them to RFID interrogation. In other such embodiments, the unreacted loaded RFID complexes are immobilized by binding to the immobilized binding partners, allowing any remaining solution phase reagents (which includes the reacted loaded RFID complexes) to pass through the column for collection and subsequent RFID interrogation.

In certain embodiments, the reaction is multiplexed by the use of multiple devices, each with a different capture moiety conjugated thereon. In this manner, multiple target agents can be screened and detected in a single reaction. For example, in multiplexed embodiments using an RFID device, different RFID frequencies can be employed for each particular RFID tag (and, therefore, each capture moiety), allowing the reporting of multiple different signals when interrogated. Alternatively, RFID tags with unique identification information (“identifiers”) may be used to distinguish between multiple RFID tags with different capture moieties.

In some embodiments, multiplexing can be accomplished through the use of different immobilized binding partners at known locations on a substrate. An RFID tag immobilized on a substrate at a location known to contain a particular immobilized binding partner is indicative that the target agent specifically bound by the particular immobilized binding partner is present. For example, the ability of one or more cell receptors to bind to one or more antagonists can be tested by affixing many different potential antagonists to known locations on surface, and allowing cell receptors with RFID tags attached to bind to the antagonists. The presence of an affinity reaction between a particular cell receptor and a particular antagonist can be determined by scanning the surface for the presence and identity of an RFID tag, and then correlating that RFID tag with the location on the array to which it was found. In this manner, potential affinity reactions between multiple cell receptors and multiple antagonists can be analyzed in a single reaction.

In another related example, a sample suspected of containing target agents A, B, and C is mixed with loaded RFID complexes, each comprising either capture moiety A′ (specific for A), B′ (specific for B), or C′ (specific for C) under conditions that promote specific binding between the target agents and the capture moieties specific therefore. The mixture is exposed to a matrix comprising immobilized binding partners A″, B″, and C″, each of which is specific for target agent A, B, or C, respectively. (Alternatively, the immobilized binding partners may be specific for the corresponding target agent/capture moiety complex.) Further, each of the immobilized binding partners is affixed to a known location on the matrix. Association of the reacted loaded RFID complexes (i.e., loaded RFID complexes bound to target agent) to the immobilized binding partners results in creation of immobilized reacted loaded RFID complexes, each at a location known to comprise the corresponding immobilized binding partner. The matrix is scanned by an RFID reader and the location of any RFID tags present is determined. Since each immobilized binding partner has a known location and is specific for a single target agent (or target agent/capture moiety complex), the location of an RFID tag on the matrix is an indication that the target agent was present in the sample. For example, if target agents A and B were present in the sample, and target agent C was absent from the sample, then locations on the matrix corresponding to immobilized binding partners A″ and B″ would contain RFID tags, while locations on the matrix corresponding to immobilized binding partner C″ would not contain RFID tags. In such embodiments, the RFID tags in the RFID complexes may be identical to one another, or may be distinguishable from one another, for example, based on information encoded therein. For example, an RFID tag in an RFID complex with a capture moiety A′ may be encoded with that information, which can be used to confirm that immobilization of the RFID tag on the substrate is specific, i.e., that it is immobilized at immobilized binding partner A″, which is specific for target agent A.

In certain embodiments, a matrix comprising a plurality of immobilized binding partners is used. In such a matrix, the immobilized binding partners may all be identical (e.g., to provide for internal controls or replication to further validate the detection of a target agent in a sample), different (e.g., for detection of multiple target agents in a sample), or may comprise a combination of identical and different immobilized binding partners. In certain embodiments, different sections of a matrix contain multiple immobilized binding partners, each specific for the same target agent (or target agent/capture moiety complex), and in other embodiments such identical immobilized binding partners are distributed across the matrix, e.g., to control for any surface characteristics that may affect scanning of the matrix.

For example, in one particular embodiment a matrix comprises several sets of immobilized binding partners. Each set comprises different immobilized binding partners specific to different strains of a given species of pathogenic microorganism, and there is a different set of immobilized binding partners for each pathogenic microorganism of interest. The matrix is divided into sections, each of which contains all the immobilized binding partners from a given set at known locations. A sample suspected of containing a pathogenic microorganism is mixed with loaded RFID complexes comprising capture moieties specific for the same strains of microorganisms as are the immobilized binding partners on the matrix, and the resulting mixture is added to the matrix under conditions that promote binding of the immobilized binding partners to the reacted loaded RFID complexes (e.g., by binding a different portion of a target agent (e.g., cell surface receptor) than the capture moiety, or by binding a target agent/capture moiety complex). Only the RFID tags in reacted loaded RFID complexes will be immobilized by binding to the immobilized binding partners on the matrix, and the solution phase containing unreacted loaded RFID complexes can be removed. Scanning the matrix with an RFID reader identifies the locations at which there is an RFID tag, thereby identifying which, if any, pathogenic microorganism is present in the sample based on which immobilized binding partner is known to be at that location. In such an embodiment, the loaded RFID complexes may comprise, e.g., a plurality of identical capture moieties specific for a single strain of microorganism, or may comprise a set of capture moieties specific for different strains of a single species of microorganism.

In certain embodiments where multiple steps are involved in the analysis of a sample, the reaction mixes at any step may be read by an RFID reader as the analysis proceeds through the steps in order to monitor the reaction (e.g., track the location of the RFID tags). For instance, where a procedure involves a washing step, the reaction mix may be scanned with an RFID reader both before and after the washing step to determine which of the RFID tags were removed in the washing step and which were retained after the washing step. Comparison of the RFID tags present before and after the wash step can indicate, for example, the efficiency of the wash step.

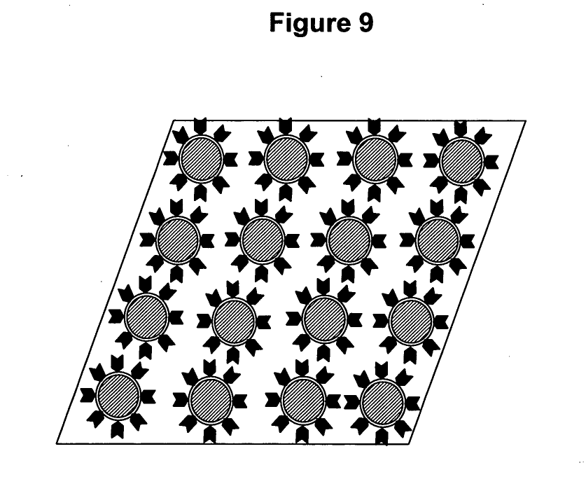

In certain embodiments (as illustrated in FIG. 9), RFID devices are associated with a planar matrix to form an RFID microarray. An RFID microarray is exposed to a sample suspected of containing a target agent. Binding of the target agent to a capture moiety on one of the RFID devices changes one or more characteristics of the RFID tag associated therewith, and the one or more characteristic changes are detectable by RFID interrogation. As such, RFID interrogation of the RFID microarray reveals which RFID device has bound target agent, thereby identifying the target agent in the sample. When used in this fashion, each RFID microarray can be used to scan for multiple target agents simultaneously, and the planar construction is consistent with current industry uses. In addition, an additional tracking component can be associated with the matrix of the array itself (such as an additional RFID tag or barcode) to a) provide an indication of the identity of the RFID device and, additionally, b) cue the device to query the user for confirmation that the assay being used for the sample preparation is appropriate for the particular array, c) identify one or more reagents to be employed with the particular array, or d) provide other information to ensure that the correct analysis is performed.

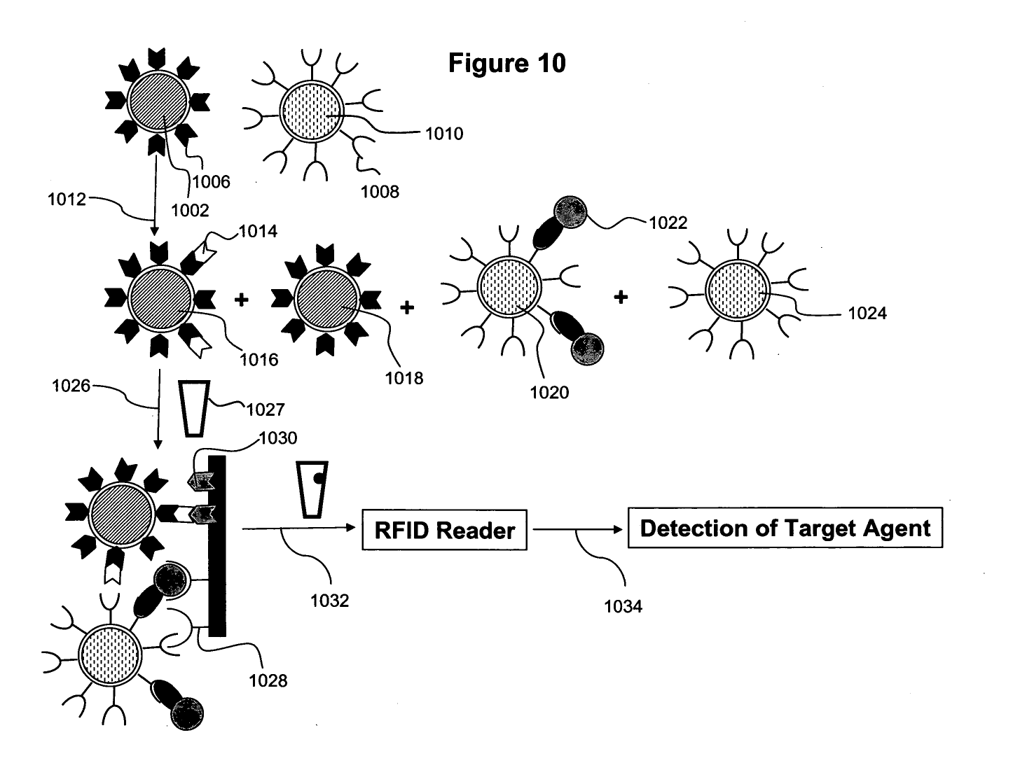

In another example, different target agents in a sample can be detected using multiple loaded RFID complexes (FIG. 10). Multiple loaded RFID complexes (1002 and 1010) with different capture moieties (1006 and 1008) can recognize different target agents. As described above, the loaded RFID complexes are introduced to a sample suspected of containing a target agent (step 1012) under conditions to promote binding of the capture moieties of the loaded RFID complexes to the sample. A first reacted loaded RFID complex (1016) has a first target agent (1014) bound to its capture moieties, and a second reacted loaded RFID complex (1020) has a second target agent (1022) bound to its capture moieties. Unreacted loaded RFID complexes (1018 and 1024) have no target agent bound to their capture moieties. The mixture is then incubated with immobilized binding partners (1028 and 1030) that bind selectively to their respective capture moiety/target agent complexes and the reacted loaded RFID complexes (1016 and 1020) are immobilized to the surface of vessel (1027), effecting creation of immobilized reacted loaded RFID complexes. The unbound unreacted RFID complexes (1018 and 1024) are removed from the vessel, e.g., through a rinsing/washing step, and the vessel is subsequently subjected to RFID interrogation (step 1032). Identification of one or more particular RFID tags indicates the presence of one or more of the specific capture moieties, thus, specific target agents, in the sample (step 1034). In instances where there is no target agent in the sample, the immobilized reacted loaded RFID complexes do not form and no RFID tags are retained for detection. In instances where only one target agent is in the sample, only one of the immobilized reacted loaded RFID complexes forms, and only one RFID tag is retained for detection.

Certain embodiments of the present invention include biologic receptors affixed to or otherwise associated with an RFID tag to form a loaded RFID complex, and immobilized target agents comprising receptor antagonists. In accordance with these embodiments of the invention the following elements are included, in varied orders or combinations: (1) an RFID tag where the RFID tag is associated with capture moieties comprised of biologic receptors (a loaded RFID complex): and (2) immobilized target agents where the immobilized target agents comprise biologic receptor antagonists. In one aspect, the loaded RFID complex is contacted with the immobilized target agents. The loaded RFID complexes are bound or “captured” by the immobilized biologic antagonists to form reacted loaded RFID complexes, and the unreacted loaded RFID complexes remain unbound. The unreacted loaded RFID complexes are washed or otherwise separated or removed from the reacted loaded RFID complexes. The bound reacted loaded RFID complexes are then scanned with an RFID reader (interrogated) and the reacted loaded RFID complexes are identified, e.g., based on RFID identification numbers. The presence of a reacted loaded RFID complex indicates a binding event between the biologic receptor on that reacted loaded RFID complex and the immobilized biologic receptor antagonist. Alternatively, the biologic receptor antagonists may be associated with the RFID tag to form a loaded RFID complex, and the immobilized target agents may comprise the biologic receptors. Note that in certain alternative embodiments, the RFID tag could instead be any other type of tracking component that may be associated with a capture moiety (e.g., a biologic receptor or biologic receptor antagonist). Such tracking components are well known to those of skill in the art.

In alternative embodiments of the present invention, biologic receptors are conjugated to or otherwise associated with an RFID tag to form a loaded RFID complex, and immobilized target agents comprise biologic receptor agonists. In accordance with these embodiments of the invention the following elements are included, in varied orders or combinations: (1) an RFID tag where the RFID tag is associated with capture moieties comprising biologic receptors (a loaded RFID complex): and (2) immobilized target agents where the immobilized target agents comprise biologic receptor agonists. In one aspect, the loaded RFID complex is contacted with the immobilized target agents. The loaded RFID complexes are bound or “captured” by the immobilized biologic agonists to form reacted loaded RFID complexes, and the unreacted loaded RFID complexes remain unbound. The unreacted loaded RFID complexes are washed or otherwise removed from the reacted loaded RFID complexes. The bound reacted loaded RFID complexes are then scanned with an RFID reader (“interrogated”) and the reacted loaded RFID complexes are identified, e.g., based on RFID identification numbers. The presence of a reacted loaded RFID complex indicates a binding event between the biologic receptor on that reacted loaded RFID complex and the immobilized biologic receptor agonist. Alternatively, the biologic receptor agonists may be associated with the RFID tag to form a loaded RFID complex, and the immobilized target agents may comprise the biologic receptors. Note that in certain alternative embodiments, the RFID tag could instead be any other type of tracking component that may be associated with a capture moiety (e.g., a biologic receptor or biologic receptor antagonist). Such tracking components are well known to those of skill in the art.

In certain embodiments (e.g., those presented above), the immobilized target agents may be immobilized to known locations on a matrix. In this manner, binding events between biologic receptors and biologic receptor agonists/antagonists may be detected in a multiplexed format, allowing for the screening of multiple binding events simultaneously. In such a format and following screening, the presence and location of a reacted loaded RFID complex on the matrix indicates a binding event between that reacted loaded RFID complex and the target agent known to be immobilized that that location on the matrix. For example, an RFID identification number associated with the RFID component of the reacted loaded RFID complex may then be correlated with the location, and hence the identity, of the immobilized target agent to which it is bound. In this manner, a binding event between a capture moiety on a particular loaded RFID complex and a particular immobilized target agent is determined.

In alternative embodiments of the present invention, enzymes are conjugated to an RFID tag to form a loaded RFID complex, and immobilized target agents comprise biologic enzyme substrates. In accordance with these embodiments of the invention, the following elements are included, in varied orders or combinations: (1) an RFID tag where the RFID tag is associated with capture moieties comprised of biologic enzymes and (2) immobilized target agents comprising biologic enzyme substrates. In one aspect, the loaded RFID complexes are contacted with the immobilized biologic enzyme substrates (target agents). The loaded RFID complexes are captured by the immobilized biologic enzyme substrates to form reacted loaded RFID complexes, and the unreacted loaded RFID complexes remain unbound. The unreacted loaded RFID complexes are washed or otherwise removed from the reacted loaded RFID complexes. The bound reacted loaded RFID complexes are then scanned with an RFID reader and are identified, e.g., based on RFID identification numbers. The presence of a reacted loaded RFID complex indicates a binding event between the biologic enzyme on that reacted loaded RFID complex and the immobilized biologic enzyme substrate (target agent). Alternatively, the biologic enzyme substrates may be associated with the RFID tag to form a loaded RFID complex, and the immobilized target agents may comprise the biologic enzymes. Additionally, a biologic enzyme inhibitor may be used in place of, or in conjunction with, a biologic enzyme substrate. Alternatively, the immobilized target agents may be immobilized to known locations on a matrix. In this manner, binding events between biologic enzymes and biologic enzyme substrates/inhibitors may be detected in a multiplexed format, allowing for the screening of multiple binding events simultaneously. In such a format, and following screening, the presence and location of a reacted loaded RFID complex on the matrix indicates a binding event between that reacted loaded RFID complex and the immobilized target agent known to be immobilized that that location on the matrix. For example, an RFID identification number associated with the RFID component of the reacted loaded RFID complex may then be correlated with the location, and hence the identity, of the immobilized target agent to which it is bound. In this manner, a binding event between a capture moiety on a particular loaded RFID complex and a particular immobilized target agent is determined. Note that in certain alternative embodiments, the RFID tag could instead be any other type of tracking component that may be associated with a capture moiety (e.g., a biologic enzyme, biologic enzyme substrate, or biologic enzyme inhibitor). Such tracking components are well known to those of skill in the art.

In yet further embodiments, a reverse RFID capture method is used where the immobilized binding partner is contacted with the sample to form a first mixture, then this mixture is contacted with a loaded RFID complex. Capture moieties on the loaded RFID complexes bind to a different epitope of the target agent than that bound by the capture moiety, or to the target agent/immobilized binding partner complex to create immobilized reacted loaded RFID complexes. Loaded RFID complexes that do not bind to the immobilized target agent/immobilized binding partner complex (unreacted loaded RFID complexes) remain in solution. Detection of RFID tags in the immobilized reacted loaded RFID complexes proceeds as described elsewhere herein. Other variations on this embodiment include one or more other aspects of the invention described herein or such other modifications known to those of ordinary skill in the art.

For example, in some embodiments of the reverse RFID capture method the target agent of interest is an antibody, the immobilized binding partner is an antigen, and the loaded RFID complex has capture moieties that recognize the target agent/immobilized binding partner complex. In accordance with this embodiment of the invention the following elements are included, in varied orders or combinations: (1) a loaded RFID complex comprising an RFID tag and a capture moiety comprising an antibody corresponding to the target agent/binding partner complex; (2) immobilized binding partners comprising antigens to the target antibody; and (3) a sample suspected of containing the target antibody. In one aspect, the sample is contacted with the immobilized binding partners to form a mixture, and the mixture is contacted with the loaded RFID complexes. The loaded RFID complexes that bind to target agent/binding partner complexes are immobilized. The unreacted RFID complexes stay in the solution phase of the mixture, and can be separated from the immobilized phase by techniques known in the art. The RFID tags from the immobilized reacted loaded RFID complexes are scanned with an RFID reader to determine which RFID tags are present, e.g., using their encoded identification numbers, and thereby determining which target agents are present in the sample.

In certain embodiments, the reverse RFID capture method may be used to analyze multiple targets in a sample using a multiplexed) format. For example, immobilized binding partners are contacted with the sample to create a first mixture, and the first mixture is contacted with loaded RFID complexes in a reverse RFID capture scenario Such an embodiment includes providing, in varied orders or combinations: (1) a set of loaded RFID complexes comprising at least one loaded RFID complex specific for each of a plurality of target agents, wherein each loaded RFID complex comprises an RFID tag and at least one capture moiety that binds to a) a different portion of a given target agent than the immobilized binding partner or b) to the target agent/binding partner complex; (2) immobilized binding partners that specifically bind the plurality of target agents; (3) and a sample suspected of containing at least one of the target agents. The method comprises mixing/contacting the sample with the immobilized binding partners under reaction conditions that allow the immobilized binding partners to capture target agents in the sample to form a first mixture. The first mixture is mixed/contacted with the loaded RFID complexes under conditions to promote association of the loaded RFID complexes with immobilized target agent or target agent/binding partner complexes and, consequently, immobilization of reacted loaded RFID complexes. The loaded RFID complexes that do not bind to the immobilized target agent or target agent/binding partner complexes will remain in solution. The immobilized phase is separated, and the reacted loaded RFID complexes are released into solution and scanned with an RFID reader to reveal which RFID tags are associated with the reacted loaded RFID complexes, and, therefore, which target agents are in the sample.

In some embodiments, tracking components (e.g., RFID tags) are associated with oligonucleotides for the purpose of detecting one or more nucleic acid target sequences, e.g., single nucleotide polymorphisms (SNPs), in nucleic acid sequences. Such oligonucleotides may be referred to as “capture oligos.” Detection of SNPs is important because sequence differences in DNA between individuals may explain differences, e.g., in disease resistance, disease susceptibility, and drug response. Methods for sequencing DNA are well known in the art. See, for example, Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), hereby incorporated by reference.

Oligonucleotides used in SNP detection are often designed to be complementary to the nucleic acid sequence of a target nucleic acid, with the exception of the variable position (SNP) of the oligonucleotide. For the detection of a SNP at a particular position, the variable position of the oligonucleotides (often the middle or internal position) may contain all possible nucleotide permutations at that position. For example, if the target nucleic acid is DNA, there may be one oligonucleotide with an “A” deoxyribonucleotide at the variable position, a second oligonucleotide with an “T” deoxyribonucleotide at the variable position, a third oligonucleotide with an “C” deoxyribonucleotide at the variable position, and a fourth oligonucleotide with an “G” deoxyribonucleotide at the variable position. If the target nucleic acid has a “T” deoxyribonucleotide at the location corresponding to the variable position on the oligonucleotide, it will preferentially bind to the oligonucleotide with the “A” deoxyribonucleotide at the variable position. Use of additional oligonucleotides can facilitate further analysis, e.g., statistical analysis related to background and/or quality control for the genotyping result.

Prior to hybridization, double-stranded target nucleic acid(s) may be converted to single-stranded target nucleic acids using methods such as heating the target nucleic acid to 95° C. for 1 minute. The target nucleic acid(s) may also be fragmented into small sections using methods well known in the art such as treatment with restriction endonucleases. A “restriction endonuclease” is any enzyme capable of recognizing a specific sequence (the “restriction site”) on a double- or, in some embodiments, single-stranded polynucleotide and cleaving the polynucleotide at or near the site. Examples of site-specific restriction endonucleases are available in the 2006 New England Biolabs, Inc. catalog, including the 2006 New Products Catalog Supplement, which is incorporated herein by reference.

The hybridization reaction between an oligonucleotide (e.g., capture oligo or other oligonucleotide) and a target nucleic acid is typically performed in solution where the metal ion concentration of the buffer is between 0.01 mM to 5 M and a pH range of pH 5 to pH 10. Other components can be added to the buffer to promote hybridization such as dextran sulfate, EDTA, surfactants, etc. The hybridization reaction can be performed at a temperature within the range of 10° C. to 90° C., preferably at a temperature within the range of 25° C. to 60° C., and most preferably at a temperature within the range of 30° C. to 50° C. Alternatively, the temperature is chosen relative to the melting temperatures of the nucleic acid molecules employed. The reaction is typically performed at an incubation time from 10 seconds to about 12 hours, and preferably an incubation time from 30 seconds to 5 minutes. A variety of hybridization conditions may be used in the present invention, including high, moderate and low stringency conditions; see for example Maniatis et al., Molecular Cloning: A Laboratory Manual, 3rd Edition (2001), hereby incorporated by reference. Persons of ordinary skill in the art will recognize that stringent conditions are sequence-dependent and are dependent upon the totality of the conditions employed. Longer sequences typically hybridize specifically at higher temperatures. Generally, stringent conditions are selected to be about 5-10° C. lower than the Tₘ for the specific sequence at a defined ionic strength pH. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

The target nucleic acid sequence may be generated using sequencing methods well known in the art. See, for example, Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), hereby incorporated by reference. Nucleic acid sequencing may also be automated with machines such as the ABI Catalyst and 373 and 377 DNA Sequencers (Perkin Elmer, Wellesley, Mass.). The sequence may also be obtained from publicly-available and/or custom databases. Custom databases may be databases populated with information from publicly-available databases. Major publicly-available sequence repositories include DDBJ: DNA databank of Japan, EMBL: maintained by EMBL, and GenBank: maintained by NCBI.

In embodiments where an oligonucleotide is immobilized on a matrix, such immobilization may be accomplished directly or indirectly by covalent bonding, ionic bonding, physical adsorption, and other methods well known in the art. Examples of immobilization by covalent bonding include a method in which the surface of the matrix is activated and the nucleic acid molecule is then immobilized directly to the matrix or indirectly through a cross-linking agent. Yet another method using covalent bonding to immobilize an oligonucleotide includes introducing an active functional group into an oligo followed by direct or indirect immobilization. The activation of the surface may be conducted by electrolytic oxidation in the presence of an oxidizing agent, or by air oxidation or reagent oxidation, as well as by covering with a film. Useful cross-linking agents include, but are not limited to, silane couplers such as cyanogen bromide and gamma-aminopropyl triethoxy silane, carbodiimide and thionyl chloride, and the like. Useful functional groups to be introduced to the oligo may be, but are not limited to, sulfide, disulfide, amino, amide, amido, carboxyl, hydroxyl, carbonyl, oxide, phosphate, sulfate, aldehyde, keto, ester, and mercapto groups. Other highly reactive functional groups may also be employed using methods readily known to those of ordinary skill in the art.

Oligonucleotides for use in the methods disclosed herein can be 1 to 10,000 bases in length, preferably 10 to 1000 bases in length, more preferably 10-500 bases in length. In some embodiments, the oligonucleotides are about 25 to about 100 bases in length. Additionally, the oligonucleotides may be DNA, RNA or PNA (peptide nucleic acid) or any chemically-modified variant thereof, or combinations thereof, and can include non-naturally occurring subunits, sequences and/or moieties. PNA includes peptide nucleic acid analogs. The backbones of PNA are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched base pairs. DNA and RNA typically exhibit a 2-4° C. drop in Tₘ for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This is advantageous, as a reduced salt hybridization solution has a lower Faradaic current than a physiological salt solution (in the range of 150 mM).

In certain embodiments, oligonucleotides may be amplified by methods known to those of skill in the art. Briefly, in some embodiments that include an amplification step, a capture-associated oligo is used as a template for linear amplification, and the capture-associated oligo is therefore designed to encode a) a sequence identical to a sequence of the corresponding electrode-associated oligo (as opposed to a sequence complementary to a sequence of the electrode-associated oligo, as would be the case if the capture-associated oligo were to be hybridized directly to the electrode-associated oligo), and b) a sequence corresponding to a polymerase recognition sequence at its 3′ end adjacent to or overlapping with the region identical to a sequence of the electrode-associated oligo. In other embodiments, a logarithmic amplification technique may be used to amplify oligos in conjunction with the present invention. Such methods are known to those of skill in the art and include, but are not limited to, polymerase chain reaction (PCR) techniques. PCR may be carried out using materials and methods well known to those of skill in the art, as are the many modifications to the basic method such as variations in the polymerase, reaction buffer, template nucleic acid, thermal cycling profile, reaction additives, primer design and other modifications.

In some embodiments of the invention, oligonucleotides may be separated from a molecule or complex, such as an RFID complex, prior to further manipulation or analysis, e.g., amplification, labeling, hybridization, and/or detection. Briefly, some methods include a separation (via e.g., cleavage, degradation, etc.) of capture-associated oligos from reacted loaded RFID complexes, e.g., following separation of complementary oligos, e.g., electrode-associated oligos. For example, such separation can be useful when reacted capture-associated oligos are conjugated to a loaded RFID-oligo complex that interferes with hybridization or electrochemical detection, e.g., because of the physical size or the presence of local areas of electron density on the surface of the loaded RFID-oligo complex. Separation can be achieved, for example, by using a digestive enzyme or an enzyme that causes hydrolysis of a bond in a molecule (e.g., proteolytic enzymes, lipases, phosphatases, phosphodiesterases, esterases, etc.), endonucleases (specific for single-stranded or double-stranded sequences), exonucleases, a restriction endonuclease (e.g., EcoRI, HaeIII), or a flap endonuclease (e.g., FEN-1, RAD2, XPG, etc.). The choice of separation method will depend on the nature of the components of the loaded RFID-oligo complex and its conjugation to the capture-associated oligo. Those of skill in the art will readily appreciate and understand the circumstances under which one particular method of separation would be preferred over another method of separation. In certain embodiments, such separation methods are combined with amplification methods, such as those described above.

In some embodiments, methods are provided for analyzing a target nucleic acid, including, for example, determining its presence in a sample, determining its nucleic acid sequence, genotyping a SNP position, and/or detecting a nucleic acid. Such methods include, in varying orders and combinations, a) mixing a first complex comprising a tracking component capable of generating a signal and a first nucleic acid with a sample suspected of containing a target nucleic acid capable of forming a nucleic acid duplex with the first nucleic acid to form a second complex; b) contacting the second complex with a moiety capable of affecting the signal; and c) analyzing the target nucleic acid by detecting an effect imparted by the moiety on the signal. In certain specific embodiments, the moiety is a nucleic acid-binding protein, an intercalating agent, a metallo complex, a cis-platen, a heme compound, a ruthenium-containing compound, a platinum-containing compound, an iron-containing compound, a transition metal-containing compound, or a combination or plurality thereof. In certain embodiments, the effect imparted by the moiety comprises enabling the tracking component to display the signal or otherwise altering the signal. For example, the signal may be altered by the moiety relative to a baseline signal generated in the absence of the moiety by, e.g., increasing, decreasing, enhancing, or altering its frequency or wavelength. In addition, in some embodiments, the first complex is immobilized on a matrix. In some embodiments, the moiety is, or is associated with, an immobilized binding partner.

Specific embodiments of methods for genotyping one or more SNPs in a nucleic acid sample using tracking components (e.g., RFID tags) with associated capture oligos include the use of in varied orders or combinations: (1) a sample containing target nucleic acid (e.g., fragmented target nucleic acid), and (2) a set of capture oligos for each SNP to be genotyped, each of which is affixed or otherwise associated to an RFID tag to create a set of RFID-oligo complexes, where the set comprises capture oligos complementary to the target nucleic acid and identical to one another except at the SNP position, where different subsets of the capture oligos contain different nucleotides at the SNP position, such that the set of capture oligos on the matrix contains at least one capture oligo for each possible nucleotide (e.g., A, T, G, or C) at the SNP position. For example, there may be four different subsets of capture oligos in a set: one comprised of capture oligos with an A at the SNP position, one comprised of capture oligos with a T at the SNP position, one comprised of capture oligos with a G at the SNP position, and one comprised of capture oligos with a C at the SNP position. In other embodiments, only two subsets of capture oligos may be used, one complementary to each of the known genotypes for the SNP position. In further embodiments, the capture oligos complementary to all subsets of the aforementioned capture oligos may also be in RFID-oligo complexes. In addition, each different capture oligo is complexed with a different RFID tag, so that identification of a particular RFID tag in a detector is indicative of a particular capture oligo, and therefore a particular target nucleic acid.

Some methods include mixing the target nucleic acid with the RFID-oligo complexes under conditions that permit hybridization of target nucleic acid fragments to capture oligos perfectly complementary thereto to create a first mixture. Where the SNP position of the capture oligos is not perfectly complementary to the target nucleic acid, hybridization does not typically occur. The first mixture is introduced to immobilized binding partners, which facilitate the separation of reacted RFID-oligo complexes (i.e., RFID-oligo complexes bound to target nucleic acid) from unreacted RFID-oligo complexes (i.e., RFID-oligo complexes not bound to target nucleic acid). For example, the immobilized binding partners may be oligonucleotides complementary to capture oligos on the RFID-oligo complexes and the first mixture may be introduced to them under conditions that promote binding of the capture oligo on the RFID-oligo complex to the immobilized oligonucleotide. Only unreacted RFID-oligo complexes are immobilized, and the reacted RFID-oligo complexes are recovered, e.g., by decanting and/or washing. The solution phase containing the reacted RFID-oligo complexes is scanned with an RFID reader and the RFID tags in the reacted RFID-oligo complexes are identified. An RFID tag detected in the solution phase is indicative of a particular capture oligo in the solution phase, and the genotype of the SNP is determined based on the sequence of the capture oligo detected in the solution phase. In this manner, the genotype (or genotypes, e.g., if the sample is from a heterozygous organism) of the SNP in the sample is provided. In certain embodiments, multiple SNP positions can be genotyped simultaneously in this manner.

In alternative embodiments, immobilized binding partners can be designed to immobilize reacted RFID-oligo complexes, e.g., through specific binding to (a) a region of the target nucleic acid that is different than the region bound by the capture oligo on the RFID-oligo complex (e.g., a region adjacent to the region bound to the RFID-oligo complex), or (b) the duplex formed by binding of the target nucleic acid to the capture oligo. In such an embodiment, reacted RFID-oligo complexes (i.e., RFID-complexes bound to target nucleic acid) are immobilized and unreacted RFID-oligo complexes (i.e., RFID-complexes not bound to target nucleic acid) may be removed by, e.g., decanting, washing, etc. The reacted RFID-oligo complexes may be interrogated while immobilized or subsequent to release from immobilization by methods well known in the art. In certain embodiments, binding of an immobilized binding partner to a reacted RFID-oligo complex affects a signal from the RFID tag, for example, by increasing, decreasing, enhancing, or otherwise altering it (e.g., by changing its wavelength or frequency). In some embodiments, the immobilized binding partner is a nucleic acid-binding protein, an intercalating agent, a metallo complex, a cis-platen, a heme compound, a ruthenium-containing compound, a platinum-containing compound, an iron-containing compound, a transition metal-containing compound, or a combination thereof.

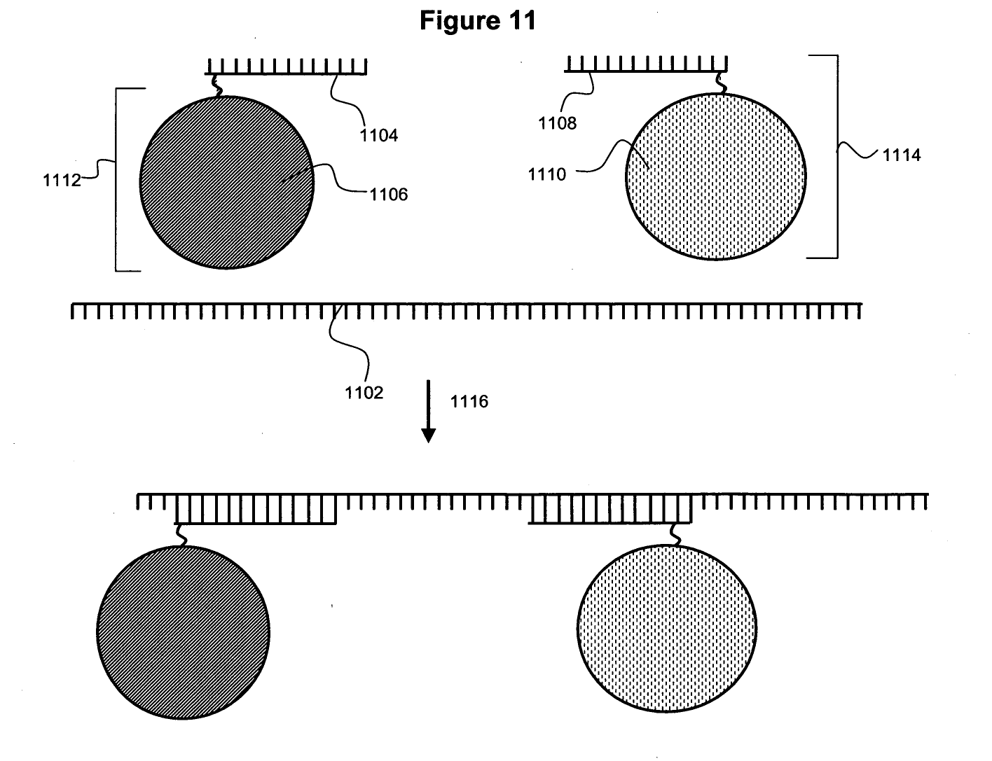

In FIG. 11, an embodiment for a use of two different RFID tags in the detection of nucleic acid in a sample is illustrated. A first RFID tag (1106) has a first capture oligo (1104) conjugated to or otherwise affixed to it to form a first RFID-oligo complex (1112). A second RFID tag (1110) has a second capture oligo (1108) conjugated to or otherwise affixed to it to form a second RFID-oligo complex (1114). The RFID-oligo complexes (1112 and 1114) are introduced to a sample suspected of containing a target nucleic acid (1102) (step 1116) under conditions to promote hybridization between the RFID-oligo complexes and the target nucleic acid (1102). Following this hybridization reaction, unbound RFID-oligo complexes are removed from the solution phase of the mixture, e.g., through binding to an immobilized binding partner comprising oligonucleotides complementary to capture oligo 1104 and capture oligo 1108, and the solution phase comprising the RFID-oligo complexes bound to the target nucleic acid is subjected to RFID interrogation (not shown). If the target nucleic acid is present, identification both RFID tags in close proximity to one another will be indicative of the presence of the target nucleic acid in the sample. In some instances, only one RFID complex will bind to a nucleic acid sequence from the sample, e.g., a non-target nucleic acid, and in such instances the interrogation will reveal only one of the RFID tags, indicating that the RFID tag is not associated with the target nucleic acid. In such instances where there is no target nucleic acid in the sample, most or all of the RFID-oligo complexes will be bound by the immobilized binding partners, thereby being removed from the solution to be interrogated. (Some of the RFID-oligo complexes may bind to a non-target nucleic acid from the sample, but will not be bound in close proximity to the other RFID-oligo complexes so will not be indicative of target nucleic acid, as described above.)

In an alternative embodiment, RFID-oligo complexes may be used in the detection of one or more nucleic acid sequences in a sample. In this embodiment, two or more RFID-oligo complexes are designed with capture oligos that hybridize to a target nucleic acid in close proximity to each other to form a pair. After hybridization, the sample is moved through an RFID detector where the presence of a particular nucleic acid sequence is determined by the spatially and temporally coincidental reading of an RFID-oligo complex pair (in a manner similar to that disclosed in the description for FIG. 11 above). In some embodiments, multiple pairs of RFID-oligo complexes may be used that bind to different portions of one target genome, or that bind to different genomes in a multiplexed assay. For example, an assay may include the use of RFID-oligo complex 1 and RFID-oligo complex 2 (to form a first RFID-oligo complex pair), which bind close to each other on a genomic sequence of one organism, and RFID-oligo complex 3 and RFID-oligo complex 4 (to form a second RFID complex pair), which bind close to each other on a genomic sequence of another organism. These four RFID-oligo complexes are then mixed with a sample suspected of containing the DNA of one or both of these organisms. If the DNA of the first organism is present, and the DNA of the second organism is not present in the sample, RFID-oligo complex 1 and RFID-oligo complex 2 will bind close to each on the genomic DNA of the first organism, and RFID-oligo 3 and RFID-oligo 4 will not bind. After hybridization has been permitted to occur, the reaction mix is then passed through an RFID reader, and the RFID identification tags of RFID-oligo complex 1 and RFID-oligo complex 2 will be read in close temporal proximity, and RFID-oligo complex 3 and RFID-oligo complex 4 will not be read in close temporal proximity. Detection of both signals of an RFID-oligo complex pair in close temporal proximity indicates the target nucleic acid was present in the sample. Detection of only one RFID-oligo complex signal over a given time frame, or the detection of multiple RFID-oligo complex signals over a given time frame not constituting an RFID-oligo complex pair indicates the target nucleic acid was not present in the sample. This embodiment includes the use of, in varied orders or combinations: (1) a first RFID-oligo complex comprising a first RFID tag and a first capture oligo complementary to a first region of a target nucleic acid, (2) a second RFID-oligo complex comprising a second RFID tag and a second capture oligo complementary to a second region of the target nucleic acid, wherein the first and second regions of the target nucleic acid are proximate to one another, and (3) a sample suspected of containing the target nucleic acid. The method includes mixing the first and second RFID-oligo complexes with the sample suspected of containing the target nucleic acid under conditions that will allow hybridization of the two RFID-oligo complexes to the target nucleic acid if there is perfect complementarity. The sample volume is then passed through an RFID reader. The coincidence of the reading of the two RFID tag signals in close temporal proximity indicates that the two RFID-oligo complexes are bound in close proximity to each other on the target nucleic acid, and hence the presence of the target nucleic acid in the sample can be detected. The detection of only a single RFID tag or the detection of RFID tag signals in close proximity to RFID tags that do not constitute a pair do not indicate that the target nucleic acid was present in the sample, and may be an indication that the target nucleic acid was absent from the sample.

Other embodiments of methods for genotyping one or more SNPs in a nucleic acid sample using RFID tags with associated capture oligos include the use of in varied orders or combinations: (1) an RFID tag with a capture oligo affixed or otherwise associated with it to form an RFID-oligo complex, (2) a sample containing target nucleic acid, and (3) a set of capture oligos for each SNP to be genotyped that is affixed or otherwise associated to a matrix, where the set comprises capture oligos complementary to the target nucleic acid and identical to one another except at the SNP position, where different subsets of the capture oligos contain different nucleotides at the SNP position, such that the set of capture oligos on the matrix contains at least one capture oligo for each possible nucleotide (e.g., A, T, G, or C) at the SNP position. For example, there may be four different subsets of capture oligos in a set: one comprised of capture oligos with an A at the SNP position, one comprised of capture oligos with a T at the SNP position, one comprised of capture oligos with a G at the SNP position, and one comprised of capture oligos with a C at the SNP position. In other embodiments, only two subsets of capture oligos may be used, one complementary to each of the known genotypes for the SNP position. In further embodiments, the complementary capture oligos for all subsets of capture oligos may also be immobilized at known locations on the matrix. The target nucleic acid may be fragmented such that it has a region extending beyond the portion that hybridizes to the capture oligos on the matrix. The capture oligo affixed or otherwise associated with the RFID-oligo complex has a sequence that is substantially complementary to a sequence of this overhanging region of the target nucleic acid. The method includes mixing the target nucleic acid with the capture oligos on the matrix under conditions that permit hybridization of target nucleic acid fragments to capture oligos perfectly complementary thereto. Where the SNP position of the capture oligos is not perfectly complementary to the target nucleic acid, hybridization does not typically occur. The reaction is washed or rinsed to remove any target nucleic acid that has not bound to its perfectly complementary capture oligo. The RFID-oligo complex is added to the reaction under conditions that permit binding of the capture oligo on the RFID-oligo complex to the overhang region of a target nucleic acid bound to a capture oligo on the matrix. The reaction is washed or rinsed to remove any unreacted RFID-oligo complexes (i.e., not bound to target nucleic acid). The matrix is scanned with an RFID reader and the RFID tags in the reacted RFID-oligo complexes are identified. In this manner, it is possible to correlate the detection of an RFID tag with a location at which an RFID-oligo complex is hybridized on the matrix to determine the genotype of the SNP based on the sequence of the capture oligo at that location on the matrix. In certain embodiments, multiple SNP positions can be genotyped simultaneously in this manner.

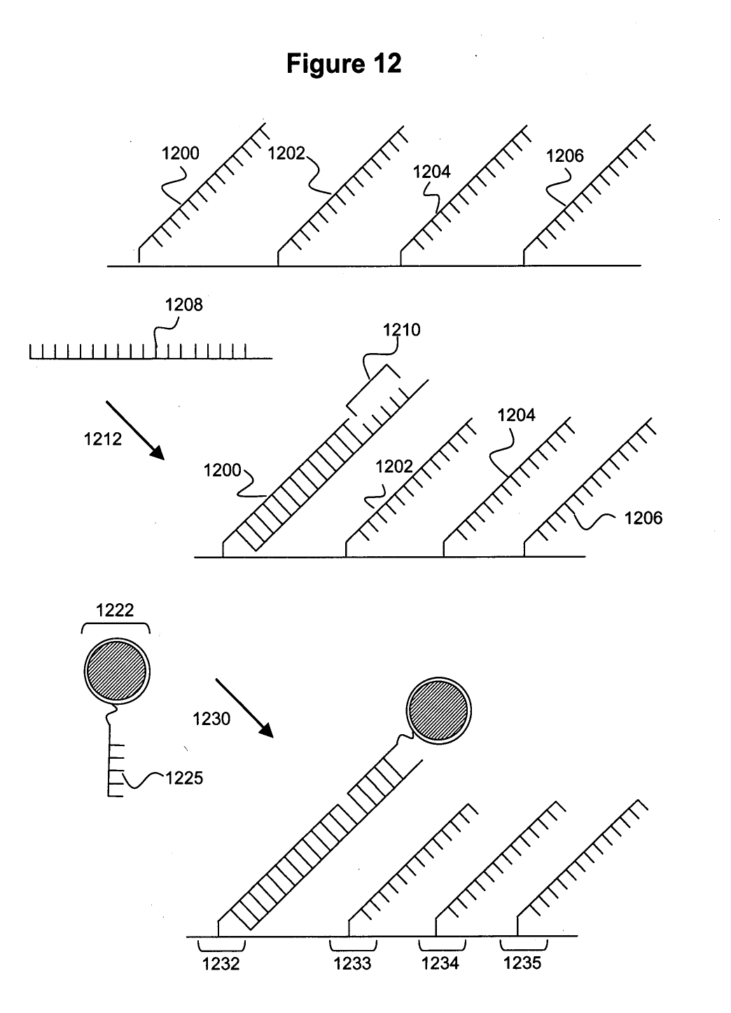

For example, in FIG. 12, a use of RFID tags for a detection of single nucleotide polymorphisms (SNPs) is illustrated. Capture oligos (1200, 1202, 1204, 1206) are designed for the detection of a SNP at a particular position and each have one of the four possible DNA nucleotides at the variable position, as described above. A sample suspected of containing target nucleic acid (1208) is introduced to the capture oligos (step 1212) under conditions to promote hybridization between the target nucleic acid and one of the capture oligos. The target nucleic acid preferentially binds to the capture oligo that has its complementary base pair at the variable position, as described above, to create immobilized target nucleic acid. In this figure, the first capture oligo (1200) binds to the target nucleic acid, and the other three capture oligos (1202, 1204 and 1206) do not hybridize to the target nucleic acid. The target nucleic acid has an overhanging region of DNA (1210) extending beyond the region hybridized with the oligonucleotide, which is complementary to another capture oligo (1225) of an RFID-oligo complex (1222). RFID-oligo complexes (1222) are added to the mixture (step 1230) under conditions that promote hybridization between the RFID-oligo complex (1222) and the immobilized target nucleic acid, thereby immobilizing the RFID-oligo complexes (1222) bound to the immobilized target nucleic acid. The RFID-oligo complexes that do not bind to the immobilized target nucleic acid (including those that may bind to other nucleic acids in the sample) are removed from the vessel, e.g., through a rinsing/washing step, and the vessel is subjected to RFID interrogation (not shown). Identification of a particular RFID tag is indicative of the presence of a particular RFID-oligo complex associated with the capture oligo at that location on the matrix (here, 1232), thus identifying the nucleotide at the variable position of the immobilized target nucleic acid and “genotyping” the SNP position in the sample.

In alternative embodiments, one or more SNPs may be detected in a nucleic acid sample without requiring the use of both target nucleic acid and capture oligos in RFID-oligo complexes. In one embodiment of this aspect, RFID tags associated with target nucleic acids are used to identify the presence of a SNP. This embodiment includes the use of, in varied orders or combinations: (1) an RFID tag with a portion of target nucleic acid from a sample affixed or otherwise associated with it to form an RFID-oligo complex and (2) a set of capture oligos for each SNP to be genotyped that is affixed or otherwise associated to a matrix, where the set comprises capture oligos complementary to the target nucleic acid and identical to one another except at the SNP position, where different subsets of the capture oligos contain different nucleotides at the SNP position, such that the set of capture oligos contains at least one capture oligo for each possible nucleotide (e.g., A, T, G, or C) at the SNP position, as described above. The method includes mixing the RFID-oligo complex with the capture oligos on the matrix under conditions that permit hybridization of target nucleic acid fragments to capture oligos perfectly complementary thereto. Where the SNP position of the capture oligo is not perfectly complementary to the target nucleic acid strand on the RFID-oligo complex, hybridization of the two nucleic acid strands typically does not occur. The reaction is then washed or rinsed to remove any target nucleic acid that has not bound to a perfectly complementary capture oligo. The matrix is scanned with an RFID reader and the RFID tags in the reacted RFID-oligo complexes are identified. In this manner, it is possible to correlate the detection of an RFID tag with a location at which an RFID-oligo complex is hybridized on the matrix to determine the genotype of a SNP based on the sequence of the capture oligo at that location on the matrix.

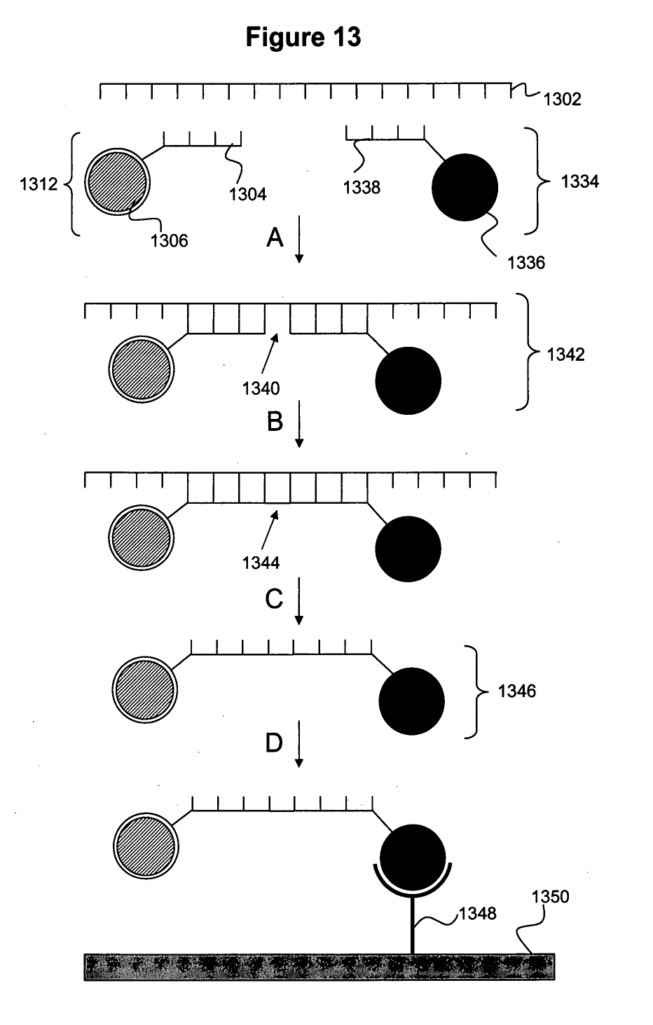

In some embodiments of the invention, RFID-oligo complexes are used in combination with oligo-reactive group complexes in the detection of one or more target nucleic acids in a sample. FIG. 13 illustrates one such embodiment in which an RFID-oligo complex (1312) comprises an RFID tag (1306) and a capture oligo (1304) conjugated to or otherwise affixed to it, and an oligo-reactive group complex (1334) comprises a reactive group (1336) and an oligonucleotide (1338) conjugated to or otherwise affixed to it. The oligos (1304 and 1338) of the RFID-oligo complex (1312) and the oligo-reactive group complex (1334) are designed to have sequences substantially complementary to adjacent regions of a target nucleic acid such that they will bind end-to-end (or substantially adjacent to each other) on the target nucleic acid. Double-stranded target nucleic acid can be denatured (not shown) to produce the single-stranded target nucleic acid (1302) shown. In step A, the RFID-oligo complex (1312) and the oligo-reactive group complex (1334) are contacted with a sample suspected of containing target nucleic acid (1302) under conditions to promote hybridization between both the RFID-oligo complex (1312) and the target nucleic acid (1302), and the oligo-reactive group complex (1334) and the target nucleic acid (1302). If the target nucleic acid is present in the sample, the hybridization of these entities (1312, 1334 and 1302) forms hybridized RFID complex (1342). As shown in this particular embodiment of hybridized RFID complex (1342), the oligos (1304 and 1338) of the RFID-oligo complex (1312) and the oligo-reactive group complex (1334) have hybridized end-to-end (adjacent to each other) on the target nucleic acid (1302). Also seen on hybridized RFID complex (1342) is nick junction (1340). Nick junction (1340) represents the lack of a phosphodiester bond between the annealed oligos (1304 and 1338) of the RFID-oligo complex (1312) and the oligo-reactive group complex (1334) on the target nucleic acid (1302). In step B, hybridized RFID complex (1342) is treated with a reagent capable of forming a linkage between annealed oligos (1304 and 1338) (e.g., DNA ligase, which catalyzes formation of a phosphodiester bond at nick junction (1340) to form ligated nick junction (1344)). In certain embodiments, (as shown in step C) the double-stranded product can be denatured (e.g., via high temperature, salt, or other conditions) to yield single-stranded target nucleic acid and single-stranded RFID-oligo-reactive group complex (1346). In certain embodiments, as shown in step D, RFID-oligo-reactive group complex (1346) can be contacted with, for example, a substrate (1350) with an immobilized binding partner (1348) affixed to or otherwise associated with the substrate. Immobilized binding partner (1348) is designed to preferentially bind to the reactive group (1336). The substrate is preferably washed or rinsed to remove any RFID-oligo complexes not in an RFID-oligo-reactive group complex (e.g., unreacted RFID-oligo complexes that did not bind to target nucleic acid and, therefore, were not ligated to oligo-reactive group complexes), as well as the single-stranded target nucleic acid (thereby ensuring that the target agent does not come into contact with the RFID detection device). The substrate can be subjected to RFID interrogation (not shown). If the target nucleic acid is present, the reacted RFID-oligo complexes (having been ligated to oligo-reactive group complexes to form RFID-oligo-reactive group complexes) will remain on the substrate, and are subject to interrogation. Identification of a particular RFID tag is indicative of the presence of the target nucleic acid in the sample. In such instances where there is no target nucleic acid in the sample, the foregoing RFID-oligo-reactive group complex does not form, and no RFID tags are retained for detection. Alternatively, if the specificity of binding of the two capture oligos to the target nucleic acid is sufficiently high, hybridized RFID complex (1342) need not be treated with a reagent capable of forming a linkage between annealed oligos (1304 and 1338), and therefore is not denatured, prior to being contacted with the substrate (1350) comprising immobilized binding partner (1348).

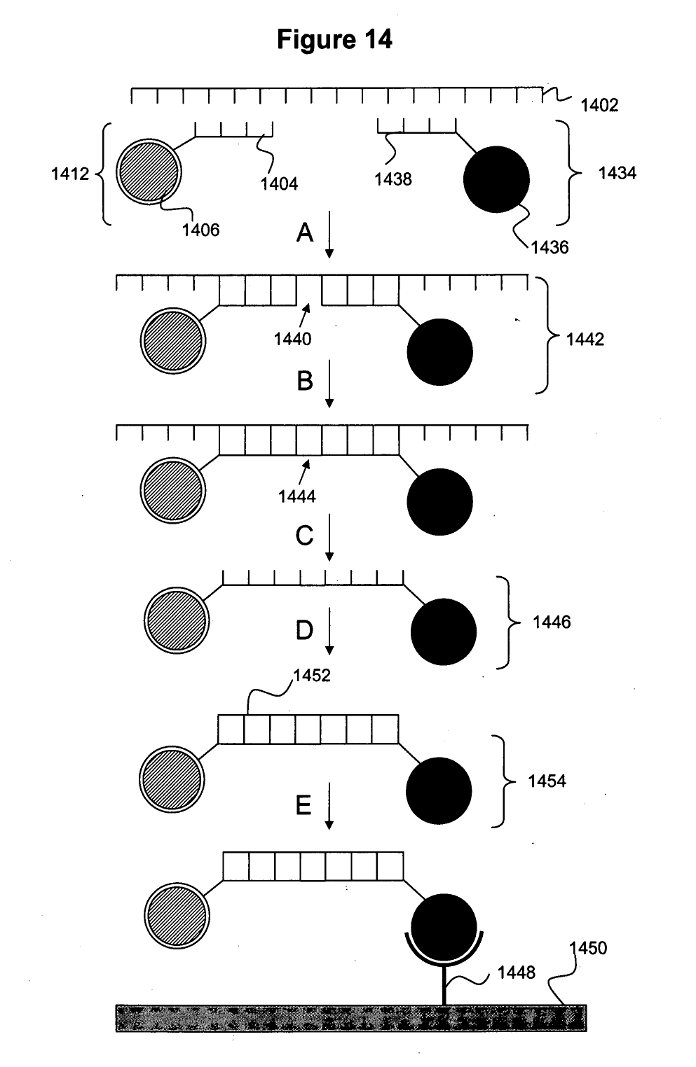

FIG. 14 illustrates an embodiment similar to that shown in FIG. 13. RFID-oligo complex (1412) comprises an RFID tag (1406) and an oligonucleotide (1404) conjugated to or otherwise affixed to it. An oligo-reactive group complex (1434) comprises a reactive group (1436) and an oligonucleotide (1438) conjugated to or otherwise affixed to it. The oligos (1404 and 1438) of the RFID-oligo complex and the oligo-reactive group complex are designed to have sequences substantially complementary to adjacent regions of a target nucleic acid such that they will bind end-to-end on the target nucleic acid. Double-stranded target nucleic acid can be denatured (not shown) to produce the single-stranded target nucleic acid shown (1402). In step A, the RFID-oligo complex (1412) and the oligo-reactive group complex (1434) are contacted with a sample suspected of containing target nucleic acid (1402) under conditions to promote hybridization between the RFID-oligo complex (1412), the oligo-reactive group complex (1434), and the target nucleic acid (1402). The hybridization of these entities (1412, 1434 and 1402) forms hybridized RFID complex (1442). As shown in this particular embodiment of hybridized RFID complex (1442), the oligos (1404 and 1438) of the RFID-oligo complex and the oligo-reactive group complex have hybridized end-to-end (adjacent to each other) on the target nucleic acid. Also seen on hybridized RFID complex (1442) is nick junction (1440). Nick junction (1440) represents the lack of a phosphodiester bond between the annealed oligos (1404 and 1438) of the RFID-oligo complex (1412) and the oligo-reactive group complex (1434) on the target nucleic acid (1402). In step B, hybridized RFID complex (1442) is treated with a reagent capable of forming a linkage between annealed oligos (1404 and 1438) (e.g., DNA ligase which catalyzes a strand joining reaction at nick junction (1440) to form ligated nick junction (1444)). In certain embodiments, as shown in step C, the product from step B can be denatured (e.g., via high temperature, salt, or other conditions) to separate the target nucleic acid strand from the now ligated oligos conjugated to the RFID and the reactive group to form the single-stranded RFID-oligo-reactive group complex (1446). In certain embodiments, as shown in step D, single-stranded oligonucleotide (1452) complementary to the oligo of single-stranded RFID-oligo-reactive group complex (1446) is added to the mixture under conditions to promote hybridization to form double-stranded RFID-oligo-reactive group complex (1454). The formation of a double-stranded complex assists in the stabilization of the complex. In certain embodiments, as illustrated in step E, double-stranded RFID-oligo-reactive group complex (1454) can be contacted with, for example, a substrate (1450) with an immobilized binding partner (1448) conjugated to or otherwise affixed to the substrate. Immobilized binding partner (1448) is designed to preferentially bind to the reactive group (1436) of double-stranded RFID-oligo-reactive group complex (1454). The substrate is preferably washed or rinsed to remove any RFID-oligo complexes that have not formed into a double-stranded RFID-oligo-reactive group complex (e.g., unreacted RFID-oligo complexes that did not bind to target nucleic acid and, therefore, were not ligated to oligo-reactive group complexes), as well as the single-stranded target nucleic acid (thereby ensuring that the target agent does not come into contact with the RFID detection device). The substrate can be subjected to RFID interrogation (not shown). If the target nucleic acid is present, the RFID-oligo complexes (having formed with the oligo-reactive group complexes into double-stranded RFID-oligo-reactive group complexes) will remain on the substrate, and are subject to interrogation. Identification of a particular RFID tag is indicative of the presence of the target nucleic acid in the sample. In such instances where there is no target nucleic acid in the sample, the foregoing double-stranded RFID-oligo-reactive group complex does not form, and no RFID tags are retained for detection.

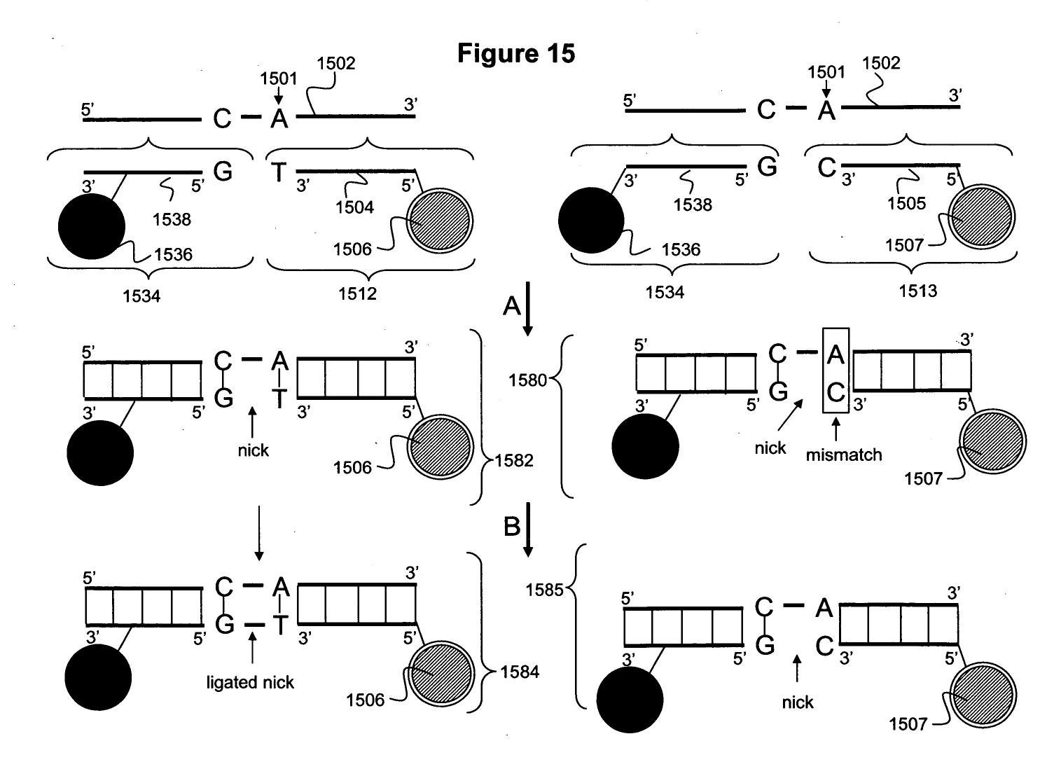

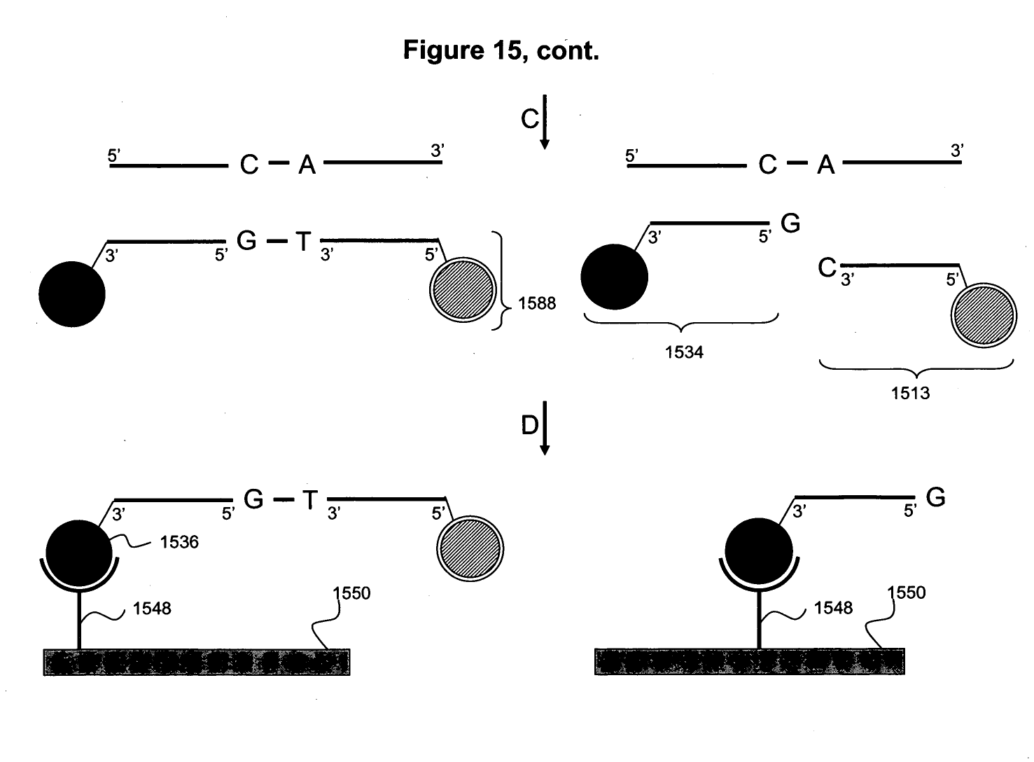

FIG. 15 illustrates another embodiment for genotyping SNPs in target nucleic acids. This method takes advantage of the fact that DNA ligase requires Watson-Crick base-pair complementarity at the nick junction to efficiently seal the nick. Similar to FIGS. 13 and 14, this use of RFID tags employs both RFID-oligo complexes and oligo-reactive group complexes that hybridize adjacent to each other on the target nucleic acid, as well as the use of DNA ligase to seal the nick between these adjacently-hybridized oligos. See, e.g., Landegren et al., Science 241:1077 (1988), and Cao, Trends in Biotechnology 22:38 (2004). Thus, DNA ligase is used to distinguish single nucleotide correct base-pairing from single nucleotide mismatch base-pairing between an RFID-oligo complex and a target nucleic acid. Double-stranded target nucleic acid can be denatured (not shown) to produce the single-stranded form shown (1502), which, in this example, has an “A” at the SNP position (1501). The individual components of the reaction are shown. The left column depicts a first RFID-oligo complex (1512) comprising a first RFID tag (1506) and a first capture oligo (1504) with a “T” at its 3′ end. Capture oligo (1504) is complementary to the target nucleic acid (1502), and its 3′ terminus aligns with the SNP position (1501) in the target nucleic acid when it is hybridized to the target nucleic acid. The right column depicts a second RFID-oligo complex (1513) comprising a second RFID tag (1507) and a second capture oligo (1505) identical to the first capture oligo (1504), but with a “C” at its 3′ position. The first and second RFID tags (1506 and 1507) can be distinguished by an RFID reader, e.g., using their individual identification numbers. In certain embodiments, RFID-oligo complexes representing all four nucleotide bases at the 3′ position may be used (e.g., if the possible SNP genotypes are unknown), though typically, the genotype of any one SNP position is one of two alternate bases. The target nucleic acid (1502) is shown in this particular embodiment with the middle bases of “C” and “A”, with the “A” position being the SNP position (1501). Oligo-reactive group complex (1534) is comprises a reactive group (1536) and a third capture oligo (1538) with a “G” at the 5′ position, which is complementary to the target nucleic acid in a portion adjacent to and 5′ of the SNP position (1501).

In step A, the individual components are mixed or otherwise contacted with each other under conditions to promote hybridization of the oligo-reactive group complexes and RFID-oligo complexes to the target nucleic acid to produce a “basepair match RFID complex” (1582) and a “basepair mismatch RFID complex” (1580). Basepair match RFID complex (1582) has a nick between the capture oligos of the oligo-reactive group complex and the first RFID-oligo complex (1512) as well as complementarity (i.e., a basepair match) between the “T” on the 3′ end of the capture oligo of the first RFID-oligo complex (1512) and the “A” of the target nucleic acid (1502) at the SNP position (1501). Basepair mismatch RFID complex (1580) has a nick between the capture oligos of the oligo-reactive group complex and the second RFID-oligo complex (1513) as well as no complementarity (i.e., a basepair mismatch) between the “C” on the 3′ end of the capture oligo of the second RFID-oligo complex (1513) and the “A” of the target nucleic acid (1502) at the SNP position (1501). In step B, a reagent capable of forming a linkage between annealed capture oligos (e.g., DNA ligase) is added to the reaction mix and ligates the nicks in products where there is complementarity between the 3′ end of a capture oligo of an RFID-oligo complex and the corresponding position on the target nucleic acid (in this case, the ligase will join capture oligos 1538 and 1504, but not capture oligos 1538 and 1505). The result of the addition of ligase is ligated RFID super-complex (1584) and non-ligated RFID super-complex (1585). Ligated RFID super-complex (1584) has complementarity (i.e., a basepair match) between the 3′ position of the capture oligo on the first RFID-oligo complex (1512) and the SNP position (1501) on the target nucleic acid (1502), and therefore the DNA ligase is able to create a phosphodiester bond between the capture oligos of the oligo-reactive group complex (1534) and the first RFID-oligo complex (1512). Non-ligated RFID super-complex (1585) does not have complementarity (i.e., has a basepair mismatch) between the 3′ position of the capture oligo of the second RFID-oligo complex (1513) and the SNP position (1501) on the target nucleic acid (1502), and therefore DNA ligase is not typically able to create a phosphodiester bond between the capture oligos of the oligo-reactive group complex (1534) and the second RFID-oligo complex (1513). In certain embodiments, as shown in step C, the double-stranded products of step B can be denatured to yield single-stranded RFID super-complex (1588), single-stranded target nucleic acid, oligo-reactive group complex (1534) and second RFID-oligo complex (1513). Single-stranded RFID super-complex (1588) represents the oligo-reactive group complex ligated to the first RFID-oligo complex from the ligated RFID super-complex (1584). In certain embodiments, as illustrated in step D, the reaction mix, including single-stranded RFID super-complex (1588), can be contacted with, for example, a substrate (1550) with an immobilized binding partner (1548) bound to the substrate. The immobilized binding partner (1548) is designed to preferentially bind to reactive group (1536), and thereby can immobilize the single-stranded RFID super-complex (1588). The substrate is preferably washed or rinsed to remove any unreacted RFID-oligo complexes (e.g., RFID-oligo complexes that have not been ligated to oligo-reactive group complexes to form a single-stranded RFID super-complexes (1588)), as well as the single-stranded target nucleic acid (thereby ensuring that the target agent does not come into contact with the RFID detection device). If the target nucleic acid is present, the RFID-oligo complexes that have ligated to the oligo-reactive group complexes to form single-stranded RFID super-complexes (1588) will remain on the substrate, and are subject to interrogation. Identification of a particular RFID tag will be indicative of the genotype of the SNP in the target nucleic acid in the sample. In such instances where there is no target nucleic acid in the sample, the foregoing single-stranded RFID complexes do not form, and no RFID tags are retained for detection. For example, the first RFID tag (1506) is the only one retained on the substrate in FIG. 15, indicating that the genotype of the source of the target nucleic acid at the SNP position is “A” (or “AA,” in the case of a diploid organism). If the genotype of the source of the target nucleic acid were “G” (or “GG,” in the case of a diploid organism), only the second RFID tag (1507) would have been retained and detected on the substrate. If the genotype of a diploid source of the target nucleic acid were heterozygous (“AG”), both RFID tags (1506 and 1507) would have been retained and detected on the substrate.

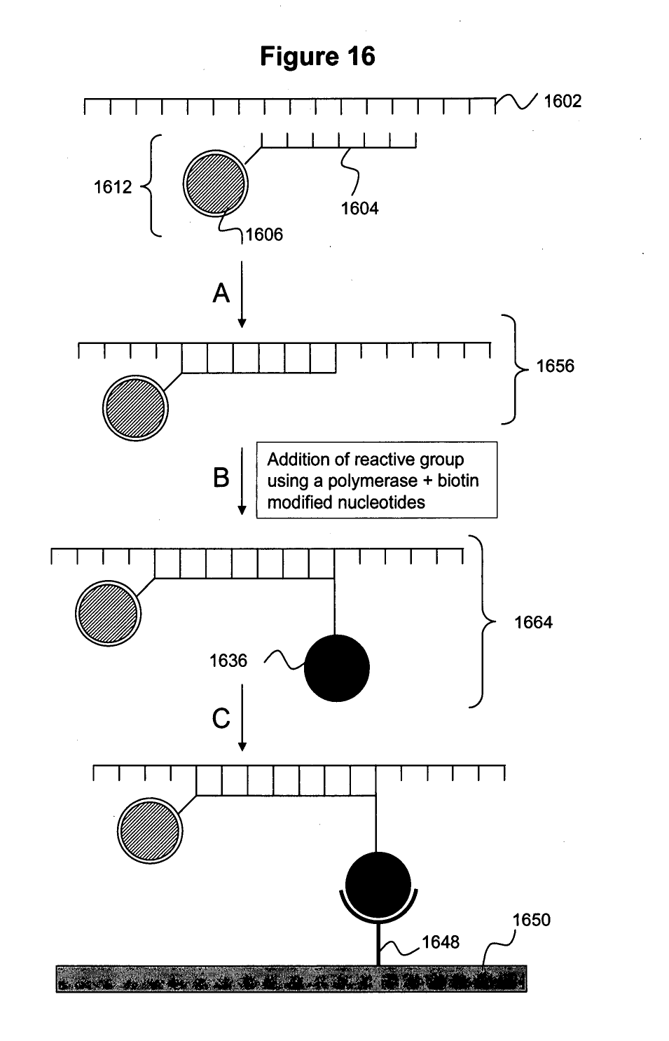

FIG. 16 illustrates a further embodiment of the invention in which RFID-oligo complexes are used in combination with reactive groups in the detection of one or more target nucleic acids in a sample. An RFID-oligo complex (1612) comprises an RFID tag (1606) and a capture oligo (1604) conjugated to or otherwise affixed to it. The capture oligo (1604) of the RFID-oligo complex (1612) is designed to have a sequence complementary to a target nucleic acid. Double-stranded target nucleic acid can be denatured (not shown) to produce the single-stranded form shown (1602). In step A, the RFID-oligo complex (1612) is contacted with a sample suspected of containing target nucleic acid (1602) under conditions to promote hybridization between the RFID-oligo complex (1612) and the target nucleic acid (1602) to form a hybridized RFID complex (1656). Such conditions may be stringent conditions to reduce or eliminate noncomplementary binding between the RFID-oligo complex and any nontarget DNA in the sample. In step B, a reactive group (1636) is conjugated or otherwise affixed to the hybridized RFID complex to form an RFID super-complex (1664). Specifically, in this example, a polymerase (e.g., E. coli polymerase I, Taq polymerase, etc.) and biotinylated nucleotides (along with appropriate reaction buffers and components) are added to the reaction mix in order to extend the capture oligo (1604) hybridized to the target nucleic acid (1602), thereby extending the double-stranded region and adding a reactive group (1636) to the hybridized RFID complex to form the RFID super-complex (1664). In certain embodiments, only a single modified nucleotide is added that is complementary to the nucleotide position immediately adjacent to the end of the capture oligo, further adding specificity to the method, since it is unlikely that the polymerase will have the required template to allow addition of the modified nucleotide at a nonspecific binding event between the RFID-oligo complex and nontarget DNA. In further embodiments, the region of double-stranded region is further elongated by adding nonmodified nucleotides in addition to the modified nucleotides. For example, if the target DNA contains the sequence “GAT” in the region immediately adjacent to that bound by the capture oligo, a practitioner could add unmodified C and T, and modified A nucleotides, which would extend the double-stranded region by three nucleotides.

In certain embodiments, as illustrated in step C of FIG. 16, the RFID super-complex (1664) can be contacted with, for example, a substrate (1650) with an immobilized binding partner (1648) bound to the substrate. The immobilized binding partner (1648) is designed to preferentially bind to the reactive group (1636) of the RFID super-complex (1664). The substrate is preferably washed or rinsed to remove any RFID-oligo complexes that have not hybridized to target nucleic acid and associated with the reactive group to form an RFID super-complex. The substrate can be subjected to RFID interrogation (not shown). If the target DNA nucleic acid is present, the RFID-oligo complexes (associated with target nucleic acid and reactive group to form RFID super-complexes) will remain on the substrate. Identification of a particular RFID tag is indicative of the presence of the target nucleic acid in the sample. In such instances where there is no target nucleic acid in the sample, the foregoing RFID super-complexes do not form, and no RFID tags are retained for detection.

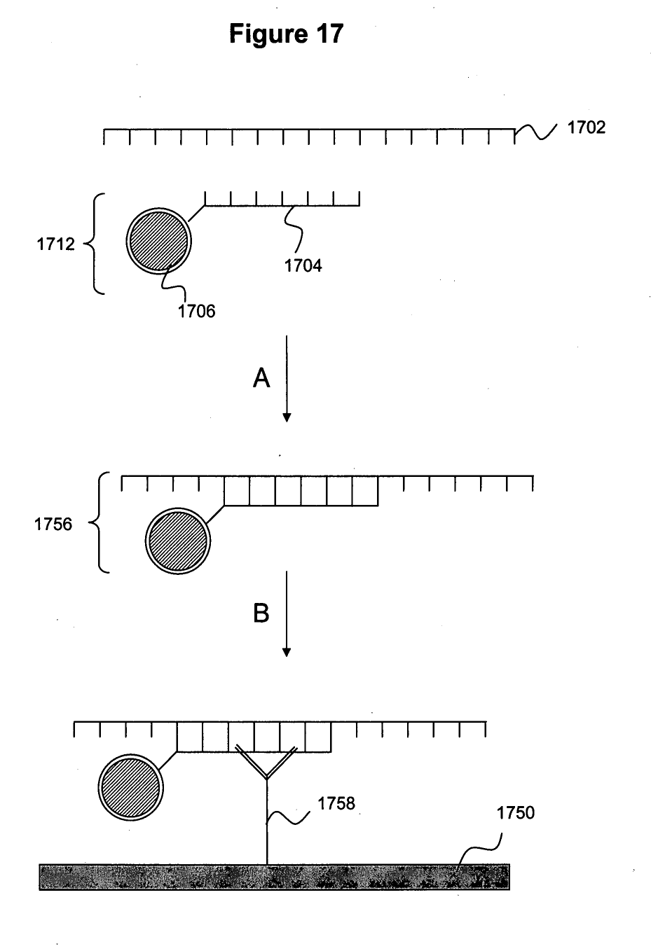

FIG. 17 illustrates another embodiment of a method for using RFID tags in the detection of nucleic acid in a sample. In this embodiment, DNA:RNA hybrids associated with RFID tags are immobilized and the RFID tags are subsequently detected. An RFID tag (1706) has an RNA oligonucleotide (1704) conjugated to or otherwise affixed to it to form an RFID-oligo complex (1712). The RNA oligo (1704) of the RFID-oligo complex is designed to have a sequence complementary to a target DNA. Double-stranded target DNA can be denatured (not shown) to produce the single-stranded target DNA shown (1702). In step A, the RFID-oligo complex (1712) is contacted with a sample suspected of containing target DNA (1702) under conditions to promote hybridization between the RFID-oligo complex and the target DNA (1702). Such conditions may be stringent conditions to reduce or eliminate noncomplementary binding between the RFID-oligo complex and any nontarget DNA in the sample. The hybridization of these entities (1712 and 1702) forms hybridized RFID complex (1756), which comprises a DNA:RNA hybrid where the RNA oligo is annealed to the target DNA. In certain embodiments, as illustrated in step B, hybridized RFID complex (1756) can be contacted with, for example, a substrate (1750) with an anti-DNA:RNA antibody (1758) bound to the substrate. The anti-DNA:RNA antibody (1758) is designed to specifically bind to a DNA:RNA hybrid, and not bind to single-stranded DNA, single-stranded RNA, double-stranded DNA, or double-stranded RNA. In this example, the anti-DNA:RNA antibody (1758) specifically binds to the DNA:RNA hybrid of hybridized RFID complex (1756). The substrate is preferably washed or rinsed to remove any RFID-oligo complexes that have not bound to target DNA to form a DNA:RNA hybrid (unreacted RFID-oligo complexes). The substrate can be subjected to RFID interrogation (not shown). If the target DNA is present, the reacted RFID-oligo complexes (having bound to the target DNA to form into hybridized RFID complexes) remain on the substrate and are subject to interrogation. Identification of a particular RFID tag is indicative of the presence of the target nucleic acid in the sample. In such instances where there is no target nucleic acid in the sample, the foregoing double-stranded RFID complexes do not form, and no RFID tags are retained for detection. Note that in other alternatives to this embodiment, the target nucleic acid (1702) could be RNA and the oligonucleotide (1704) could be DNA.

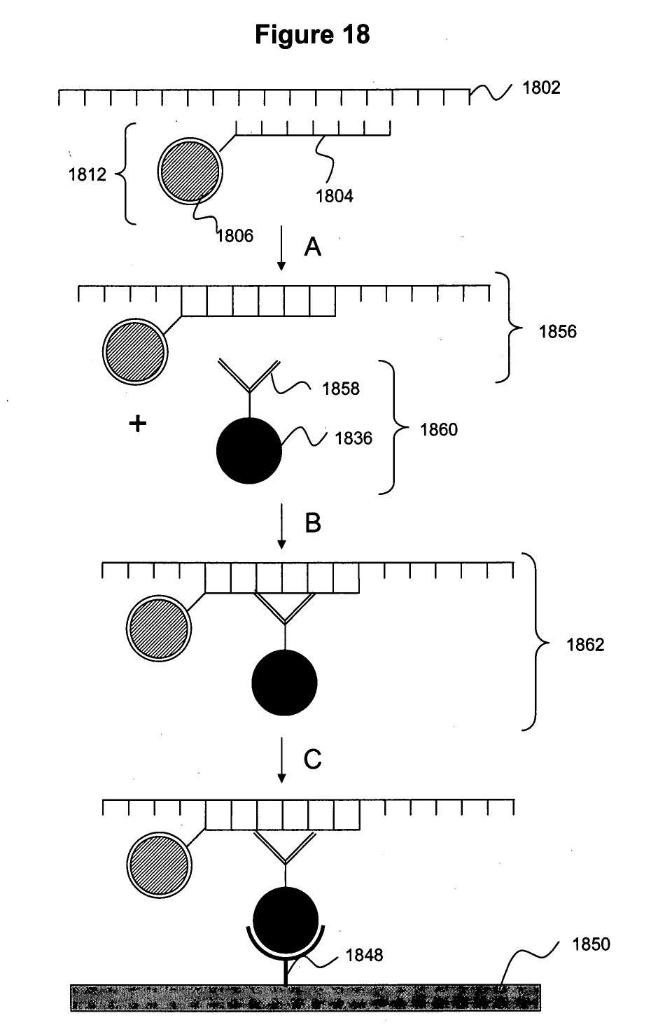

In certain embodiments, RFID complexes may be used in combination with antibody-reactive group complexes in the detection of one or more nucleic acid sequences (target agents) in a sample. For example, FIG. 18 illustrates an embodiment similar to that shown in FIG. 17 that features the use of an antibody-reactive group complex comprising a reactive group conjugated to an anti-DNA:RNA antibody, where the reactive group specifically binds to an immobilized binding partner on a substrate, thereby immobilizing an RFID-target DNA complex on the substrate. This embodiment includes the use of, in varied orders or combinations: (1) an RFID-oligo complex (1812) comprising an RFID tag (1806) conjugated or otherwise affixed to an RNA oligo (1804), (2) an antibody-reactive group complex (1860) comprising a reactive group (1836) and an antibody (1858) specific for DNA:RNA hybrids, (3) an immobilized binding partner (1848) affixed to or otherwise associated with a substrate (1850), and (4) a sample suspected of containing a target nucleic acid. In certain embodiments, the oligo (1804) of the RFID-oligo complex is RNA and is designed to have a sequence complementary to at least a portion of a target DNA such that it will preferentially bind to the target DNA if the latter is present. Double-stranded target DNA can be denatured (not shown) to produce the single-stranded form shown (1802). In step A, the RFID-oligo complex (1812) is contacted with a sample suspected of containing target DNA (1802) under conditions to promote hybridization between the RFID-oligo complex (1812) and the target DNA (1802). Such conditions may be stringent conditions to reduce or eliminate noncomplementary binding between the RFID-oligo complex and any nontarget DNA in the sample. The hybridization of these entities (1812 and 1802) forms hybridized RFID-complex (1856), which comprises a DNA:RNA hybrid where the RNA oligo annealed to the target DNA. In step B, antibody-reactive group complex (1860) comprising a reactive group (1836) and an anti-DNA:RNA antibody (1858) is introduced to the hybridized RFID-oligo complexes. Anti-DNA:RNA antibody (1858) is designed to preferentially bind to the DNA:RNA hybrid portion of the hybridized RFID-oligo complex (1856) to form RFID super-complex (1862). In certain embodiments, as illustrated in step C, RFID super-complex (1862) can be contacted with, for example, a substrate (1850) with an immobilized binding partner (1848) bound to the substrate. The immobilized binding partner (1848) is designed to preferentially bind to the reactive group (1836) of the RFID super-complex (1862), and this binding serves to immobilize the hybridized RFID-oligo complex on the substrate, thereby bringing the RFID tag of the RFID super-complex in close proximity to an RFID reader. RFID tags that are not in RFID super-complexes (not shown) may not be in close proximity to the RFID reader, and therefore may not be subjected to RFID interrogation. The substrate is optionally washed or rinsed to remove any unreacted RFID-oligo complexes (e.g., RFID-oligo complexes that did not hybridize to a target DNA and antibody-reactive group complex to form an RFID super-complex) from the reaction mix prior to RFID interrogation. The substrate can be subjected to RFID interrogation (not shown). If the target DNA is present, the reacted RFID-oligo complexes having bound target DNA and antibody-reactive group complex to form RFID super-complexes remain on the substrate and are subject to interrogation. Identification of a particular RFID tag is indicative of the presence of the target DNA in the sample. In such instances where there is no target DNA in the sample, the foregoing RFID super-complexes do not form, and no RFID tags are retained for detection. Note that in alternatives to this embodiment, the RFID tag (1806) could be conjugated to or otherwise associated with the anti-DNA:RNA antibody (1858) and the reactive group (1836) could be conjugated to or otherwise associated with the RNA oligonucleotide (1804). Note that in other alternatives to this embodiment, the target nucleic acid (1802) could be RNA and the oligonucleotide (1804) could be DNA.

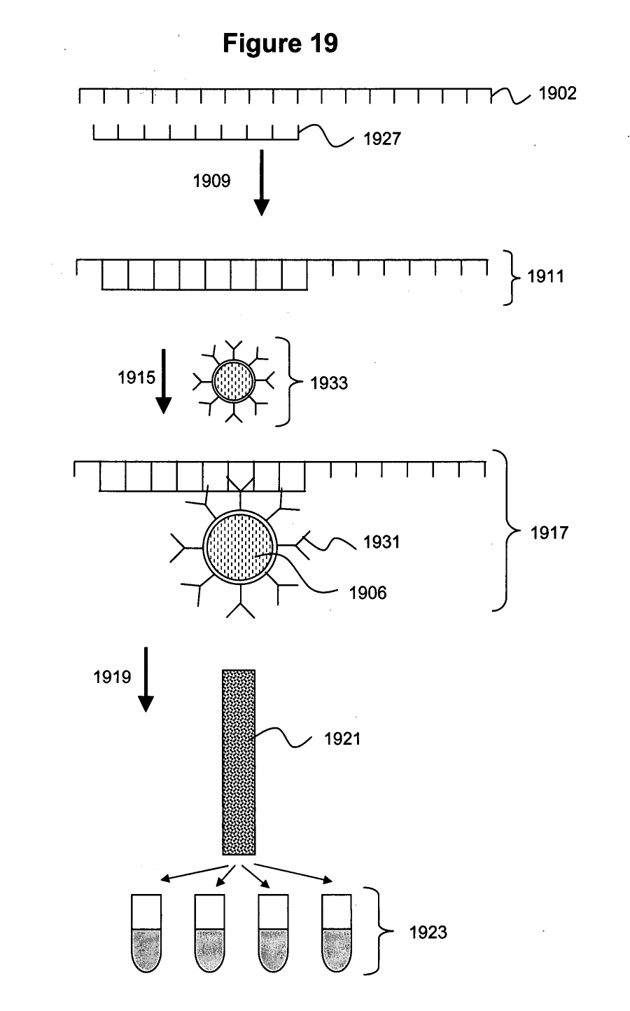

FIG. 19 illustrates a use of RFID tags in the detection of target nucleic acid in a sample using a size exclusion column to separate RFID tags associated with target nucleic acid from RFID tags not associated with target nucleic acid. Double-stranded target DNA can be denatured (not shown) to produce the single-stranded form shown (1902). In step 1909, target DNA (1902) and an RNA oligonucleotide (1927) are mixed under conditions to promote hybridization between the two entities. RNA oligonucleotide (1927) is designed to have a sequence substantially complementary to at least a portion of target DNA (1902), and will bind to target DNA if the latter is present. The result of this hybridization is a nucleic acid molecule (1911) comprising a double-stranded DNA:RNA hybrid region. In step 1915, RFID-antibody complex (1933) is contacted with the reaction mix. RFID-antibody complex (1933) comprises an anti-DNA:RNA antibody (1931) and an RFID tag (1906). The antibody (1931) of the RFID-antibody complex (1933) will bind to the DNA:RNA hybrid region of nucleic acid molecule (1911) to form RFID super-complex (1917). In certain embodiments, as illustrated in step 1919, the reaction mix is passed through a size-exclusion column (1921). In this manner, smaller species, such as unreacted RFID-antibody complexes (i.e., RFID-antibody complexes that are not associated with the double-stranded DNA:RNA hybrid region of nucleic acid molecule (1911)) and unbound RNA oligonucleotide (1927) will pass through the column faster than larger species. After the smaller species have passed through the column, the largest species, the RFID super-complexes, may be collected and subjected to RFID interrogation (not shown). Identification of a particular RFID tag is indicative of the presence of the target DNA in the sample. In such instances where there is no target DNA in the sample, the foregoing RFID super-complexes do not form, and no RFID tags are retained for detection. Note that in certain alternative embodiments, the single-stranded target nucleic acid (1902) is RNA and the oligonucleotide (1927) is DNA.

In alternative embodiments, a different type of column chromatography may be used, and these methods are well known to those of ordinary skill in the art. For example, an ion-exchange or affinity chromatography column may be used. The affinity chromatography column may have immobilized anti-DNA:RNA antibodies on the column. As the reaction sample comprising the RFID super-complex (1917) is passed through the column, the DNA:RNA hybrid region of nucleic acid molecule (1911) binds to the column, thereby immobilizing RFID super-complex (1917) on the column. Unreacted RFID-antibody complexes will not bind to the column and will pass through.

A solution may be subsequently added to the column to release the bound RFID super-complexes (1917) from the column. In this manner, it is possible to separate reacted from unreacted RFID-antibody complexes. The eluant tube containing only RFID super-complexes (comprising reacted antibody-RFID complexes) is subjected to RFID interrogation (not shown) and identification of a particular RFID tag is indicative of the presence of the target DNA in the sample. In such instances where there is no target DNA in the sample, the foregoing RFID super-complexes do not form, and no RFID tags are retained for detection.

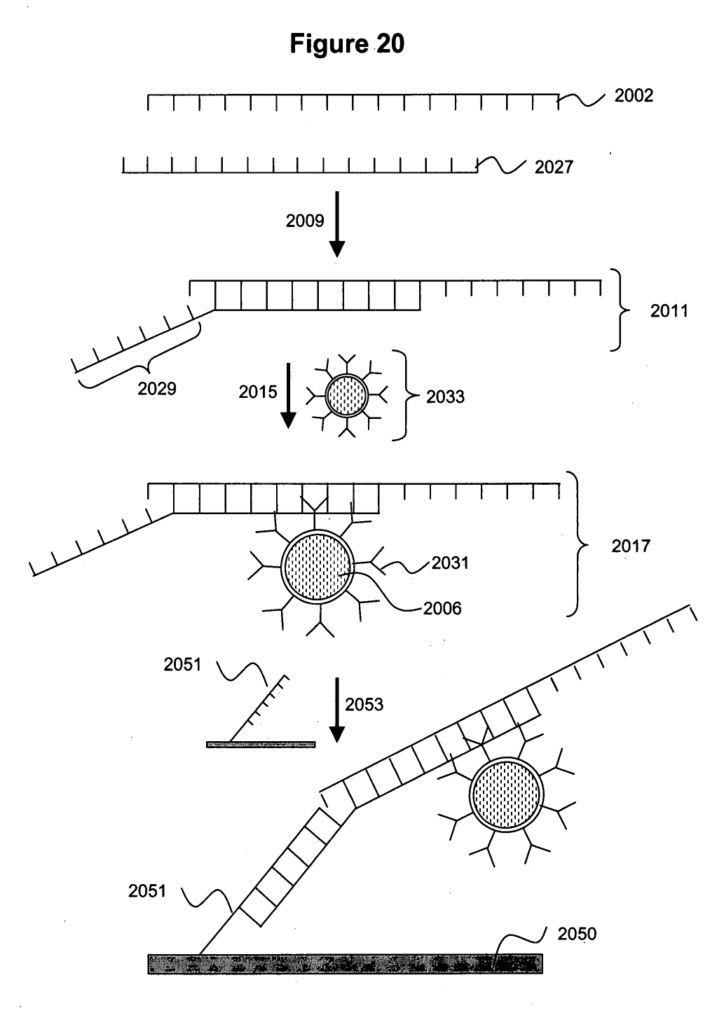

FIG. 20 provides an embodiment similar to that shown in FIG. 19, but in which an overhanging portion of an oligonucleotide is used to separate RFID tags associated with target nucleic acid from RFID tags not associated with target nucleic acid. Double-stranded target DNA can be denatured (not shown) to produce the single-stranded form shown (2002). In step 2009, target DNA (2002) and an RNA oligonucleotide (2027) are mixed under conditions to promote hybridization between the two entities. RNA oligonucleotide (2027) is designed to have a sequence complementary to target DNA (2002), and will bind to target DNA if the latter is present. In this figure, RNA oligonucleotide (2027), when hybridized to target DNA (2002), will have an overhanging portion (2029) that does not hybridize to the target DNA. The result of this hybridization is a nucleic acid molecule (2011) comprising a double-stranded DNA:RNA hybrid region and a single-stranded region comprising the overhanging portion (2029) of the RNA oligonucleotide (2027). In step 2015, RFID-antibody complex (2033) is contacted with the reaction mix. RFID-antibody complex (2033) comprises an anti-DNA:RNA antibody (2031) and an RFID tag (2006) conjugated or otherwise associated therewith. The antibody (2031) of the RFID-antibody complex (2033) will bind to the DNA:RNA hybrid region of the nucleic acid molecule (2011) to form an RFID super-complex (2017). In certain embodiments, as illustrated in step 2053, the reaction mix can be contacted with, for example, a substrate (2050) with an immobilized nucleic acid (2051) that is substantially complementary to the overhanging portion (2029) of RNA oligonucleotide (2027) under conditions that promote binding between the immobilized nucleic acid (2051) and the overhanging portion (2029). The substrate is preferably washed or rinsed to remove any unreacted RFID-antibody complexes (i.e., those that have not bound to the DNA:RNA hybrid to form an RFID super-complex). The substrate can be subjected to RFID interrogation (not shown). If the target DNA is present, the reacted RFID-antibody complexes (those bound to the DNA:RNA hybrid to form an RFID super-complex) remain on the substrate, and are subject to interrogation. Identification of a particular RFID tag is indicative of the presence of the target DNA in the sample. In such instances where there is no target DNA in the sample, the foregoing RFID super-complexes do not form, and no RFID tags are retained for detection. Note that in certain alternative embodiments, the single-stranded target nucleic acid (2002) could be RNA and the oligonucleotide (2027) could be DNA.

In embodiments related to that depicted in FIG. 20 an additional step is performed to separate unhybridized target DNA (2002) and RNA oligonucleotide (2027) from hybridized nucleic acid molecule (2011), either prior to or subsequent to addition of the RFID-antibody complex (2033). For example, the reaction mix can be contacted with a mix of immobilized binding partners comprising immobilized binding partners complementary to target DNA (2002) in the region complementary to RNA oligonucleotide (2027), and immobilized binding partners complementary to RNA oligonucleotide (2027) in the region complementary to target DNA (2002). Any unhybridized target DNA (2002) and RNA oligonucleotide (2027) will bind to the immobilized binding partners, but hybridized nucleic acid molecule (2011) will not. The RFID-antibody complex (2033) can be added to the solution phase comprising the unimmobilized nucleic acid molecule (2011) (if it was not previously added), and the solution phase can be subjected to RFID interrogation. Alternatively, the solution phase can be contacted with, for example, a substrate (2050) with an immobilized nucleic acid (2051) that is substantially complementary to the overhanging portion (2029) of RNA oligonucleotide (2027) under conditions that promote binding between the immobilized nucleic acid (2051) and the overhanging portion (2029). The substrate is preferably washed or rinsed to remove any unreacted RFID-antibody complexes (i.e., those that have not bound to the DNA:RNA hybrid to form an RFID super-complex) prior to RFID interrogation.

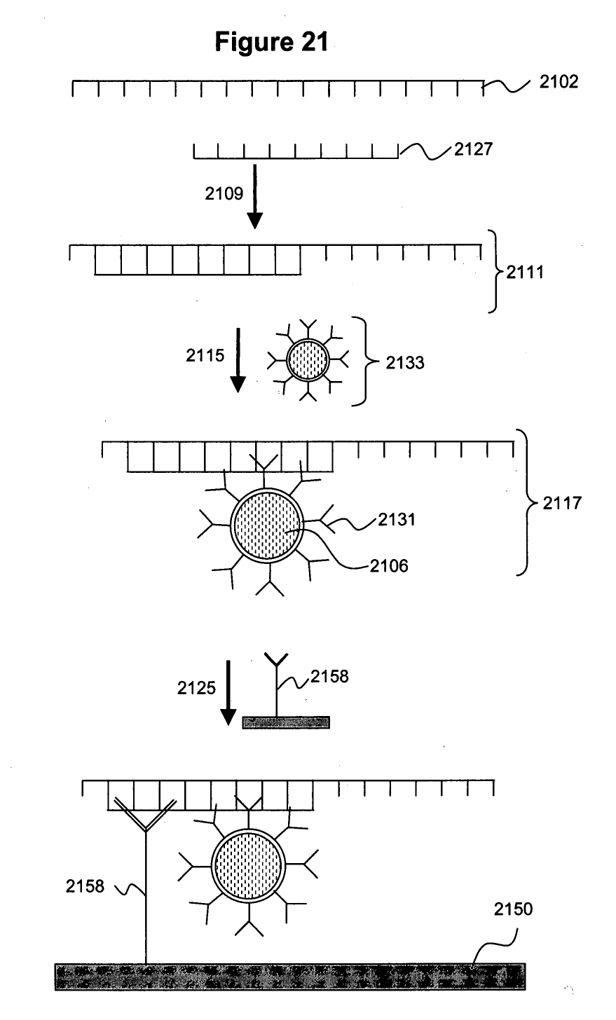

FIG. 21 illustrates another alternative embodiment that uses a secondary antibody to separate RFID tags associated with target nucleic acid from RFID tags not associated with target nucleic acid. Double-stranded target DNA can be denatured (not shown) to produce the single-stranded form shown (2102). In step 2109, target DNA (2102) and an RNA oligonucleotide (2127) are mixed under conditions to promote hybridization between the two entities. RNA oligonucleotide (2127) is designed to have a sequence substantially complementary to target DNA (2102), and will bind to target DNA if the latter is present. The result of this hybridization is a nucleic acid molecule (2111) comprising a double-stranded DNA:RNA hybrid region. In step 2115, RFID-antibody complex (2133) is contacted with the reaction mix. RFID-antibody complex (2133) comprises an anti-DNA:RNA antibody (2131) and an RFID tag (2106). The antibody (2131) of the RFID-antibody complex (2133) will bind to the DNA:RNA hybrid region of the nucleic acid molecule (2111) to form an RFID super-complex (2117). In certain embodiments, as illustrated in step 2125, the reaction mix can be contacted with, for example, a substrate (2150) with an immobilized anti-DNA:RNA antibody (2158) that recognizes the DNA:RNA hybrid. The substrate is preferably washed or rinsed to remove any RFID-antibody complexes that have not bound to the DNA:RNA hybrid to form an RFID super-complex. The substrate can be subjected to RFID interrogation (not shown). If the target DNA is present, the RFID-antibody complexes that bound DNA:RNA hybrids to form RFID super-complexes remain on the substrate, and are subject to interrogation. Identification of a particular RFID tag is indicative of the presence of the target DNA in the sample. In such instances where there is no target DNA in the sample, the foregoing RFID super-complexes do not form, and no RFID tags are retained for detection. The anti-DNA:RNA antibodies (2131) of the RFID-antibody complex (2133) and the anti-DNA:RNA antibodies (2158) on the substrate (2150) may be the same antibodies or may be different. The antibodies (2158) on the substrate (2150) also may, in certain embodiments, be an antibodies against the anti-DNA:RNA antibodies (2131) of the RFID-antibody complex (2133). In addition, in alternative embodiments, the nucleic acid molecule (2111) is bound to the antibodies on the substrate (2150) prior to being exposed to the RFID-antibody complex (2133). The RFID-antibody complex (2133) that does not bind to the nucleic acid molecule (2111) bound to the substrate (2150) is removed (e.g., by washing) prior to detection. Note that in certain alternative embodiments, the single-stranded target nucleic acid (2102) could be RNA and the oligonucleotide (2127) could be DNA.

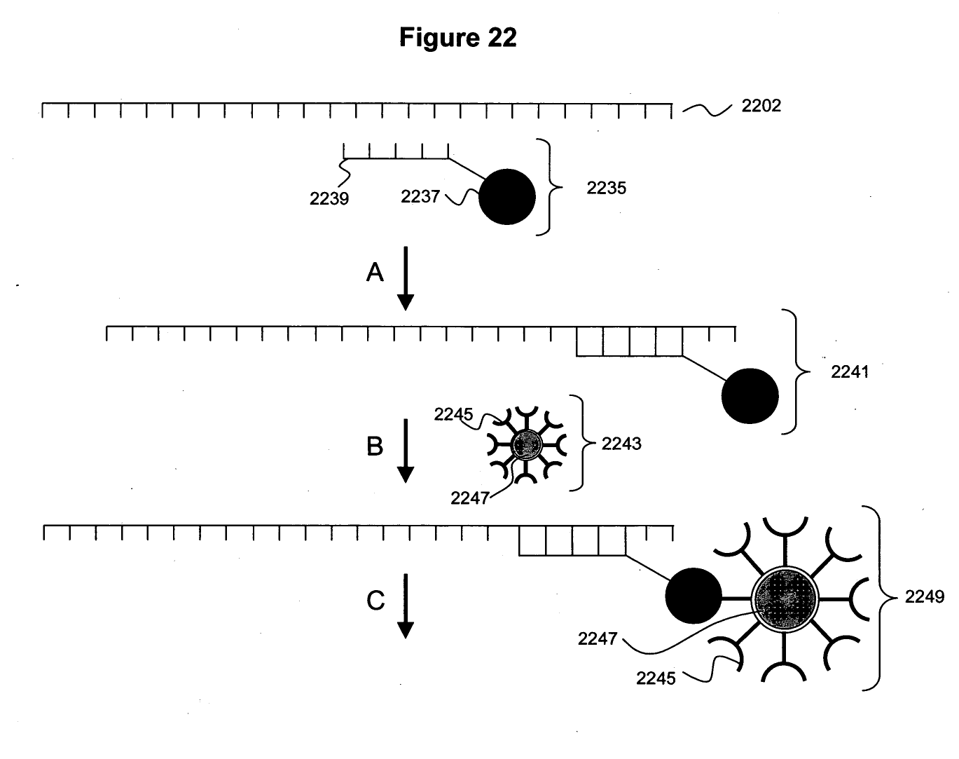

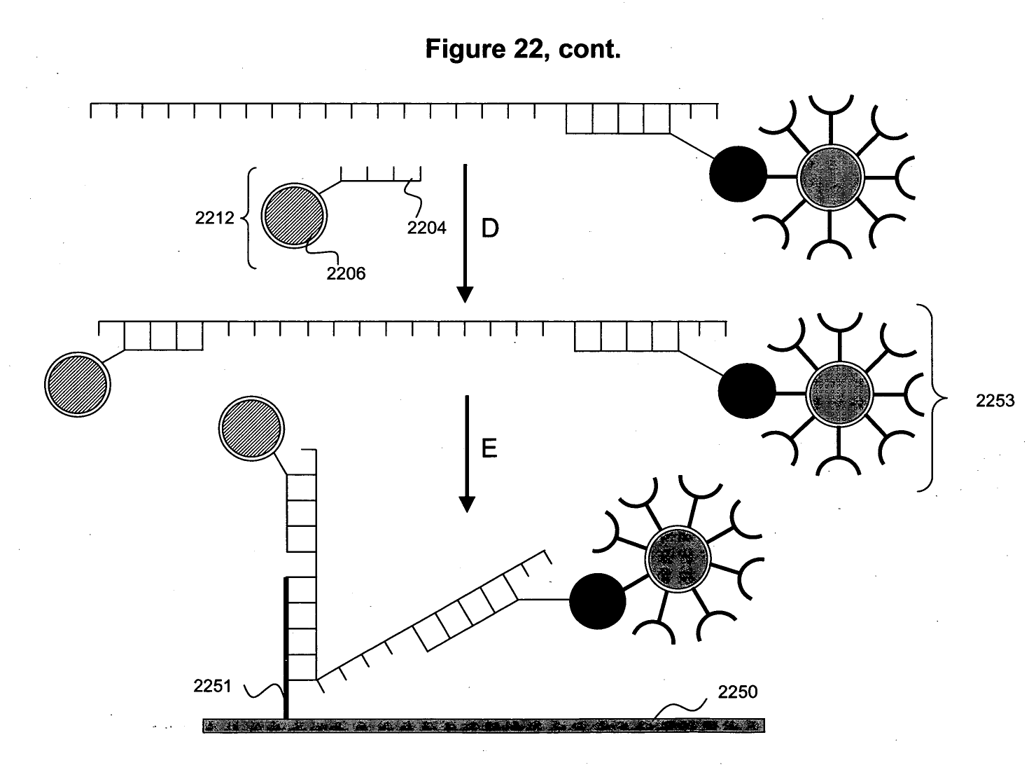

Certain embodiments of the invention use RFID tags in combination with enrichment of a target nucleic acid through magnetic capture in the detection of the target nucleic acid in a sample. In one such example is shown in FIG. 22, an oligo-reactive group complex (2235) comprises a reactive group (2237) and a first capture oligo (2239), which has a sequence that is substantially complementary to a first region of a target nucleic acid. Double-stranded target nucleic acid can be denatured (not shown) to produce the single-stranded form shown (2202). In step A, the oligo-reactive group complex (2235) is contacted with a sample suspected of containing target nucleic acid (2202) under conditions to, promote hybridization between the oligo-reactive group complex and the target nucleic acid. The hybridization of these entities forms hybridized reactive group complex (2241). In step B, an immobilized binding partner complex (2243) is contacted with the reaction mix. Immobilized binding partner complex (2243) comprises a magnetic particle (2247) and binding partners (2245) specific for the reactive group (2237) of the oligo-reactive group complex (2235). Therefore, binding of the immobilized binding partner complex (2243) to the oligo-reactive group complex immobilizes both unreacted oligo-reactive group complex (i.e., not bound to target nucleic acid) and hybridized reactive group complex (2241), which comprises oligo-reactive group complex (2235). An immobilized binding partner complex (2241) that has immobilized at least one hybridized reactive group complex (2241) is termed an immobilized hybridized complex (2249), whether or not an unreacted oligo-reactive group complex is also immobilized thereto. In alternative embodiments, reactive group (2237) is replaced with a magnetic particle, and immobilized binding partner complex (2243) is not required.

In step C, the reaction is subjected to a magnetic field (not shown), and the reaction is preferably washed or rinsed to remove target nucleic acid that did not hybridize with an oligo-reactive group complex to form a hybridized reactive group complex (2241). In step D, RFID-oligo complex (2212) is contacted with the reaction mix containing the immobilized hybridized complexes (2249). RFID-oligo complex (2212) comprises an RFID tag (2206) and a second capture oligo (2204) conjugated to or otherwise affixed to it. The second capture oligo (2204) of the RFID-oligo complex has a sequence that is substantially complementary to a second region of the target nucleic acid of the immobilized hybridized complex. The product of this reaction is RFID immobilized hybridized complex (2253). In certain embodiments, as illustrated in step E, RFID immobilized hybridized complex (2253) is contacted with, for example, a substrate (2250) that has an immobilized oligo (2251) affixed or otherwise attached to it. The immobilized oligo (2251) is designed to be substantially complementary to a third region of the target nucleic acid of RFID immobilized hybridized complex (2253). The substrate is preferably washed or rinsed to remove any RFID-oligo complexes that have not bound to the target nucleic acid to form an RFID immobilized hybridized complex (2253). If the target nucleic acid is present, RFID-oligo complexes that bound to the immobilized hybridized complexes to form RFID immobilized hybridized complexes that were immobilized on the substrate by hybridization of the immobilized oligo (2251) to the third region of the target nucleic acid will remain on the substrate (2250), and are subject to interrogation. Identification of a particular RFID tag is indicative of the presence of a particular target nucleic acid in the sample. In such instances where there is no target nucleic acid in the sample, the foregoing RFID immobilized hybridized complexes do not form, and no RFID tags are retained for detection on the substrate.

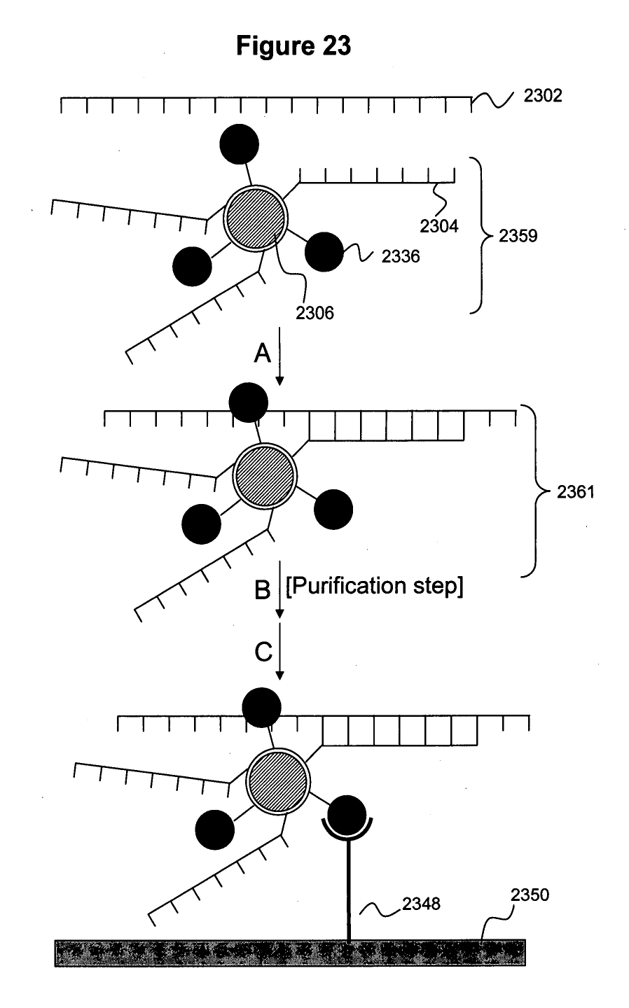

FIG. 23 illustrates another use of RFID tags the present invention. An RFID tag (2306) has a capture oligo (2304) and a reactive group (2336) conjugated to or otherwise affixed to it to form RFID-oligo-reactive group complex (2359). The capture oligo (2304) of the RFID-oligo-reactive group complex (2359) is designed to have a sequence substantially complementary to a target nucleic acid. Double-stranded target nucleic acid can be denatured (not shown) to produce the single-stranded target nucleic acid (2302) shown. In step A, the RFID-oligo-reactive group complex (2359) is contacted with a sample suspected of containing target nucleic acid (2302) under conditions to promote hybridization between the RFID-oligo-reactive group complex and target nucleic acid. The hybridization of these entities forms hybridized RFID-oligo-reactive group complex (2361). In step B, a purification procedure is carried out to separate hybridized RFID-oligo-reactive group complex (2361) from any RFID-oligo-reactive group complex (2359) that did not associate with target nucleic acid. For example, if the target nucleic acid is DNA and the capture oligo is RNA, the reaction mixture may be passed over a column comprising antibodies that specifically bind to DNA-RNA hybrids, thereby immobilizing the hybridized RFID-oligo-reactive group complex (2361) and allowing removal of the RFID-oligo-reactive group complex (2359) that did not associate with target nucleic acid. Other purification methods are well known to those of skill in the art. In certain embodiments, as illustrated in step C, hybridized RFID-oligo-reactive group complex (2361) can be contacted with, for example, a substrate (2350) with an immobilized binding partner (2348) affixed to or otherwise associated with the substrate. Immobilized binding partner (2348) is designed to preferentially bind to the reactive group (2336), thereby immobilizing, e.g., hybridized RFID-oligo-reactive group complex (2361). The substrate is preferably washed or rinsed to remove any oligo-reactive group-RFID complexes that have not formed into an RFID-oligo-reactive group complex (i.e., unreacted oligo-reactive group-RFID complexes). The substrate can be subjected to RFID interrogation (not shown). If the target nucleic acid is present, the reacted oligo-reactive group-RFID complexes (having formed with the target nucleic acid into RFID complexes) will remain on the substrate, and are subject to interrogation. Identification of a particular RFID tag will be indicative of the presence of the target nucleic acid in the sample. In such instances where there is no target nucleic acid in the sample, the foregoing RFID complex will not form, and no RFID signal will be retained for detection.

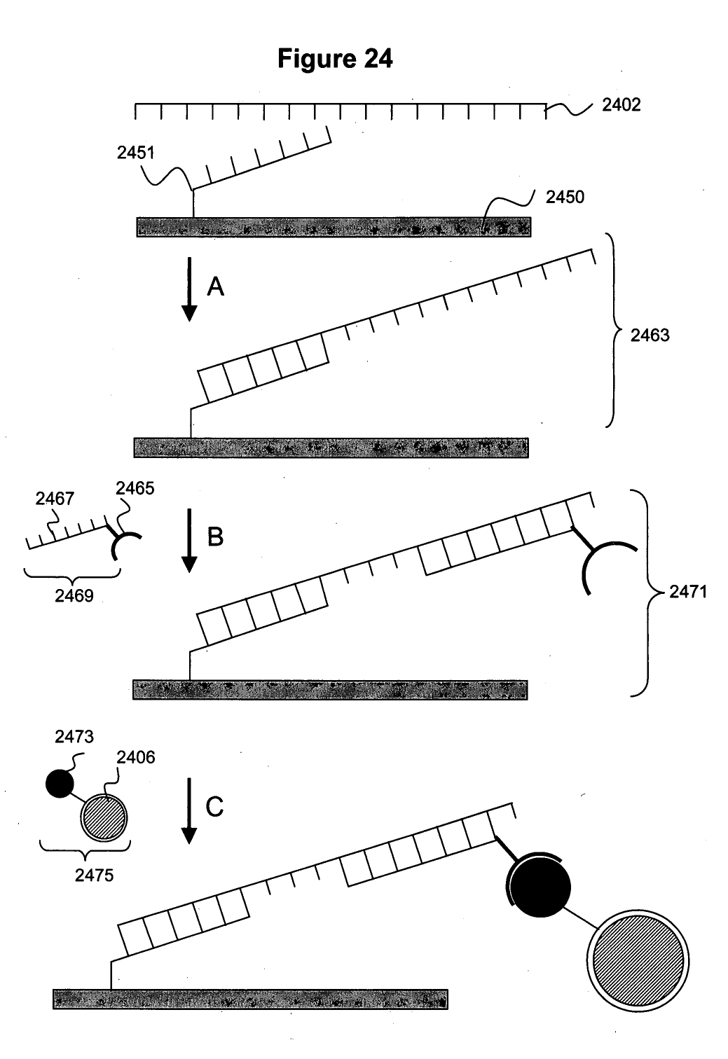

In certain embodiments, RFID tags may be used in combination with reactive groups in the detection of nucleic acid in a sample. For example, FIG. 24 illustrates target nucleic acid (2402) and a substrate (2450) with an immobilized nucleic acid (2451) affixed to or otherwise associated with the substrate. Double-stranded target nucleic acid can be denatured (not shown) to produce the single-stranded target nucleic acid (2402) shown. Immobilized nucleic acid (2451) has a sequence substantially complementary to a first region of target nucleic acid (2402). In step A, a sample suspected of containing target nucleic acid (2402) can be contacted with, for example, a substrate (2450) associated with immobilized nucleic acid (2451) under conditions to promote hybridization between the target nucleic acid (2402) and the immobilized nucleic acid (2451). The hybridization of these two entities produces immobilized target nucleic acid complex (2463). In step B, oligo-binding partner complex (2469) is mixed or contacted with the reaction mix. Oligo-binding partner complex (2469) comprises a capture oligo (2467), which is substantially complementary to a second region of target nucleic acid, and a binding partner (2465). The addition of oligo-binding partner complex (2469) to the reaction mix under conditions to promote hybridization between the target nucleic acid (2402) and the oligo-binding partner complex (2469) yields target nucleic acid-binding partner complex (2471). Optionally, the substrate (2450) may be washed or rinsed to remove any oligo-binding partner complex (2469) that did not bind to an immobilized target nucleic acid complex (2463). In certain embodiments, as illustrated in step C, RFID-reactive group complex (2475) is added to the reaction mix containing target nucleic acid-binding partner complex (2471). RFID-reactive group complex (2475) comprises an RFID tag (2406) with a reactive group (2473) affixed to or otherwise bound to it. Reactive group (2473) is designed to preferentially bind to binding partner (2465), thereby bringing RFID tag (2406) into close proximity with substrate (2450) when binding partner (2465) is part of a target nucleic acid-binding partner complex (2471). The substrate is preferably washed or rinsed to remove any RFID-reactive group complexes that have not bound to a target nucleic acid-binding partner complex. The substrate can be subjected to RFID interrogation (not shown). If the target nucleic acid is present, reacted RFID-reactive group complexes (having bound to the binding partner (2465) on the target nucleic acid-binding partner complex (2471)) remain on the substrate, and are subject to interrogation. Identification of a particular RFID tag is indicative of the presence of the target nucleic acid in the sample. In such instances where there is no target nucleic acid in the sample, the foregoing complex do not form, and no RFID signal is retained for detection.

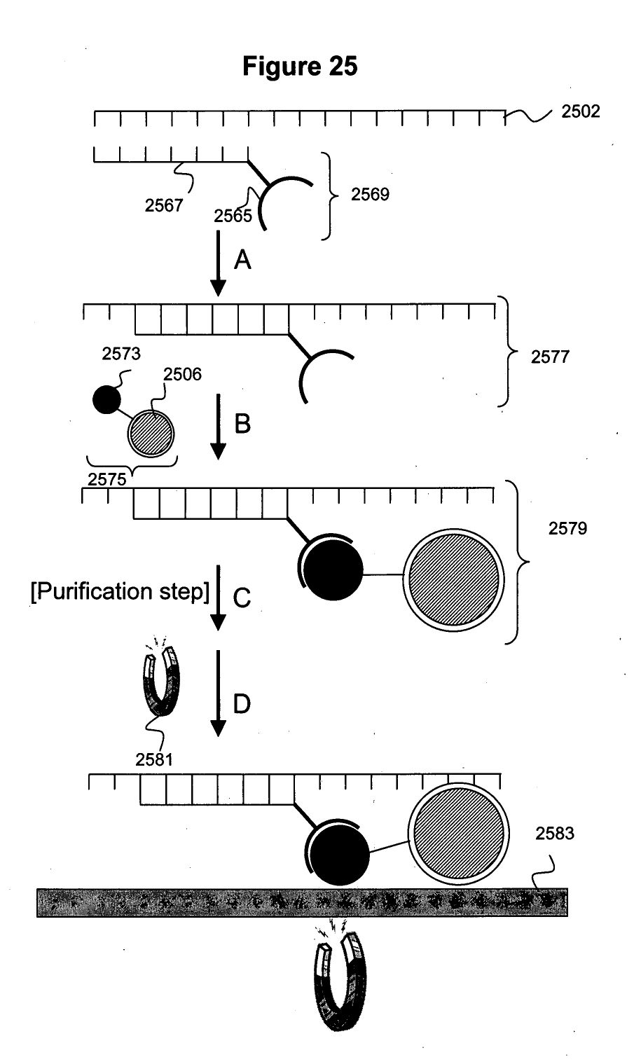

In certain embodiments, RFID tags may be used in combination with magnetized reactive groups in the detection of nucleic acid in a sample. FIG. 25 illustrates one such embodiment. Double-stranded target nucleic acid can be denatured (not shown) to produce the single-stranded target nucleic acid (2502) shown. In step A, a sample suspected of containing target nucleic acid (2502) is mixed or contacted with oligo-binding partner complex (2569), which comprises a capture oligo (2567) that is substantially complementary to a region of target nucleic acid (2502), under conditions that promote hybridization between the two entities to form hybridized binding partner complex (2577). Oligo-binding partner complex, and therefore also hybridized binding partner complex, further comprises a binding partner (2565) (e.g., an antibody). In certain embodiments, as illustrated in step B, RFID-reactive group complex (2575) is added to the reaction mix containing hybridized binding partner complex (2577) to form RFID-reactive group-binding partner complex (2579). RFID-reactive group complex (2575) comprises an RFID tag (2506) and a magnetized reactive group (2573), which has a magnetic core and a reactive group that preferentially binds to binding partner (2565). In step C, a purification procedure is carried out to separate RFID-reactive group-binding partner complex (2579) from any RFID-reactive group complex (2575) that did not associate with a hybridized binding partner complex (2577). For example, if the target nucleic acid is DNA and the capture oligo is RNA, the reaction mixture may be passed over a column comprising antibodies that specifically bind to DNA-RNA hybrids, thereby immobilizing the RFID-reactive group-binding partner complex (2579) and allowing removal of the RFID-reactive group complex (2575) that did not associate with a hybridized binding partner complex (2577). Other purification methods are well known to those of skill in the art. In certain embodiments, as illustrated in step D, RFID-reactive group-binding partner complex (2579) can be subjected to a magnetic field (2581) that will draw the RFID-reactive group-binding partner complex (containing the magnetic particle of magnetized reactive group-RFID (2573) toward the magnetic field. In practice, the reaction may be performed in a reaction container such as a test tube (not shown). Application of the magnetic field (2581) on a side of test tube will draw the magnetized RFID-reactive group-binding partner complexes (2579) to the side of the test tube wall (2583) most proximate to the magnetic field. The reaction is preferably washed or rinsed, one or more times, and perhaps multiple times, and the reaction can be subjected to RFID interrogation (not shown). If the target nucleic acid is present, the RFID tags in the RFID-reactive group-binding partner complexes are subject to interrogation. Identification of a particular RFID tag is indicative of the presence of the target nucleic acid in the sample. In such instances where there is no target nucleic acid in the sample, the foregoing complex does not form, and no RFID signal is retained for detection.

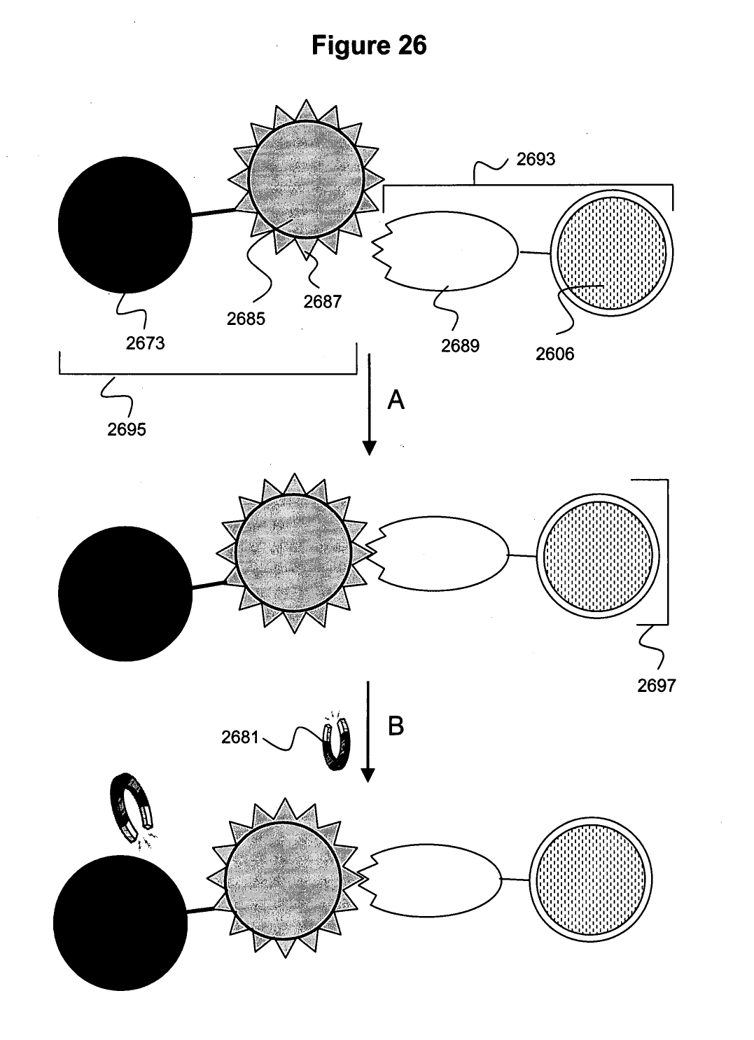

In some embodiments, RFID complexes may be used in the detection of cell surface carbohydrate structures in a sample. In one aspect of this embodiment, an RFID tag with a capture moiety attached to or otherwise associated with it is mixed or contacted with magnetic particles associated with or attached to cells suspected of containing a given surface carbohydrate structure. FIG. 26 illustrates one such embodiment in which a magnetic particle (2673) has a cell (2685) affixed to or otherwise attached to it to form cell-magnetic particle complex (2695). Cell (2685) is shown with surface carbohydrate structures (2687). RFID tag (2606) is affixed to or otherwise associated with a capture moiety (2689) to form RFID-capture moiety complex (2693). Capture moiety (2689) is designed to preferentially bind to surface carbohydrate structures (2687). In step A, cell-magnetic particle complex (2695) and RFID-capture moiety complex (2693) are mixed or contacted with each other under conditions such that if a surface carbohydrate structure (2687) is present on cell-magnetic particle complex (2695), capture moiety (2689) of RFID-capture moiety complex (2693) will preferentially bind to it to form reacted RFID complex (2697). The capture moiety used may include, but is not limited to, a lectin. In certain embodiments, as illustrated in step B, a reacted RFID complex (2697) can be subjected to a magnetic field (2681) which will draw the reacted RFID complex (containing the magnetic particle) toward the magnetic field. In practice, the reaction may be performed in a reaction container such as a test tube (not shown). Application of the magnetic field (2681) on a side of a test tube, for example, will draw the magnetized reacted RFID complexes (2697) to the side of the test tube wall most proximate to the magnetic field. The reaction is preferably washed one or more times to remove any RFID-capture moiety complexes (2693) not bound to cell-magnetic particle complexes (2695), and the reaction can then be subjected to RFID interrogation (not shown). In such instances where there are no surface carbohydrate structures on the cells in the sample bound by the capture moiety (2689), the foregoing reacted RFID complex (2697) does not form, and no RFID tag is retained for detection.

As stated, the capture moiety used may include, but is not limited to, lectins. A representative, non-limiting list of lectins that may be used is listed in Table 1. Selectins also bind to carbohydrate groups and may similarly be used as capture moieties. The assay may also be multiplexed using RFID complexes with different capture moieties, and different binding pairs may be used as the capture moiety and target agent. By way of a non-limiting example, the avian and human flu viruses have a surface glycoprotein hemagglutinin that has a strong affinity to terminal sialic acid. Detection of these flu viruses may be accomplished using a RFID-capture moiety complex as described above where the capture moiety comprises terminal sialic acid.

TABLE 1
Representative Lectins (in addition to the
various genetic sequences coding thereof)

| Plant Species | Lectin | Specificity |
|---|---|---|
| Arachis hypogaea | peanut agglutinin | beta-D-galactose |
| Canavalia ensiformis | convanavalin A | alpha-D-glucose; alpha-D- |
| | | mannose |
| Dolichus biflorus | Dolichus biflorus | N-acetyl-alpha-D- |
| | agglutinin | galactosamine |
| Glycine max | soy bean agglutinin | N-acetyl-alpha-D- |
| | | galactosamine; beta-D- |
| | | galactose |
| Lens culinaris | Lens culinaris | alpha-D-glucose; alpha-D- |
| | agglutinin | mannose |
| Phaseolus vulgaris | Phaseolus | N-acetyl-alpha-D- |
| | agglutinin 1 | galactosamine |
| Pisum sativum | pealectin-1 | alpha-D-glucose; alpha-D- |
| | | mannose |
| Ricinus communis | Ricinus communis | beta-D-galactose; N-acetyl- |
| | agglutinin | alpha-D-galactosamine |
| Triticum vulgare | wheat germ | (N-acetyl-beta-(1-4)-D- |
| | agglutinin | glucosamine); chitin; |
| | | chitotriose |
| Ulex europaeus | Ulex europaeus | alpha-L-fucose |
| | agglutinin | |

In certain embodiments of the present invention, a biomolecular-complexed RFID is contacted with a metal and/or metal-containing compound (e.g., a “metallo compound”) that can associate with the biomolecules (such as DNA, or organic molecules such as a porphyrin molecule or other cyclic organic structures). For example, double-stranded target nucleic acid-oligonucleotide species on an RFID tag can be associated with a metal-containing compound and serve as an RFID tag's radio frequency antenna. In some embodiments, the coiled conformation of double-stranded nucleic acid may serve to enhance the signaling between an RFID tag and an RFID reader. In some embodiments, the existence of double-stranded nucleic acid may be required for any signaling to occur between an RFID tag and an RFID reader. In some embodiments, a metallo-compound(s) is used which associates only with double-stranded nucleic acid. Common types of association (e.g., between a metal-containing compound and a nucleic acid) include, but are not limited to, intercalation, complexation, minor groove binding and/or association, major groove binding and/or association, covalent and non-covalent interactions. The metallo compounds of the present invention can be used in a cooperative manner with other molecules which have the effect or potential effect of enabling, enhancing or otherwise affecting the association of the metal-containing compound with the nucleic acid molecule(s). In some embodiments, the association of the metal-containing compound changes or alters the characteristics of the antenna and/or the RFID signal.

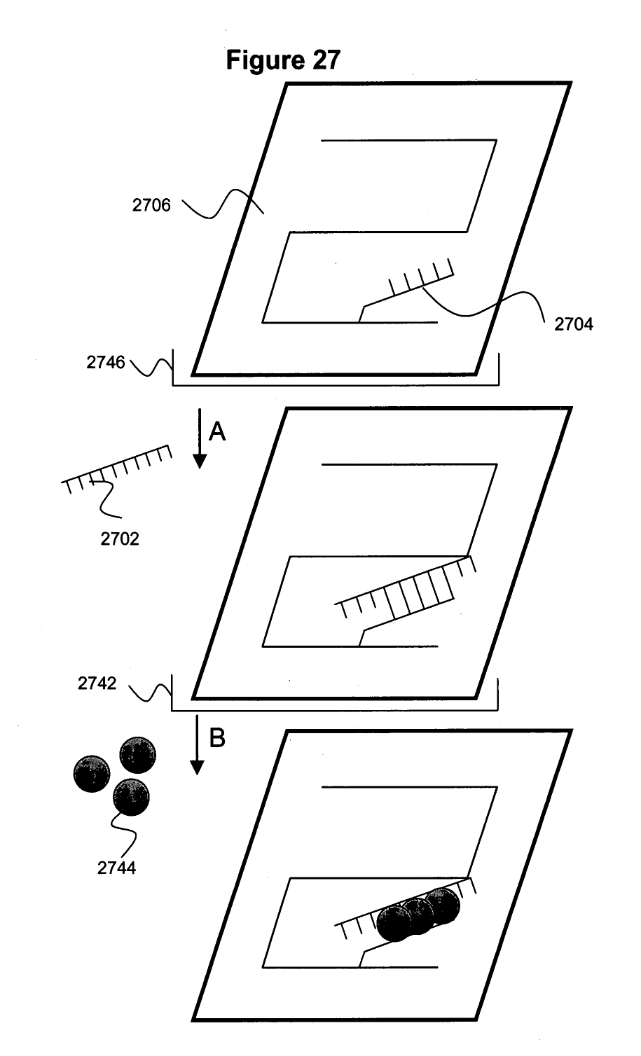

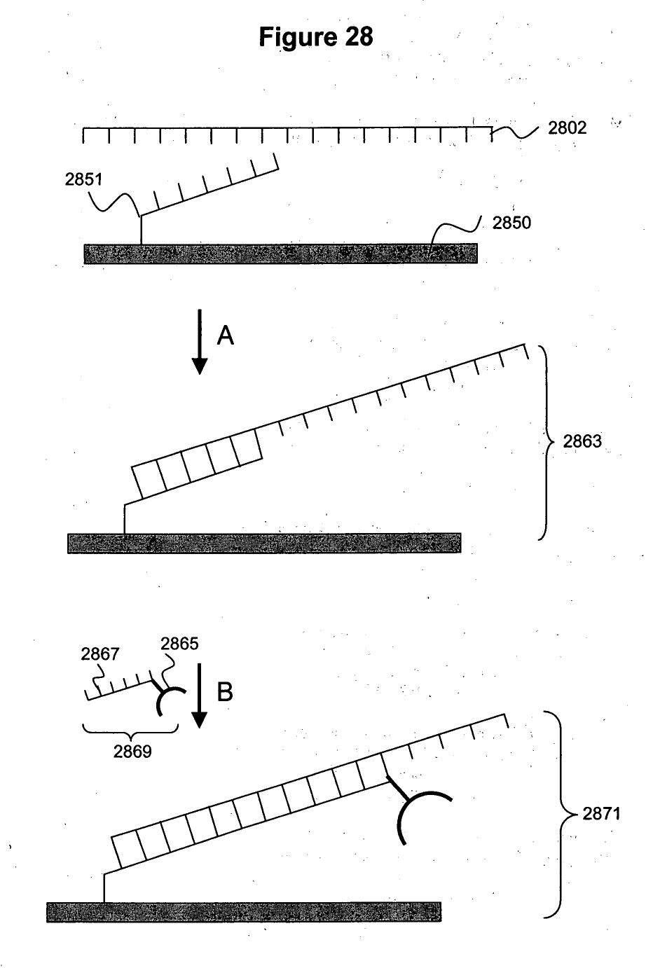

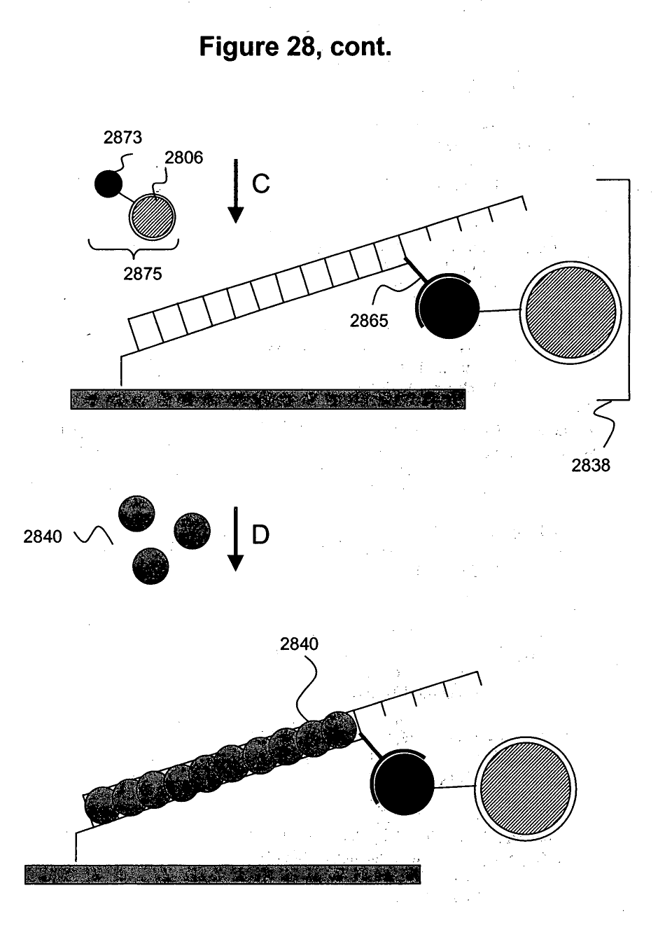

One aspect of this embodiment is illustrated in FIG. 27, which illustrates an embodiment where an RFID tag is contacted with a metal and/or metal-containing compound (e.g., a metallo compound). An oligonucleotide (2704) is conjugated to or otherwise associated with an RFID tag (2706) to form RFID-oligo complex (2746). The oligonucleotide (2704) is designed to have a sequence substantially complementary to at least a portion of a target nucleic acid (2702). In step A, RFID-oligo complex (2746) is mixed or contacted with a sample suspected of containing a target nucleic acid (2702) under conditions to promote hybridization between the target nucleic acid and the oligo of the RFID-oligo complex. If the target nucleic acid is present, the target nucleic acid will hybridize to the oligo of RFID-oligo complex to form reacted RFID complex (2742), which comprises a region of double-stranded nucleic acid. In some embodiments, as illustrated in step B, a metal-containing compound (2744) (which can be added before, during, or after hybridization occurs) is allowed contact with the reacted RFID complex (2742) under conditions such that the metal-containing compound can associate with the double-stranded nucleic acid species on the reacted RFID complex, essentially creating an antenna with which to transmit an RFID signal from the RFID tag. The reaction is subjected to RFID interrogation. In such instances where no target nucleic acid is present, the foregoing metal-containing compound-reacted RFID complex will not form, no antenna is created with which to transmit an RFID signal, and no RFID signal is detected. FIG. 28 illustrates another use of the RFID tags and metal-containing compounds in the detection of nucleic acid in a sample. This embodiment uses a target nucleic acid (2802) and a substrate (2850) with an immobilized nucleic acid (2851) affixed to or otherwise associated with the substrate. Double-stranded target nucleic acid can be denatured (not shown) to produce the single-stranded target nucleic acid (2802) shown. Immobilized nucleic acid (2851) is designed to have a sequence substantially complementary to a first portion target nucleic acid (2802). In step A, a sample suspected of containing target nucleic acid can be contacted with, for example, a substrate (2850) with the associated immobilized nucleic acid (2851) under conditions to promote hybridization between the target nucleic acid and the immobilized nucleic acid. The hybridization of these two entities produces immobilized target nucleic acid complex (2863), which comprises a region of double-stranded nucleic acid. In step B, oligo-binding partner complex (2869) is mixed or contacted with the reaction mix. Oligo-binding partner complex (2869) comprises an oligo (2867) which is substantially complementary to a second portion of target nucleic acid and a binding partner (2865). The addition of oligo-binding partner complex (2869) to the reaction mix yields immobilized target nucleic acid-binding partner complex (2871), which comprises more double-stranded nucleic acid than does immobilized target nucleic acid complex (2863). Although FIG. 28 shows oligo (2867) hybridizing to the target nucleic acid adjacent to oligo (2851), this is not required and oligo (2867) may hybridize to a second region of the target nucleic acid that is not adjacent to the region to which oligo (2851) hybridizes. In certain embodiments, as illustrated in step C, RFID-reactive group complex (2875) is added to the reaction mix containing immobilized target nucleic acid-binding partner complex (2871) to yield reacted RFID complex (2838). RFID-reactive group complex (2875) comprises an RFID tag (2806) with a reactive group (2873) affixed to or otherwise bound to it. Reactive group (2873) is designed to preferentially bind to binding partner (2865). In certain embodiments, as illustrated in step D a metal-containing compound (which can be added before, during, or after hybridization occurs) is allowed contact with reacted RFID complex (2838) under conditions such that the metal-containing compound can associate with the double-stranded nucleic acid portion of the immobilized target nucleic acid complex of the reacted RFID complex, essentially creating an antenna with which to transmit an RFID signal from the RFID tag (2806). The reaction is then subject to RFID interrogation (not shown). In such instances where no target nucleic acid is present, the foregoing reacted RFID complex does not form, and no RFID tag is retained for detection.

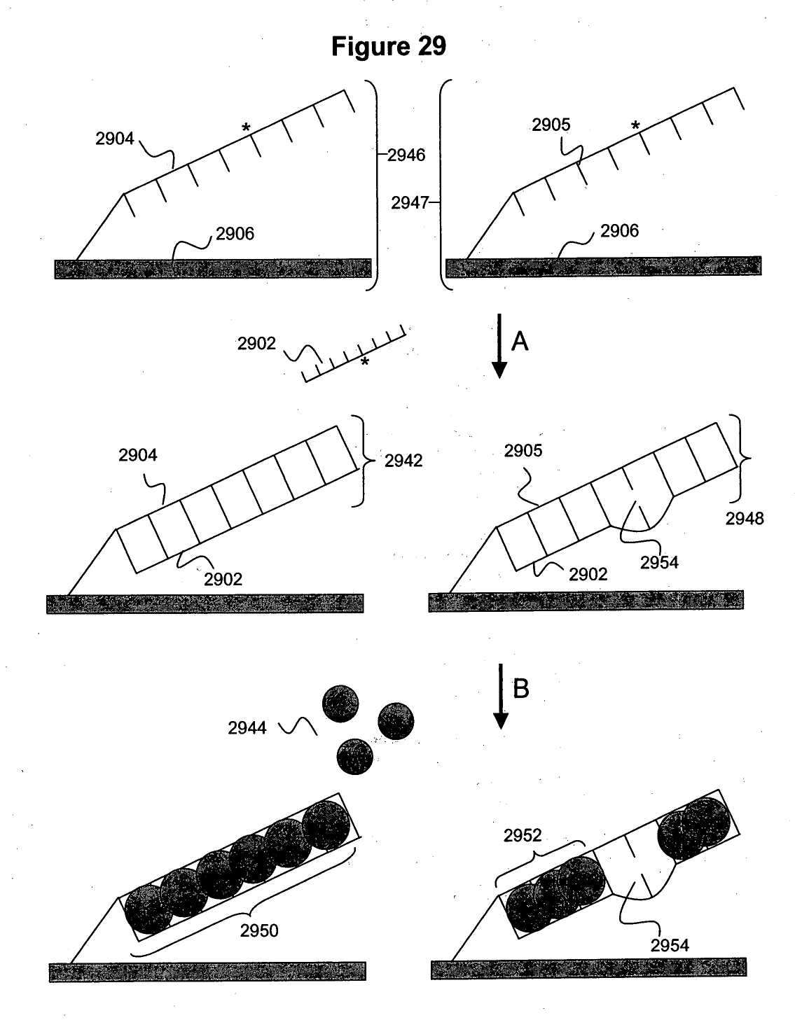

FIG. 29 illustrates another embodiment for the detection of SNPs in target nucleic acid where an RFID tag is contacted with a metal and/or metal-containing compound (e.g., metallo compound). A first RFID-oligo complex (2946) comprising an oligo (2904) and an RFID tag (2906) and a second RFID-oligo complex (2947) comprising an oligo (2905) and an RFID tag (2906) are shown. The oligos (2904 and 2905) have sequences that are identical except at a single position (indicated by an asterisk), where they differ by a single nucleotide. Oligo (2904) is substantially complementary to a target nucleic acid (2902) comprising a SNP position (indicated by an asterisk), and oligo (2905) is substantially complementary to the target nucleic acid (2902) except at the SNP position. In step A, RFID-oligo complexes (2946 and 2947) are mixed or contacted with a sample suspected of containing target nucleic acid (2902) under conditions to promote hybridization between the target nucleic acid and the oligos of the RFID-oligo complexes. Hybridized RFID complex (2942) is formed when target nucleic acid (2902) has basepair complementarity with the oligo (2904) of RFID-oligo complex (2946), and mismatch RFID complex (2948) is formed when target nucleic acid (2902) has a basepair mismatch (2954) when hybridized to the oligo (2905) of RFID-oligo complex (2947). In some embodiments, as illustrated in step B, a metal-containing compound (2944) (which can be added before, during, or after hybridization occurs) is allowed contact with hybridized RFID complex (2942) and/or mismatch RFID complex (2948) under conditions such that the metal-containing compound can associate with the double-stranded regions of the complexes formed by hybridization of the target nucleic acid (2902) with the oligos (2904 and/or 2905, respectively), essentially creating an antenna with which to transmit an RFID signal from the respective RFID tag. When there is no mismatch, a long antenna can form, such as antenna (2950), which extends all or part of the length of the double-stranded region of the hybridized RFID complex (2942). When there is a mismatch, a short antenna forms, such as antenna (2952), which extends all or part of the length of the double-stranded region of the mismatch RFID complex (2948), but does not extend through the mismatch region (2954). The antenna length is directly proportional to the RFID tag's radio frequency operating wavelength. Thus, long antenna (2950) will have a different radio frequency operating wavelength than short antenna (2952). The reaction is subjected to RFID interrogation at one or perhaps more than one radio frequency operating wavelength. Thus, the genotype of the SNP position in a target nucleic acid in a sample can be determined based on the known sequence of the oligo hybridized to the target nucleic acid. For example, a long antenna is indicative that the oligo hybridized to the target nucleic acid has no mismatches and is therefore complementary at the SNP position, and detection of a short antenna is indicative that the oligo hybridized to the target nucleic acid has at least one mismatch, suggesting that it is not complementary at the SNP position.

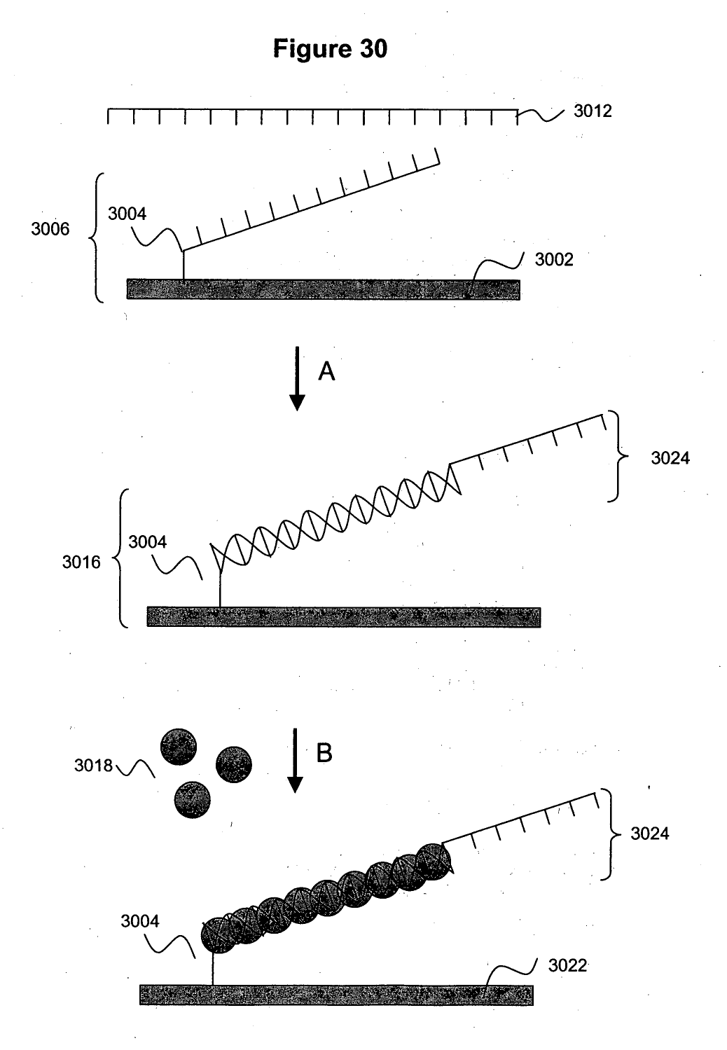

FIG. 30 illustrates another embodiment where an RFID tag is contacted with a metal and/or metal-containing compound (e.g., metallo compound). An oligo (3004) is conjugated to or otherwise associated with an RFID tag (3002) to form RFID-oligo complex (3006). The oligo (3004) is designed to have a sequence substantially complementary to at least a region of a target nucleic acid (3012). In step A, RFID-oligo complex (3006) is mixed or contacted with a sample suspected of containing target nucleic acid (3012) under conditions to promote hybridization between the target nucleic acid and the oligo of the RFID-oligo complex. If the target nucleic acid is present, the target nucleic acid will hybridize to the oligo of the RFID-oligo complex to form a reacted RFID complex (3016) comprising a region of double-stranded nucleic acid. In some embodiments, as illustrated in step B, a metal-containing compound (3018) (which can be added before, during, or after hybridization occurs) is allowed contact with a reacted RFID complex (3016) under conditions such that the metal-containing compound can associate with the double-stranded region of the reacted RFID complex formed by hybridization of the target nucleic acid with the oligo (3004), essentially creating an antenna with which to transmit an RFID signal. The reaction is subjected to RFID interrogation. In such instances where no target nucleic acid is present, no antenna is created with which to transmit an RFID signal, and no RFID signal is detected.

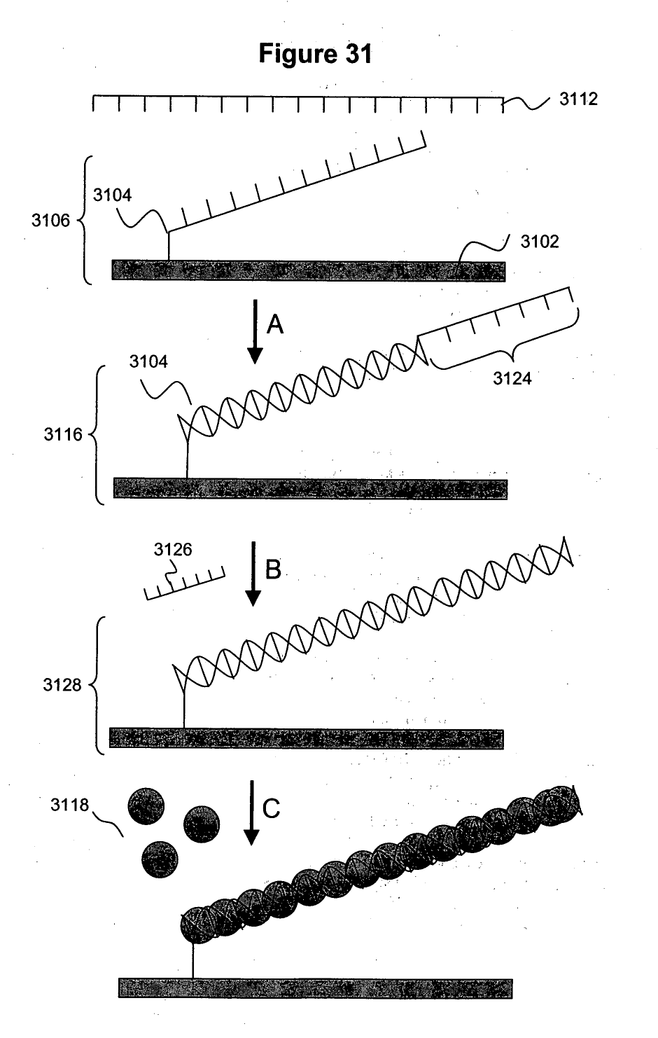

FIG. 31 illustrates another embodiment where an RFID tag is contacted with a metal and/or metal-containing compound (e.g., metallo compound). An oligo (3104) is conjugated to or otherwise associated with an RFID tag (3102) to form RFID-oligo complex (3106). The oligo (3104) is designed to have a sequence substantially complementary to at least a region of a target nucleic acid (3112). In step A, RFID-oligo complex (3106) is mixed or contacted with a sample suspected of containing target nucleic acid (3112) under conditions to promote hybridization between the target nucleic acid and the oligo of the RFID-oligo complex. If the target nucleic acid is present, the target nucleic acid will hybridize to the oligo of the RFID-oligo complex to form a reacted RFID complex (3116), which comprises a region of double-stranded nucleic acid. Also shown is an overhang portion (3124) of the target nucleic acid. In some embodiments, as illustrated in step B, an oligo (3126) is mixed or contacted with the reaction mix under conditions to promote hybridization between the oligo (3126) and the overhang portion (3124) of the target nucleic acid to form elongated reacted RFID complex (3128), which comprises more double-stranded nucleic acid than does reacted RFID complex (3116). Oligonucleotide (3126) is designed to have a sequence substantially complementary to overhang portion (3124) of the target nucleic acid. In some embodiments, as illustrated in step C, a metal-containing compound (3118) (which can be added before, during, or after hybridization occurs) is allowed contact with the elongated reacted RFID complex (3128) under conditions such that the metal-containing compound can associate with the double-stranded region of the elongated reacted RFID complex formed by the hybridization of the target nucleic acid with oligos (3104 and 3126), essentially creating an antenna with which to transmit an RFID signal. The reaction is subjected to RFID interrogation. In such instances where no target nucleic acid is present, no antenna is created with which to transmit an RFID signal, and no RFID signal is detected. Alternatively, a partially double-stranded target nucleic acid may be mixed or contacted with the RFID-oligo complex (3106), with the portion of the target nucleic acid that is to bind with the oligo of the RFID-oligo complex being single-stranded and the region indicated as the overhang portion (3124) being double-stranded, in which case oligo (3126) need not be hybridized to the target nucleic acid prior to association with the metal-containing compound (3118). In certain other embodiments, the overhang portion (3124) comprises a SNP position and oligo (3126) is complementary to one genotype of the SNP. Therefore, binding of oligo (3126), or lack thereof, is indicative of the genotype of the SNP. Addition of the metal-containing compound (3118) will allow formation of a long antenna if oligo (3126) is complementary at the SNP position, or a short antenna if oligo (3126) is not complementary at the SNP position. As described above, a long antenna will have a different radio frequency operating wavelength than a short antenna. The reaction is subjected to RFID interrogation at one or perhaps more than one radio frequency operating wavelength to determine whether a long or short antenna formed, and therefore the genotype at the SNP position.

Metallo-compounds useful in the present invention can be, for example, a metallo-intercalating agent characterized by a tendency to intercalate specifically into double-stranded nucleic acid such as double-stranded DNA. These intercalating agents have in their molecules a flat intercalating group such as a phenyl group, which intercalates between the base pairs of the double-stranded nucleic acid, therefore binding to the double-stranded nucleic acid. Metallo-containing intercalating agents useful in the present invention include, but are not limited to, ferritin, ethidiumcis-platin, tris(phenanthroline)zinc salt, tris(phenanthroline)ruthenium salt, tris(phenantroline)cobalt salt, di(phenanthroline)zinc salt, di(phenanthroline)ruthenium salt, di(phenanthroline)cobalt salt, bipyridine platinum salt, terpyridine platinum salt, phenanthroline platinum salt, tris(bipyridyl)zinc salt, tris(bipyridyl)ruthenium salt, tris(bipyridyl)cobalt salt, di(bipyridyl)zinc salt, di(bipyridyl)ruthenium salt, di(bipyridyl)cobalt salt, and the like.

Transition metals are those whose atoms have a partial or complete d orbital shell of electrons. Suitable transition metals for use in conjunction with the present invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), Vanadium (V), chromium (Cr), manganese (Mn), nickel (Ni), molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinum, cobalt and iron.

The transition metals may be complexed with a variety of ligands, to form suitable transition metal complexes. As will be appreciated by those in the art, the number and nature of the co-ligands will depend on the coordination number of the metal ion. Mono-, di- or polydentate co-ligands may be used at any position. Suitable ligands fall into two categories: ligands, which use nitrogen, oxygen, sulfur, carbon or phosphorus atoms (depending on the metal ion) as the coordination atoms (generally referred to in the literature as sigma (Σ) donors) and organometallic ligands such as metallocene ligands (generally referred to in the literature as pi (π) donors).

Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp 73-98), 21.1 (pp. 813-898) and 21.3 (pp 915-957), all of which are hereby expressly incorporated by reference.

Suitable sigma donating ligands using carbon, oxygen, sulfur, and phosphorus are known in the art. For example, suitable sigma carbon donors are found in Cotton and Wilkinson, Advanced Organic Chemistry, 5th Edition, John Wiley & Sons (1988), hereby incorporated by reference; see, e.g., page 38. Similarly, suitable oxygen ligands include crown ethers, water and others known in the art. Phosphines and substituted phosphines are also suitable; see, e.g., page 38 of Cotton and Wilkinson. The oxygen, sulfur, phosphorus and nitrogen-donating ligands are attached in such a manner as to allow the heteroatoms to serve as coordination atoms.

Such organometallic ligands include cyclic aromatic compounds such as the cyclopentadienide ion [C₅H₅ (−1)] and various ring substituted and ring fused derivatives, such as the indenylide (−1) ion, that yield a class of bis(cyclopentadieyl) metal compounds, (i.e., the metallocenes); see, e.g., Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples, which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to the nucleic acid. Other potentially suitable organometallic ligands include cyclic arenes such as benzene, to yield bis(arene)metal compounds and their ring substituted and ring fused derivatives, of which bis(benzene)chromium is a prototypical example. Other acyclic pi-bonded ligands such as the allyl(−1) ion, or butadiene yield potentially suitable organometallic compounds, and all such ligands, in conjunction with other pi-bonded and delta-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene is derivatized.

Alternatively, in some embodiments, the nucleic acid may be labeled with an electroactive marker. Such electroactive markers can include, but are not limited to, ferrocene derivatives, anthraquinone, silver and silver derivatives, gold and gold derivatives, osmium and osmium derivatives, ruthinium and ruthinium derivatives, cobalt and cobalt derivatives and the like.

In other embodiments, the duplex nucleic acid can be associated with Zn²⁺, Ni²⁺, or Co²⁺ to form another metal complex, called M-DNA. See, e.g., Lee et al., Nucleic Acids Research, 30:2244 (2002). M-DNA has the property of allowing electron transfer through the nucleic acid helix, and therefore may serve as a metallic nucleic acid conductor.

In other embodiments, the metallo compound is used in conjunction with RFID associated with chemical moieties, biological molecule, biological systems or other structure that is capable of interacting or which interacts with said metallo compound. For example, the RFID can be associated with (either before or after being contacted with the desired target entity) an enzyme or other reactive molecule which requires and/or is otherwise affected by the presence of a metal/metallo cofactor or other interaction involving a metallo compound. For example, the complexation or other association with the metallo compound can affect the RFID signal to be detected. In addition, the presence of the metallo compound, can affect the ability for a detectable complex to be formed and/or enable the dissociation and/or other transformation of the detectable complex.

In certain embodiments, the association of the metallo compound functions as an antenna for the associated RFID, thereby enabling the detection of the RFID. In other embodiments, the association of the metallo compound affects the one or more characteristics of the RFID signal including, inter alia, the strength of the signal (increasing or decreasing), the frequency, the wavelength, or other detectable and/or potentially detectable characteristic.

In some embodiments, methods utilizing RFID interrogation may be used in combination with other methods of detecting a target agent in a sample. For example, secondary confirmation of a binding event between a loaded RFID complex and a target agent or an immobilized binding partner may be done through such means as electrochemical detection or fluorescence. Use of electrochemical detection is described in detail, for example, in the co-pending application U.S. Ser. No. 60/850,016, filed Oct. 6, 2006, entitled “Scaffold-Bound Capture Moieties and Uses Thereof,” and U.S. Ser. No. 11/703,103, filed Feb. 7, 2007, entitled “Device and Methods for Detecting and Quantifying One or More Target Agents,” both of which are hereby incorporated by reference. In one aspect of one embodiment of the invention, capture-associated oligos are conjugated to a loaded RFID complex (comprising a capture moiety) to form an loaded RFID-oligo complex, and the target agent of interest is an antigen. (A capture-associated oligo is an oligo associated with a capture moiety, whether the association is direct or indirect, e.g., via a scaffold or RFID device.) In accordance with this embodiment the invention the following elements are included, in varied orders or combinations: (1) an electrode-associated oligo immobilized on a surface, where the surface comprises an electrode, (2) a loaded RFID-oligo complex comprising a capture-associated oligo that is complementary to the electrode-associated oligo, an RFID tag, and a capture moiety specific for the target agent, (3) immobilized binding partners, and (4) a sample suspected of containing the target antigen. After the loaded RFID-oligo complex is contacted with the sample to form a first mixture, the first mixture is contacted with the immobilized binding partners. The unreacted loaded RFID-oligo complexes are captured by the immobilized binding partners and the reacted loaded RFID-oligo complexes are left in solution, thereby separating the unreacted loaded RFID-oligo complexes from the reacted loaded RFID-oligo complexes. The reacted loaded RFID-oligo complexes may be scanned to determine which RFID tags are present in solution, which serves as a first indication as to the presence of the target agent in the sample. In various aspects of this embodiment, the capture-associated oligos associated with the reacted loaded RFID-oligo complexes may undergo optional cleavage reactions and/or amplification via linear or logarithmic methods known in the art and further described in, e.g., U.S. Ser. No. 11/703,103, filed Feb. 7, 2007, entitled “Device and Methods for Detecting and Quantifying One or More Target Agents,” incorporated herein by reference in its entirety for all purposes. Typically such treatment is performed after the reacted loaded RFID-oligo complexes are separated from the unreacted loaded RFID-oligo complexes, but before being contacted with the electrode-associated oligos. Subsequently, the solution phase of the mixture is contacted with the electrode-associated oligos. Electrochemical detection will reveal whether capture-associated oligos (or oligos amplified therefrom) are hybridized to electrode-associated oligos, which serves as a second indication as to the presence of the target agent in the sample. In alternative embodiments, the reacted loaded RFID-oligo complexes can be immobilized with the immobilized binding partners as described elsewhere herein, leaving the unreacted loaded RFID-oligo complexes in solution. In such an embodiment, RFID scanning can be performed while the reacted loaded RFID-oligo complexes are bound to the immobilized binding partners to determine which RFID tags are present in solution, which serves as a first indication as to the presence of the target agent in the sample. The capture-associated oligos may then be released into solution, or may be amplified by methods known in the art to provide a solution comprising oligos complementary to the electrode-associated oligos. This solution is subsequently exposed to the electrode-associated oligos, and hybridization detected by electrochemical means is a second indication that the target agent is present in the sample.

Certain embodiments of the present invention are particularly useful for screening molecules that are of pharmaceutical, therapeutic, diagnostic, prognostic, theranostic, neutriceutical, and/or other general biological interest or potential, including, inter alia, certain chemical moieties having potentially bioactive structures and/or conformations. In such embodiments, a moiety of interest or its associative partner (for example, a receptor, binding partner, associative complex or the like) is associated with an RFID tag. Such association can be, inter alia, direct or indirect. The means for attachment can be covalent, non-covalent, conjugated, derivatized or other interaction so as to provide a means to ensure an associative relationship with the RFID tag., Such relationship can be accomplished in one or more steps, before and/or after interaction of the moiety of interest and intended partner.

In accordance with some embodiments of the present invention, RFID tags are associated (directly or indirectly) with one or more molecules having pharmaceutical interest/potential (“drug”). The drug-derivatized RFID tags are contacted with tissue cultured cells containing or suspected of containing potential receptors for said drug, under conditions sufficient to allow the drug to interact with the receptor(s). The interaction of the drug, and hence the RFID tags, can be studied to determine such useful data as, inter alia, binding parameters (including, inter alia, rates of association, binding, strength of binding, rates of dissociation, rates of metabolism, etc.), by screening techniques known to those in the art coupled with interrogating the subject cells to determine if the RFID is associated with the cells. In a preferred embodiment, a mixture of potential drug candidates is used to derivative the RFID tags, and contacted (either in a mixed format or serially) to determine relative efficacy and preferred properties. Such methods are particularly suited for determining which specific compound or compounds were reactive and such additional information as relative reactivity and the like. In another embodiment, specific molecules are used to derivatize the RFID tags and then contacted with the subject cells thereby enabling, for example, determination of which cellular structure(s) they interact with.

In accordance with another embodiment of the present invention, RFID tags are used for in vivo studies that include, inter alia, model binding systems, diagnostics, prognostics, and therapeutics. For example, in some embodiments of the invention, derivatized RFID tags such as those described in the preceding paragraph are administered by the desired mode of administration for the particular compound/study of interest. Such methods typically include, for example, oral, rectal, subcutaneous, intramuscular, enterally, parenterally, sublingual, intravenous, transmucosal, nasal, transcutaneous, buccal, intradermal, intrathecal, intraosseous, etc. administration. For example, a rat model system can be employed wherein RFID tags are derivatized with a class (e.g., one or preferably more than one candidate compound) of compounds potentially useful for treating kidney disorder. Once administered, the RFID-tags can be tracked within the subject animals, by interrogating the animals with a suitable RFID reader to determine if, for example, the drug makes it to the intended tissue target and/or what other organs it interacts with.

In other embodiments of the present invention, in vivo methods are provided for determining the presence of a target agent in an organism. For example, such methods include, in varying orders and combinations, a) administering to a patient, in a clinically-effective amount, a first complex comprising a tracking component (e.g., an RFID tag) capable of generating a signal and a binding moiety capable of associating with the target agent in vivo; b) scanning the patient with a reader capable of detecting the signal; and c) detecting the signal. In some such embodiments, the binding moiety is an antibody, antigen, protein, ligand, nucleic acid, receptor, toxin, immunoglobulin, metabolite, hormone, receptor binding agent, or a plurality or combination thereof. In specific embodiments, the binding moiety is capable of binding a cancer marker, genetic mutation, nucleic acid sequence, protein, metabolite, toxin, drug, pathogen, microorganism, virus, or a plurality or combination thereof. Methods can further include interrogating the tracking component with a reader capable of generating a response signal from the tracking component of sufficient energy to destroy a cell associated with the target agent. For example, sufficient energy can be equivalent to 0.25-10 Gy. The cell destroyed may be, e.g., a cancer cell, a microorganism, a pathogen, or a virally-infected cell. The biological effects of radiation of widely known and used by those of ordinary skill in the art, for example, in radiosurgical procedures.

Similarly, RFID tags can be derivatized with other ligands, moieties, pharmaceuticals, compounds having potential/possible diagnostic, therapeutic or other pharmaceutical activity, antibodies, nucleic acids, synthetic derivatives of the forgoing, analogues, homologues, etc. In some embodiments, an RFID tag may be derivatized with a therapeutic molecule (including therapeutic and abused drugs, antibiotics, etc.); a naturally occurring molecule with known physiological function (including hormones, cytokines, proteins, lipids, carbohydrates, cellular membrane antigens and receptors (neural, hormonal, nutrient, and cell surface receptors) or their ligands, etc). Suitable ligands and receptors that may be derivatized to an RFID tag include an antibody or fragment thereof to be recognized by a corresponding antigen or epitope, a hormone to be recognized by its receptor, an inhibitor to be recognized by its enzyme, a co-factor portion to be recognized by a co-factor enzyme binding site, a binding ligand to be recognized by its substrate, and the like. For example, in a certain embodiments, a subject (human or animal) is treated, by appropriate administration, with antibody derivatized RFID tags. Depending on the purpose of the test or treatment, one or more antibodies (and/or other molecules) can be employed. In a representative, non-limiting embodiment, RFID tags are coated with monoclonal antibodies raised against and specific for the HER-2 breast cancer antigen(s). Following administration, the patient is subjected to an RFID interrogation protocol. The patient can be subject to a whole body scan to determine where, if at all, the antibody-derivatized RFID tags become bound—i.e., where in the body they interact. The means for scanning is dependent upon the RFID tags used (based on the frequency requirements as known to those skilled in the art). In certain embodiments, the RFID tags are selected so as to be interrogated by standard MRI equipment. In some embodiments, the interrogation is first performed at a low field strength (or series of successive scans at increasing strengths), thereby enabling the detection of any associated RFID tag. Where it is determined that the RFID tags are in fact associated, the field strength can be increased to a level where the strength of the signal response from the RFID tag is sufficient to kill or disable the cells in close proximity to the associated RFID tag. For example, in an example employing the HER-2 antibodies, following the confirmation of RFID tags to cancerous cells, the RFID tags are interrogated with a signal field strength sufficient to cause a responsive signal with enough energy to destroy or disable cells, thereby destroying or disabling those cells in close proximity to the associated RFID tags and, thereby, therapeutically treating the malignancy.

In preferred embodiments, an RFID tag is read by a reader device such as an electrochemical detector device or an optical scanner, many of which are known in the art. A reader device (e.g., an RFID reader) is placed within close proximity of an RFID device. Upon placement of the RFID device into the reader device, the reader device will interrogate the RFID device, collecting all relevant encoded information where such information may be used to pre-populate the corresponding fields of, for example, a data printout. Thus, manual data entry is eliminated and the risk of user error is reduced and/or eliminated. This is described in more detail in the co-pending application U.S. Ser. No. 60/809,578, filed May 31, 2006, entitled “Device and Methods for Tracking Diagnostic Samples and Results,” which is incorporated herein by reference. In some embodiments, the existence of an RFID tag is what is detected by the RFID reader, and not necessarily information stored on the RFID tag. In some embodiments, the detection of the presence of an RFID tag indicates the presence of one or more target agents.

A basic RFID system includes two components: an interrogator or reader and a transponder (commonly called an RF tag, herein also referred to as an RFID tag). The interrogator and RF tag may include respective antennas. In operation, the interrogator transmits through its antenna a radio frequency interrogation signal to the antenna of the RF tag. In response to receiving the interrogation signal, the RF tag produces an amplitude-modulated response signal that is transmitted back to the interrogator through the tag antenna by a process known as backscatter. In some embodiments, the RF tag does not comprise an antenna, and interrogation of the RF tag may require contact between or close proximity of the interrogator and the RF tag.

The conventional RF tag includes an amplitude modulator with a switch, such as a MOS transistor, connected between the tag antenna and ground. When the RF tag is activated by the interrogation signal, a driver creates a modulating on/off signal based on an information code, typically an identification code, stored in a non-volatile memory of the RF tag. The modulating signal is applied to a control terminal of the switch, which causes the switch to alternately open and close. When the switch is open, the tag antenna reflects a portion of the interrogation signal back to the interrogator as a portion of the response signal. When the switch is closed, the interrogation signal travels through the switch to ground, without being reflected, thereby creating a null portion of the response signal. In other words, the interrogation signal is amplitude-modulated to produce the response signal by alternately reflecting and absorbing the interrogation signal according to the modulating signal, which is characteristic of the stored information code. The RF tag could also be modified so that the interrogation signal is reflected when the switch is closed and absorbed when the switch is open. Upon receiving the response signal, the interrogator demodulates the response signal to decode the information code represented by the response signal. The conventional RFID systems thus operate on a single frequency oscillator in which the RF tag modulates a RF carrier frequency to provide an indication to the interrogator that the RF tag is present.

The substantial advantage of RFID systems is the non-contact, non-line-of-sight capability of the technology. The interrogator emits the interrogation signal with a range from one inch to one hundred feet or more, depending upon its power output and the radio frequency used. In some embodiments, the RFID tag located on an RFID device (e.g., a diagnostic device) when in use is proximally located to the interrogator so that the size of the antenna on the tag can be small, or the antenna on the tag can be eliminated altogether. The interrogator is located on the reader device in many embodiments, or otherwise located nearby in the laboratory facility. Preferably, the RFID operates at a low frequency such as 125 to 134 kHz or a high frequency such as 13.56 MHz. In other embodiments, the RFID operates at an ultra-high frequency such as 868 to 928 MHz. An overview of the RFID technology and its application is found in “RFID, Radio Frequency Identification,” Steven Shepard (McGraw-Hill Publishing 2005), the text of which is incorporated by reference. A general RFID reader may be capable of reading signals from a 4π region of space surrounding the detector. In many applications, including certain preferred embodiments of the present invention, such broad range detection may not be desirable due to the possibility of inaccurate readings cause by RF tags other than that which is desired. In a preferred embodiment, electromagnetic frequency (EMF) shields are employed to ensure that the reader only receives signals originating from the assay device in question.

In some embodiments of the present invention, the reader is configured such that one tag at a time passes through a path of travel and the tags are read one at a time. For instance, the sample could be passed through a tube where the RFID identification numbers are read at a certain portion of the tube, the rest of the sample being shielded from RFID interrogation. Alternatively, the sample could be placed in an hourglass shaped container. The constricted middle portion of the container is interrogated for RFID identification numbers, and allows the RFID tags to flow through the constricted section where they are read one by one. The sample is placed in the top portion of the hourglass, and flows into the bottom portion of the hourglass, said top and bottom portion being shielded from RFID interrogation. In another configuration, the RFID reader (or a portion thereof) can have immobilized binding partners affixed to or otherwise attached to the reader for immobilization of reacted RFID tags. The RFID reader can be in any shape such that a surface of the RFID reader can contact or be in close proximity to the reaction. The immobilized binding partners on the surface of the reader are designed to bind to the target agent, or anything associated with the target agent such as an antibody, antigen, nucleic acid, RFID tag, molecule or particle. With only reacted RFID tags associated with the target agent, only reacted RFID tags are immobilized at or near the RFID reader surface. RFID tags not immobilized near the surface may be washed or rinsed, or the reader's surface may be moved away or shielded from RFID tags not immobilized at or near the reader surface. In this manner, only RFID tags immobilized at or near the reader surface are interrogated. In some configurations, RFID tags are interrogated one by one by a reader, in other configurations, reacted and unreacted RFID tags are interrogated as individual groups. In some configurations, RFID tags are interrogated at intermediate steps throughout a reaction in addition to, or instead of, interrogating the RFID tags at the conclusion of the reaction.

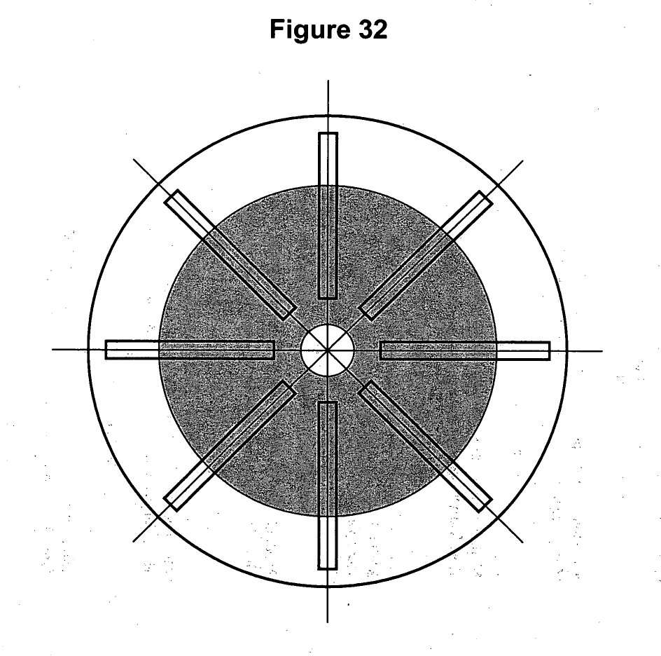

FIG. 32 illustrates an embodiment of a device for separating RFID devices such as those shown in FIG. 5 or other RFID devices where reacted loaded RFID complexes can be separated from unreacted loaded RFID complexes by centrifugation. FIG. 32 shows an 8-sample centrifugal tag separator. Reaction mixtures are introduced into the inner ends of the channels. During centrifugation, reacted and unreacted RFID complexes are separated. In some embodiments, the reacted loaded RFID complexes move to the outside end of the channels during centrifugation, and the RFID tags at the outside end of the channels are subsequently read. In other embodiments, the unreacted loaded RFID complexes move to the outside end of the channels during centrifugation, and the RFID tags at the inside end of the channels (i.e., those in reacted loaded RFID complexes) are subsequently read. Optionally, the channels may be RF-shielded except where the reacted loaded RFID complexes are known to be located subsequent to centrifugation. For example, FIG. 32 shows an embodiment in which the channels are RF-shielded at the outside end (see shading). In certain embodiments, the RF-shielding may comprise an RF-shielded enclosure, e.g., a printed metal Faraday cage into which unreacted loaded RFID complexes are migrated and/or held. In some embodiments, the RF-shielding could be part of the RFID reader.

In some embodiments of the present invention, the reader is configured such that multiple RF tags will be read in close temporal proximity to each other only if they are bound near to each other on a nucleic acid sequence. Such device may consist of drawing the sample through a thin tube, with a small portion of the tube being interrogated at a given time.

A typical RF tag system often contains a number of RF tags and the interrogator. RF tags are divided into three main categories. These categories are beam-powered passive tags, battery-powered semi-passive tags, and active tags. Each operates in fundamentally different ways.

The beam-powered RF tag is often referred to as a passive device because it derives the energy needed for its operation from the interrogation signal beamed at it. The tag rectifies the field and changes the reflective characteristics of the tag itself, creating a change in reflectivity that is seen at the interrogator. A battery-powered semi-passive RF tag operates in a similar fashion, modulating its RF cross-section in order to reflect a delta to the interrogator to develop a communication link. Here, the battery is the source of the tag's operational power. Finally, in the active RF tag, a transmitter is used to create its own radio frequency energy powered by the battery.

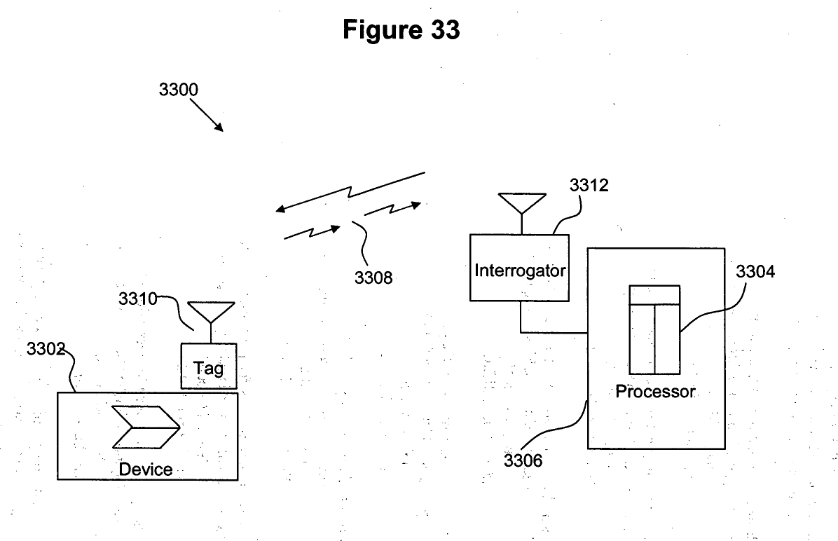

In some embodiments of the present invention, the system consists of three parts, a consumable hardware device, inventory and management software, and an interface between the hardware device and the software. FIG. 33 shows one embodiment of a system (3300) including a device (3302) (e.g., an RFID device), a management software component (3304) preferably implemented in a computer system (3306), and an interface (3308) coupling the device (3302) and the software (3304). Preferably, the interface (3308) includes a transponder (3310) associated with the device (3302) and an interrogator (3312), which is preferably located on the reader device and coupled to the computer-implemented system.

In specific embodiments, the transponder (3310) is associated with the device (3302), such as by affixing the transponder (3310) to an interior or exterior surface of the cartridge of the device (3302) or to an associated matrix. Alternatively, a transponder (3310) may be built into a device—particularly feasible when the device comprises an electronic detection array fashioned through photolithography or other semiconductor manufacturing means. Association of the transponder with the device can be achieved either during production of the device (3302) such that the transponder is embedded in the actual substrate of the device or after the device has been produced, such as through affixation of the transponder to a matrix.

The transponder (3310) can be preprogrammed with data about the device (3302), particularly a unique identifier, and other information depending on the size of the device, including but not limited to ownership information, location information, analysis information, production processes, clinical trial conduct, synthesis processes, sample collections, and other information known to those skilled in the art and that would be of value in managing samples. In addition to preprogramming such data, the transponder (3310) may be configured to permit modification and updating of the data within its memory. In addition, the transponder (3310) may contain security architecture that defines precise access conditions per type of data to thereby restricting reading, writing, and updating. For example, the interface (3308) components can be configured to receive control signals from and to respond to a particular computer-implemented data processing system, such as the software application described below. In addition, data written to the transponder (3310) can be encrypted for authentication and security purposes.

The use of RFID transponders or chips offers the benefit of a wide temperature range (typically −25° C. to +85° C.) without the loss of functionality. In addition, the RFID transponders can be utilized to control remote devices, such as a signaling light or generator of audible tones for alerting and locating the object associated with the transponder. Storage of information in the transponder (3310) also provides an additional backup should data in the computer-implemented system (3306) be damaged or lost.

The interrogator (3312) may be, e.g., a conventional radio frequency identification reader that is coupled to the computer-implemented system. Command and control signals are generated by the computer system (3306) to initiate interrogation of one or more transponders (3310) and to receive a response that is processed by the software (3304) in the computer-implemented system (3306). In one configuration, the transponders can be reprogrammed via communications from the interrogator to replace or update data stored therein.

In some implementations, one or more interrogators (3312) are positioned within a reader device or otherwise in a facility at a sufficient range to communicate via radio frequency signals, such as microwave signals, with the transponders (3310). Multiple interrogators can be used for multiple classes of transponders or with individual transponders. Alternatively, one interrogator utilizing known technology can communicate with multiple transponders on multiple frequencies in serial fashion or concurrently. In applications where multiple devices are processed, multiple interrogators positioned at various locations within a reader device or along a path of travel, such as a conveyor or sorting tube or apparatus can be used to track the location and the status of the device. Thus, the interface (3308) can be expanded to monitor and process data related to the movement and analysis of a device (3302) during biological production processes, or front end biological processing steps, washing steps and final analysis.

In many embodiments, the transponder is a passive device that is activated by the interrogation signal, from which it draws operating power. When the transponder is used to activate a remote device or to increase the range of communication, the transponder can be semi-active as described above. Alternatively, an active transponder can be used when large amounts of data are to be read from or written to the transponder or increased range is desired. Range is also affected by frequency, as is known in the art, and one of ordinary skill would select the appropriate frequency range in accordance with the environment, and the functional objectives. For example, certain specimens may be sensitive to particular frequencies of radio signals, and such frequencies would need to be avoided or the specimen appropriately shielded when designing the system.

Software (3304) is tailored for use with wireless communication systems and the processing of data associated with the life sciences. Such software consists of a customized user interface and a set of predefined database tables in one embodiment. A user can enter sample-associated data or import information from outside sources. Predefined tables may be provided in the database to facilitate setup of the system, but a user can have the option to customize fields within the tables. The relational database can include tables for chemical compounds, proteins, metabolites, lipids, cellular fractions, biological samples from different organisms such as viruses, bacteria, or multi-cellular organisms, or patient samples such as blood, urine, and buccal swabs. Detailed sample information and sample-associated data may be programmed into the tables. Clinical patient information can be, for example, age, gender, location, ethnic group, body mass index, family history, medication, data of onset of symptoms, duration of disease, and medical tests. Sample-associated data can consist of research data from various sources, such as, for example, protein data from Western blotting, ELISA or in-situ hybridization, bioassay data, drug screening data, and the like. Data can be supplied in the form of text, numbers, tables, or images. The software'(3304) may also link to other data sources and integrate information from public domains, such as GenBank, SwissProt, and other similar domains or proprietary or custom sources.

Optionally, the software (3304) is able to interface with robotics equipment to track the device within a process, and tracking of the process can be displayed as an accumulative device history for storage within the device as well as the database, such as storage in a transponder (3310), e.g., an RFID transponder.

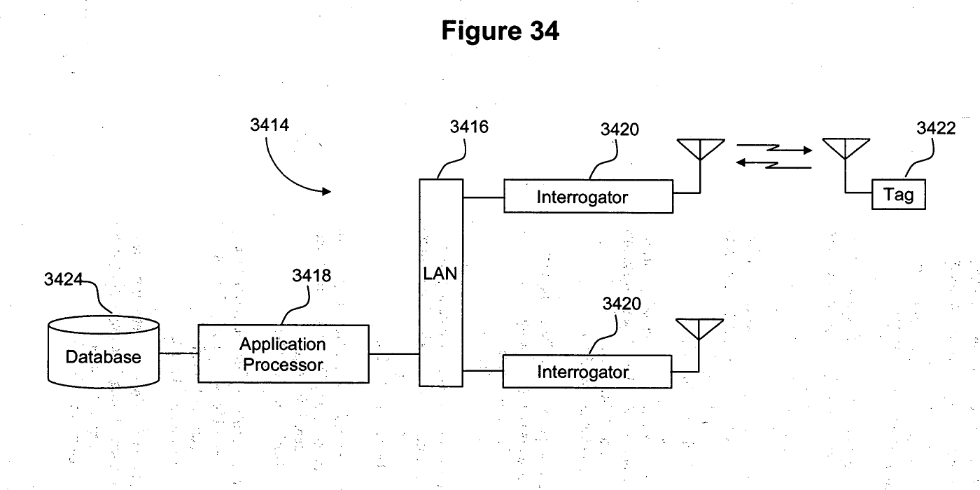

Shown in FIG. 34 is a computer-implemented system architecture (3414) for utilizing a local area network (3416) to interface an application processor (3418) with one or more interrogators (3420) that communicate with one or more remote tags (3422), e.g., RFID tags. The application processor (3418) is coupled to a database (3424). It is to be understood that the local area network can instead be a global network, such as the Internet, in which case web-based applications would be utilized.

The software (3304) is designed to create an informatics infrastructure where a single user may generate a data and information set, which is initially stored at a local workstation in a local database format. However, the software preferably is capable of linking multiple users in a hierarchical environment. The information accumulated by a single user can best be up-loaded to a centralized database system on a server. The interaction of the network environment can also be a web browser interface. The multi-user environment can be expanded to multiple-site environments, and software and databases can be located on a personal computer, on a server within an intranet or on the internet such as an e-commerce site. Access control and log control systems are also provided in the software. The application processor of the present invention may be coupled to a database; however, it is to be understood that the local area network can instead be a global network, such as the Internet, in which case web-based applications would be utilized, and data can be accessed transmitted or communicated by any means including, inter alia, internet, intranet, extranet, WAN, LAN, satellite communication, cellular phone communications, communications on a motherboard, and the like.

In some embodiments, the software has three components, a front end software component, a middleware component, and a back end software component. It is envisioned that the front end software is utilized to create a “user interface.” This can be, for example, a web browser, Microsoft Excel or a similar grid component. The web browser software would be used for a web-based system, whereas the Microsoft Excel software would be used for a desktop system. The web-based option provides for multiple users, networking, and can be expanded to accommodate thousands of users. The desktop option is sufficient for a single user who does not anticipate sharing of data and sample information via a network.

The middleware can include Microsoft Excel macros or grid components developed for use as a desktop option or custom software created by programming language suitable for use with web-based systems, such as PHP. The middleware is configured as a collection of programs that is capable of receiving user inputs and queries and returning database information to the user via known output, such as printer, display, or audible output.

The back end software can include Microsoft Access, which is proprietary database software offered by Microsoft Corporation and hosted by Microsoft Excel. This particular program provides sufficient database capacity to support up to 50,000 records, and to a maximum of 100,000 records with increasing levels of performance degradation. Another option is MySQL, which is a freeware database software developed collaboratively and available at no charge that runs on all major servers, including those based on Windows and Linux platforms. This database is capable of handling millions of records, and would be suitable for the large institutional user, such as governmental agencies, universities, and multinational entities.

The software (3304) is configured to provide control signals to the interface (3308) and to receive data and information from the interface (3308). In addition, when information is supplied to a transponder, the software is configured to initiate writing of the data through the interrogator (3312) to the transponder (3310) using methods and equipment known in the art and which is commercially available.

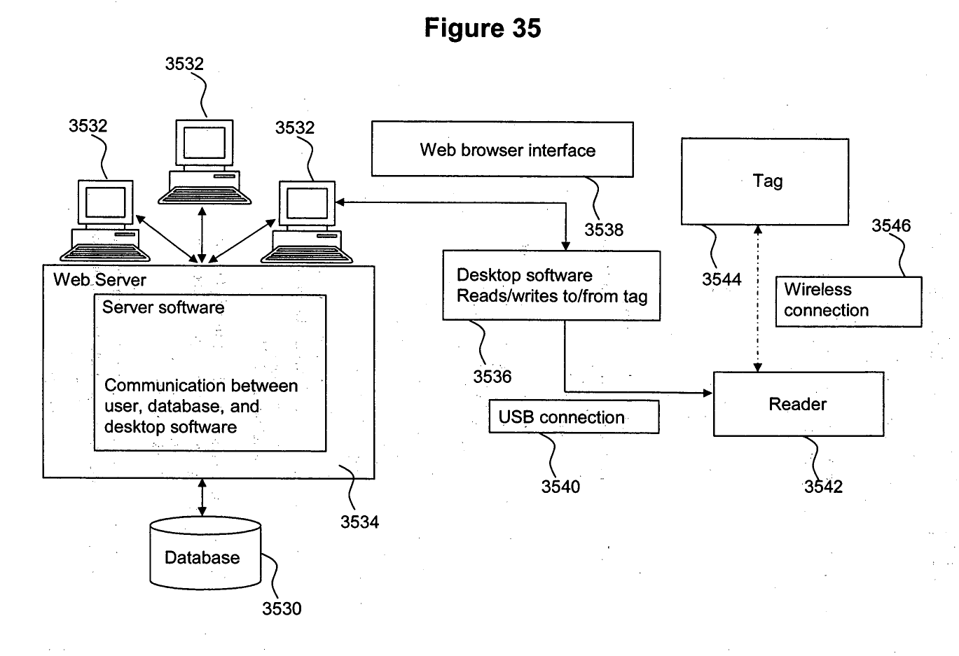

FIG. 35 illustrates another system architecture (3500) in which a database (3530) is linked to a plurality of desktop computers (3532) via a web server (3534). Resident on the server (3534) is software that provides a communication layer between the user, the database (3530), and desktop software (3536) resident on the desktop computers (3532). With a web browser interface (3538), a user can connect to the reader (3542) (e.g., RFID reader) through a standard USB connection 3540. The user can then control read and write operations of the reader (3542) and the remote tag (3544) (e.g., RFID tag) using the wireless connection (3546) provided by the radio frequency communications.

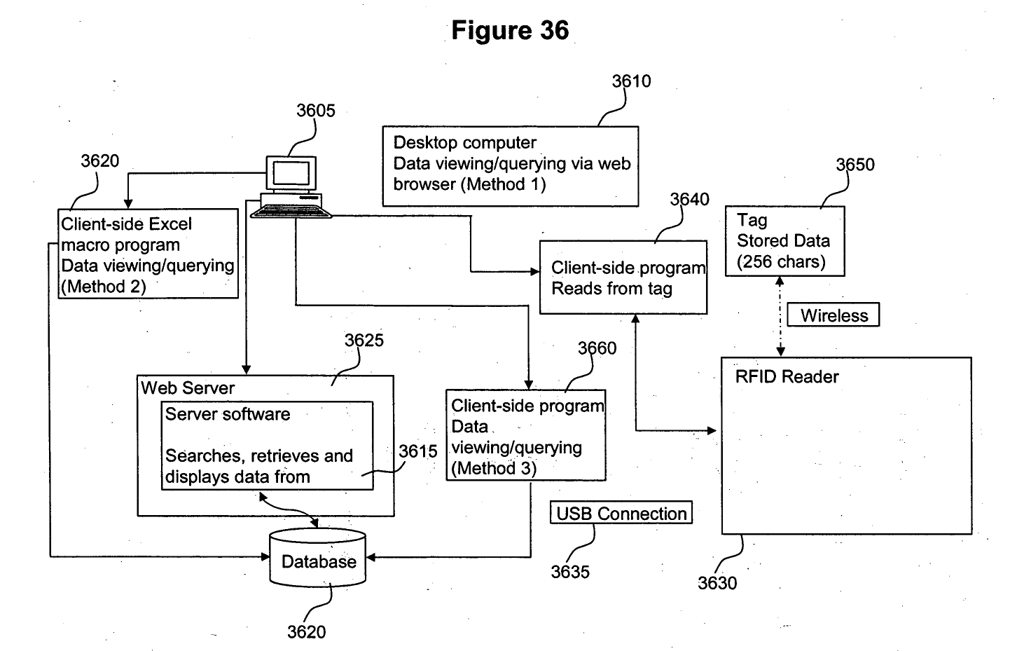

FIG. 36 shows another embodiment of the invention utilizing a 3-tier architecture (3600) having a desktop computer (3605) with a front-end web browser (3610) linked to a backend database (3620) via web server middleware (3615) on a web server (3625). The middleware provides search, retrieval, and display ability to a user. More particularly, the business logic is contained in the middleware program (3615) on the web server (3625). In addition, there is (optionally) a reader (3630) (e.g., RFID reader) coupled via a USB connection (3635) to the client-side program (3640) on the desktop computer (3605). The client-side application, which reads and writes to the tag (3650) (e.g., RFID tag) via the reader (3630), is launched from the web browser (3610).

In an alternative 2-tier arrangement of architecture (3600), there is an Excel front-end program (3660) on the desktop computer (3605) that communicates directly with the database (3620) at the back end. The business logic here is embodied in the Excel macro program. This method is particularly efficient for loading data (e.g., 96 rows of data corresponding to each well in a plate) into a database to take advantage of the Excel functions, such as copying, dragging down, etc.

In yet a further 2-tier arrangement of the architecture (3600), a stand-alone client application (3670) at the front end communicates directly with the database (3620) at the back end. The business logic is contained within the stand-alone client application, and a module for reading from and writing to the tag (3650) may also be contained within this application (3670). Here the advantage is that the application is compiled (the source code is not visible) and does not require third-party software (Excel, web-server). The drawback is that it is not as network compatible as the 3-tier architecture described above.

### Example I (Prophetic)

### Preparation of Monoclonal Antibodies

A peptide corresponding to amino acid residues in a desired antigen is synthesized with a peptide synthesizer (Applied Biosystems) according to methods known in the art. The peptide emulsified with Freund's complete adjuvant is used as an immunogen and administered to mice by footpad injection for primary immunization (day 0). The booster immunization is performed four times or more in total. The final immunization is carried out by the same procedure two days before the collection of lymph node cells. The lymph node cells collected from each immunized mouse and mouse myeloma cells are mixed at a ratio of 5:1. Hybridomas are prepared by cell fusion using polyethylene glycol 4000 or polyethylene glycol 1500 (GIBCO) as a fusing agent. The lymph node cells of the mouse are fused with mouse myeloma PAI cells (JCR No. B0113; Res. Disclosure Vol. 217, p. 155, 1982), and the resulting hybridomas are selected by culturing the fused cells in an ASF104 medium (Ajinomoto Co. Inc.) containing HAT supplemented with 10% fetal calf serum (FCS) and aminopterin. The reactivity of the culture supernatant of each hybridoma clone is measured by ELISA.

Screening by ELISA is performed by adding the immunogen into each well of a 96-well ELISA microplate (Corning Costar Co.). The plate is incubated at room temperature for 2 hours for the adsorption of the immunogen onto the microplate. The supernatants are discarded and then the blocking reagent (200 μl; phosphate buffer containing 3% BSA) is added into each well. The plate is incubated at room temperature for 2 hours to block free sites on the microplate. Each well is washed three times with 200 μl of phosphate buffer containing 0.1% Tween 20. Supernatant (100 μl) from each hybridoma culture is added into each well of the plate, and the reaction is allowed to proceed for 40 minutes. Each well is then washed three times with 200 μl of phosphate buffer containing 0.1% Tween 20. In the next step, biotin-labeled sheep anti-mouse immunoglobulin antibody (50 μl; Amersham) is added to the wells and the plates are incubated at room temperature for 1 hour.

The microplate is washed with phosphate buffer containing 0.1% Tween 20. A solution of streptavidin-β-galactosidase (50 μl; Gibco-BRL), diluted 1000 times with a solution (pH 7.0) containing 20 mM HEPES, 0.5 M NaCl and bovine serum albumin (BSA, 1 mg/mL), is added into each well. The plate is then incubated at room temperature for 30 minutes. The microplate is then washed with phosphate buffer containing 0.1% Tween 20. A solution of 1% 4-Methyl-umbelliferyl-β-D-galactoside (50 μl; Sigma) in a phosphate buffer (pH 7.0) containing 100 mM NaCl, 1 mM MgCl₂ and 1 mg/mL BSA, is added into each well. The plate is incubated at room temperature for 10 minutes. 1 M Na₂CO₃ (100 μl) is added into each well to stop the reaction. Fluorescence intensity is measured in a Fluoroscan II Microplate Fluorometer (Flow Laboratories Inc.) at a wavelength of 460 nm (excitation wavelength: 355 nm).

### Example II (Prophetic)

### Preparation of Magnetic Beads with Antibodies Immobilized on the Bead Surface

Magnetic particles (“beads”) may be used as a substrate upon which antibodies may be attached to form immobilized binding partners. The use of magnetic beads is well known in the art and these reagents are commercially available from such sources as Ademtech Inc., (New York, N.Y.) and Promega U.S. (Madison, Wis.). “Amino-Adembeads” are obtained from Ademtech and consist of a magnetic core encapsulated by a hydrophilic polymer shell, along with a surface activated with amine functionality to assist with immobilization of antibodies to the bead surface. The beads are first washed by placing the beads in the included “Amino 1 Activation Buffer,” then placing this reaction tube in a magnetic device designed for separation. The supernatant is removed, the reaction tube is removed from the magnet, and the beads are resuspended in the included “Amino 1 Activation Buffer.” To assist coupling of the antibody with the magnetic bead, EDC (1-ethyl-3-(3-dimethlaminopropyl) carbodiimide hydrochloride) (4 mg/mL) is dissolved into the included “Amino 1 Activation Buffer,” and an appropriate amount of this solution is added to the beads (80 μl/mg beads) and vortexed gently. 10-50 μg of antibodies is added per milligram of beads, and the solution is vortexed gently. The solution is incubated for 1 to 2 hours at 37° C. under shaking conditions. Bovine serum albumin (BSA) is then dissolved in “Amino 1 Activation Buffer” to a final concentration of 0.5 mg/mL, and 100 μl of this BSA solution is added to 1 mg of antibody-coated beads, and the solution is vortexed gently and incubated for 30 minutes at 37° C. under shaking. The beads are then washed in the included “Storage Buffer” twice, and the beads are resuspended.

### Example III (Prophetic)

### Preparation of Loaded-RFID Complexes

An RFID tag is first coated with parylene C using a parylene coating machine (Speedline Technologies). Optodex™ is immobilized to the parylene photochemically. The parylene/Optodex™-coated RFID tags are dried for 3 hours in a vacuum and the surfaces are photobonded by irradiation for 4 minutes in a UV crosslinker (Stratagene). The surfaces are rinsed with PBS containing 0.05% (vol/vol) Tween 20, PBS, and bidistilled water. The rinsing steps are repeated three times and include occasional shaking for 5 minutes. The parylene/Optodex™-coated RFID tags are treated for 16 hours with a 2 mg/mL solution of glutaric anhydride in dimethyl formamide. The surfaces are treated for 10 minutes with a solution of 0.05 M N-hydroxysuccinimide and 0.2 M N-(3-dimethylaminopropyl)-N-ethylcarbodiimide hydrochloride (EDC) in bidistilled water and rinsed for 5 minutes with PBS. The surfaces are incubated for 20 minutes in a solution containing 0.01 mg/mL of the antibody or antigen in a buffer such as acetate buffer, and rinsed for 5 minutes in PBS. The surfaces finally are treated for 10 minutes with 1 M ethanolamine solution, pH 8, and rinsed with buffer.

### Example IV (Prophetic)

### Preparation of RFID-Oligo Complexes

An RFID tag is first coated with parylene C using a parylene coating machine (Speedline Technologies). Optodex™ is immobilized to the parylene photochemically. The parylene/Optodex™-coated RFID tags are dried for 3 hours in a vacuum and the surfaces are photobonded by irradiation for 4 minutes in a UV crosslinker (Stratagene). The surfaces are rinsed with PBS containing 0.05% (vol/vol) Tween 20, PBS, and bidistilled water. The rinsing steps are repeated three times and include occasional shaking for 5 minutes. The parylene/Optodex™-coated RFID tags are treated for 16 hours with a 2 mg/mL solution of glutaric anhydride in dimethyl formamide. The surfaces are treated for 10 minutes with a solution of 0.05 M N-hydroxysuccinimide and 0.2 M N-(3-dimethylaminopropyl)-N-ethylcarbodiimide hydrochloride (EDC) in bidistilled water and rinsed for 5 minutes with PBS. The surfaces are incubated for 20 minutes in a solution containing 0.01 mg/mL of an oligonucleotide in a buffer such as acetate buffer, and rinsed for 5 minutes in PBS. The surfaces are finally treated for 10 minutes with 1 M ethanolamine solution, pH 8, and rinsed with buffer.

### Example V (Prophetic)

### Binding of Target Agent (E. coli O157:H7) and Removal of Excess Unreacted Loaded RFID

A sample is obtained from a patient suffering from an E. coli O157:H7 infection and then is diluted in PBS/Tween20. RFID tags are conjugated or otherwise associated with anti-E. coli O157:H7 antibodies to form loaded RFID complexes (a procedure for making such loaded RFID complexes is described, e.g., in Example III). The loaded RFID complexes are contacted with the diluted sample by adding a one-third volume of bovine serum albumin (12% [wt/vol] in PBS) and 2 μg of loaded RFID complexes. The mixture is incubated at room temperature for 60 minutes.

Unbound loaded RFID complexes are removed by magnetic microparticle depletion. Briefly, magnetic microparticles are coated with the epitope recognized by the anti-E. coli O157:H7 antibody. The epitope-coated magnetic beads are added to the reaction mixture, in a PBS buffer supplemented with 0.5% BSA and 2 mM EDTA, and incubated at 4° C. for 30 minutes. Only those loaded RFID complexes that have not bound to E. coli O157:H7 in the sample (unreacted loaded RFID complexes) are available to bind to the immobilized epitope. The magnetically-labeled unreacted loaded RFID complexes are separated from the reaction mixture by adding the mixture to a column packed with lattice-type matrix and applying a magnetic field. Such separation devices are known in the art (e.g., MACS® Columns, Miltenyi Biotec). The magnetically-labeled unreacted loaded RFID complexes are retained on the column, and the reacted loaded RFID complexes pass through the column and are available for detection.

### Example VI (Prophetic)

### Binding of Target Agent (E. coli O157:H7) and Alternative Method of Removal of Unreacted Loaded RFID

A sample is obtained from a patient suffering from an E. coli O157:H7 infection and is diluted in PBS/Tween20. RFID tags are conjugated to anti-E. coli O157:H7 antibodies to form loaded RFID complexes (a procedure for making such loaded RFID complexes is described, e.g., in Example III). The loaded RFID complexes are contacted with the diluted sample by adding a one-third volume of bovine serum albumin (12% [wt/vol] in PBS) and 2 μg of loaded RFID complexes. The mixture is incubated at room temperature for 60 minutes.

Unbound loaded RFID complexes are removed by magnetic microparticle depletion. Briefly, magnetic microparticles are coated with a second anti-E. coli O157:H7 antibody, specific to another region (epitope) of the same target agent to be detected (a procedure for making such antibody-coated magnetic microparticles is described, e.g., in Example II). The second antibody-coated magnetic beads are added to the reaction mixture, in a PBS buffer supplemented with 0.5% BSA and 2 mM EDTA, and incubated at 4° C. for 30 minutes. Only those loaded RFID complexes that have bound to E. coli O157:H7 in the sample (reacted loaded RFID complexes) bind to the magnetic particle-immobilized second anti-E. coli O157:H7 antibody. The magnetically-labeled reacted loaded RFID complexes are separated from the reaction mixture by adding the mixture to a column packed with lattice-type matrix and applying a magnetic field. Such separation devices are known in the art (e.g., MACS® Columns, Miltenyi Biotec). The magnetically-labeled reacted loaded RFID complexes are retained on the column. The unreacted loaded RFID complexes will pass through the column. The RFID tags in the reacted loaded RFID complexes may be scanned while they are bound to the column, or subsequent to removal from the column by methods known to those of skill in the art.

### Example VII (Prophetic)

### Alternative Method of Binding Target Agent (E. coli O157:H7) and Removal of Unreacted Loaded RFID

A sample is obtained from a patient suffering from an E. coli O157:H7 infection and is diluted in PBS/Tween20. Antibodies are covalently attached to magnetically-labeled microparticles utilizing techniques standard to those who practice the art (a procedure for making such magnetic microparticles coated with antibody is described, e.g., in Example II). Densities of antibodies on the magnetic microparticles are fairly standard such that one can expect that 7×10⁸ beads/mL typically results in approximately 10 mg/mL protein concentration. The magnetic microparticles are then washed two times with a solution comprising 10 mM phosphate buffered saline, pH 7.4 and 100 mM NaCl (PBSNa), and are resuspended in a minimal volume of PBSNa (approximately 100 μl) supplemented with BSA to final concentration of 2.75%. The sample suspected of containing the target agent (approximately 10 μl) is added into the mixture with the magnetic microparticles at the proportion of one tenth the volume of suspension containing the magnetic microparticles. The resultant mixture is incubated at room temperature with gentle shaking for 30-60 minutes. Preferably, one would anticipate that the binding partners immobilized on the surface of the magnetic particles are at concentrations that are in molar excess, preferably at least ten-fold molar excess, of the corresponding target agent present within the added sample mixture. The magnetically-labeled microparticle-target agent complex is separated from the reaction mixture by adding the mixture to a column packed with lattice-type matrix and applying a magnetic field. Such separation devices are known in the art (e.g., MACS® Columns, Miltenyi Biotec). The magnetically-labeled microparticle-target agent complex is retained on the column. The target agent that is not bound to the magnetically-labeled microparticle-target agent complex will pass through the column.

Loaded RFID complexes are generated with a second anti-E. coli O157:H7 antibody, (a procedure for making such loaded RFID complexes is described, e.g., in Example III) specific to another region (epitope) of the same target agent to be detected. The loaded RFID-antibody complexes (comprising the second anti-E. coli O157:H7 antibody) are added to the reaction mixture, in a PBS buffer supplemented with 0.5% BSA and 2 mM EDTA, and incubated at 4° C. for 30-60 minutes. Following incubation, a magnetic field is applied to separate the magnetically-labeled microparticle-target agent complex away from the remainder of the mixture. The magnetic microparticle-target agent complexes are washed twice with PBSNa and resuspended in 20 μl of PBSNa containing 3.75% BSA. Only those E. coli O157:H7 target agents that bound to magnetic particles in the first reaction are available to bind to the loaded RFID-antibody complexes in the second reaction. The magnetically-labeled loaded RFID-target agent complex is separated from the reaction mixture by adding the mixture to a column packed with lattice-type matrix and applying a magnetic field. The magnetically-labeled complex is retained on the column. The loaded RFID-antibody complexes that are not bound to the magnetically-labeled microparticle-target agent complex will pass through the column. the RFID tags in the loaded RFID complexes that are retained on the column are subjected to RFID interrogation, either on the column or subsequent to elution from the column.

### Example VIII (Prophetic)

### Binding of Target Agent (Human Anti-Hepatitis Antibodies) without Direct Interaction with the Causative Agent

A sample is obtained from a patient suspected of being infected with hepatitis. The sample is diluted in a diluent such as PBS/Tween20. Loaded RFID complexes with hepatitis-specific antigen are incubated with the diluted sample by adding a one-third volume of bovine serum albumin (12% [wt/vol] in PBS) and 2 μg of loaded RFID complexes. The resulting mixture is incubated at room temperature for 60 minutes.

Unreacted loaded RFID complexes are removed by magnetic microparticle-antibody affinity depletion. Briefly, magnetic micro-particles are coated with an antibody affinity reagent such as Protein A, Protein G or anti-class antibody that binds antibodies from the sample, some of which may be hepatitis antigen-specific and bound to the loaded RFID complexes to form reacted loaded RFID complexes. The coated magnetic beads are added to the reaction mixture, in a PBS buffer supplemented with 0.5% BSA and 2 mM EDTA, and incubated at 4° C. for 30 minutes. Reacted loaded RFID complexes will be immobilized on the magnetic beads, via the anti-hepatitis antibodies. The magnetic beads are extensively washed with PBS/Tween20. Reacted loaded RFID complexes are thereby separated from the rest of the sample. Such separation techniques are known in the art (e.g., MACS Columns, Miltenyi Biotec).

### Example IX (Prophetic)

### Multiplexed Detection of Binding Events Between Immobilized Enzyme Receptors and Enzyme Antagonists

The wells of a 96-well microtiter plate are filled with 0.05 mL of 0.1 M carbonate buffer, pH 9.6. Each enzyme receptor of interest is suspended in 0.1 M carbonate buffer, pH 9.6 to a final enzyme receptor concentration of about 10 μg/mL. 0.05 mL of each enzyme receptor solution is placed in a well containing the carbonate buffer solution, and the location of the placement of each enzyme receptor is noted. The 96-well plate is sealed and placed at 4° C. overnight. The enzyme receptor solution in the wells is discarded. The wells are then each filled with 0.2 mL dilution buffer (made from 0.5 g Tween20, 2.5 g bovine serum albumin, 1.0 g sodium azide, dissolved in up to 1 liter of phosphate buffered saline), and the plate is incubated for 1 hour at room temperature. The dilution buffer is discarded and refilled with 0.05 mL dilution buffer in each well. Loaded RFID complexes (the procedure for making such loaded RFID complexes in described, e.g., in Example II) containing enzyme agonists as capture moieties are placed in dilution buffer. Multiple different enzyme agonists can be tested against the enzyme receptors by noting the identification number of the RFID tag to which each enzyme is conjugated. 0.1 mL of the loaded RFID complexes in dilution buffer is placed in each well. The plate is sealed and incubated at 37° C. for 30 minutes. The solution in the plate is then discarded, and each well of the plate is individually interrogated by an RFID reader. Loaded RFID complexes that have bound to immobilized enzyme receptors will remain on the plate, and the identification numbers of these reacted loaded RFID complexes can be correlated to the location on the plate to which they bound, and hence to which enzyme receptor the loaded RFID complex bound.

### Example X (Prophetic)

### Hybridization of RFID-Oligo Complexes to Target Nucleic Acid Molecules

In embodiments utilizing RFID-oligo complexes for the detection of target nucleic acid or genotyping of single nucleotide polymorphisms in target nucleic acid, an RFID-oligo complex is hybridized to the target nucleic acid. The hybridization reaction is carried out as follows. RFID-oligo complexes (the procedure for making such RFID-oligo complexes is described, e.g., in Example IV) are mixed with target DNA in 2×SSC solution (300 mmol/L NaCl, 30 mmol/L trisodium citrate). The hybridization reaction is carried out for five minutes at a temperature that permits specific hybridization of the two nucleic acid molecules. The temperature of the hybridization reaction is determined using the equation for calculating the melting temperature of an oligonucleotide. Stringent hybridization conditions may be used such as a temperature about 5-10° C. lower than the melting temperature of the sequence, reduced salt concentration (typically 0.01 to 1.0 M), and pH of between 7.0 and 8.3.

It is to be understood that, in the foregoing various embodiments or methods described herein, modifications to steps or reaction components can be made by persons skilled in the art without departing from the spirit of the invention. For example, where a moiety such as an oligo, particle, reactive group, antibody, or binding partner and the like is described as being affixed to or otherwise attached to a particle such as a scaffold, RFID tag, magnetic bead, oligo and the like, a plurality of moieties may be attached, even where a particle is described or illustrated as having only one such moiety attached. Where a wash, rinse, or selection step is described, it is to be further understood that more than one wash, rinse, or selection step may be employed, and that such wash, rinse, or selection steps may occur at various points in the method. In some embodiments of the methods of the present invention, it may be advantageous to include several to many wash and selection steps. The specific wash and selection steps described herein for certain embodiments should not limit the methods of the present invention in any way. Finally, although the methods of the present invention describe a particular progression of steps, such steps can be performed in varied orders or combinations.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The abstract and the title are not to be construed as limiting the scope of the present invention, as their purpose is to enable the appropriate authorities, as well as the general public, to quickly determine the general nature of the invention. The scope of the invention will be measured by the appended claims along with the full scope of equivalents to which such claims are entitled. In the claims that follow, unless the term “means” is used, none of the features or elements recited therein should be construed as means-plus-function limitations pursuant to 35 U.S.C. §112, ¶6. All publications mentioned herein are cited for the purpose of describing and disclosing reagents, methodologies and concepts that may be used in connection with the present invention. Nothing herein is to be construed as an admission that these references are prior art in relation to the inventions described herein. Throughout the disclosure various patents, patent applications and publications are referenced. Unless otherwise indicated, each is incorporated by reference in its entirety for all purposes.

1. A method of determining a presence of a target agent in a sample comprising:
(a) mixing said sample with tracking complexes comprising a tracking component conjugated to a capture moiety specific for said target agent, thereby producing a first mixture comprising reacted complexes, which are those of said tracking complexes that are associated with said target agent, and unreacted complexes, which are those of said tracking complexes that are not associated with said target agent;
(b) separating said reacted complexes from said unreacted complexes by contacting said first mixture with immobilized binding partners, wherein said immobilized binding partners facilitate separation of said unreacted complexes from said reacted complexes to produce a second mixture comprising said unreacted complexes and a third mixture comprising said reacted complexes; and
(c) detecting a presence of said reacted complexes by detecting a signal of said tracking component in said third mixture, which is indicative of said presence of said target agent in said sample.

2. The method of claim 1, wherein said tracking component is an RFID tag.

3. The method of claim 2, wherein said detection device is an RFID detection device that transmits a radio frequency interrogation signal to said RFID tag, and further wherein in response to receiving said interrogation signal, said RFID tag produces a response signal.

4. The method of claim 1, wherein said detection device further comprises a matrix upon which said immobilized binding partners are attached.

5. The method of claim 1, further comprising an additional step of introducing said third mixture to said detection device prior to step (d).

6. The method of claim 1, wherein said tracking component is conjugated to a plurality of capture moieties.

7. The method of claim 1, wherein said tracking complex further comprises a polymer material upon which said capture moiety is conjugated.

8. The method of claim 7, wherein said polymer material comprises at least one member selected from the group consisting of: acrylics, vinyls, nylons, polyurethanes, polycarbonates, polyamides, polysulfones, polylactic acid, polyglycolic acid, polydimethylsiloxanes, polyetheretherketone, polytetrafluoroethylene, polyester, polyolefin, polyethylene terephthalate, polyethylene, polyether urethane, polysiloxane urethane, polyglycolic acid, and polyvinyl alcohol.

9. The method of claim 1, wherein said tracking complex further comprises an adaptor molecule.

10. The method of claim 9, wherein said adaptor molecule is avidin or streptavidin.

11. The method of claim 9, wherein said adaptor molecule is an antibody or an antigen.

12. The method of claim 1, wherein said tracking complex further comprises at least one oligonucleotide.

13. The method of claim 1, wherein said tracking complex further comprises at least one reactive group.

14. The method of claim 1, wherein said capture moiety is at least one member selected from the group consisting of antibodies, antigens, proteins, ligands, receptors, nucleic acids, toxins, immunoglobulins, metabolites, and hormones.

15. The method of claim 1, wherein said immobilized binding partners comprise a portion of said target agent, wherein said portion specifically reacts with said capture moieties.

16. The method of claim 1, wherein said immobilized binding partners specifically interact with said capture moiety when said capture moiety has not bound said target agent, thereby immobilizing unreacted complexes in an immobilized phase and leaving said reacted complexes in a solution phase.

17. The method of claim 1, further comprising:
(a) specific binding of said immobilized binding partners with said capture moieties when said capture moieties have bound said target agent, thereby immobilizing said reacted complexes in an immobilized phase and leaving said unreacted complexes in a solution phase, wherein said second mixture comprises said solution phase; and
(b) separating said second mixture from said immobilized phase, wherein said third mixture comprises said reacted complexes in said immobilized phase.

18. The method of claim 17, further comprising liberating said reacted complexes from said immobilized phase prior to said detecting said presence of said reacted complexes in step (c).

19. The method of claim 1, further comprising:
(a) specific binding of said immobilized binding partners with said target agent or a capture moiety/target agent complex, thereby immobilizing said reacted complexes in an immobilized phase and leaving said unreacted complexes in a solution phase, wherein said second mixture comprises said solution phase; and
(b) separating said second mixture from said immobilized phase, wherein said third mixture comprises said reacted complexes in said immobilized phase.

20. The method of claim 19, further comprising liberating said reacted complexes from said immobilized phase prior to said detecting said presence of said reacted complexes in step (c).

21. The method of claim 1, wherein said immobilized binding partners are immobilized on a matrix.

22. The method claim 21, wherein said matrix is composed of at least one particle.

23. The method of claim 22, wherein said particle is a bead.

24. The method of claim 23, wherein said bead is a magnetic bead.

25. The method of claim 24, further comprising a step of magnetically separating said reacted complexes from said unreacted complexes.

26. The method of claim 22, wherein said particle allows for isolation of said immobilized binding partners by at least one technique selected from the group consisting of centrifugation, size exclusion chromatography, affinity chromatography, ion exchange chromatography, HPLC, FPLC, magnetic capture, electrophoresis, dialysis, and filtration.

27. The method of claim 21, wherein said immobilized binding partners on said matrix specifically bind to said reacted complexes, and prior to step (c), at least said RFID component of said reacted complexes is released from said matrix.

28. The method of claim 21, wherein said matrix is a vessel or is contained within a vessel.

29. The method of claim 21, wherein said matrix is a column or is contained within a column.

30. The method of claim 21, wherein said matrix is a substrate upon which a plurality of immobilized binding partners are positioned at known locations, wherein immobilization of a first of said reacted complexes at a first location on said matrix is indicative that a first of said immobilized binding partners at said first location specifically associates with at least a portion of said first of said reacted complexes.

31. The method of claim 30, wherein said plurality of immobilized binding partners comprises distinct binding partners, wherein each of said distinct binding partners specifically interacts with a different reacted complex or a different portion of one of said reacted complexes than others of said distinct binding partners.

32. The method of claim 1, wherein said immobilized binding partners are selected from the group consisting of proteins, ligands, enzyme substrates, receptors, antigens, antibodies, toxins, immunoglobulins, metabolites, hormones, and nucleic acids.

33. The method of claim 1 wherein said target agent is an antibody and said immobilized binding partners are selected from the group consisting of protein A, protein G, a thiophilic resin, and an anti-class-specific antibody specific for a class of antibodies comprising said target agent.

34. The method of claim 1, wherein there are multiple capture moieties in said first mixture.

35. The method of claim 34, wherein
(a) said sample comprises one or more target agents;
(b) each of said multiple capture moieties is specific for a different one of said one or more target agents in said sample or is specific for a different portion of said one or more target agents in said sample;
(c) each of said multiple capture moieties is conjugated to a different tracking component, wherein each different tracking component comprises information to identify to which of said multiple capture moieties said different tracking component is conjugated;
(d) said third mixture comprises multiple reacted complexes; and
(e) said detection device detects each said different tracking component, thereby allowing simultaneous detection of said one or more target agents in said detection device.

36. The method of claim 35, wherein said one or more target agents include members selected from at least two of the classes consisting of proteins, ligands, receptors, nucleic acids, toxins, immunoglobulins, metabolites, and hormones.

37. The method of claim 35, wherein each said different tracking component has identification information to distinguish each said different tracking component from every other said different tracking component.

38. The method of claim 35, wherein each said different tracking component employs a different RFID frequency than every other said different tracking component.

39. The method of claim 1, wherein a first tracking component in one of said reacted complexes has at least one characteristic that is different from a second tracking component in one of said unreacted complexes, wherein said detection device can distinguish between said first tracking component and said second tracking component based on said at least one characteristic that is different.

40. The method of claim 1, wherein said detection device does not produce a signal indicating that said target agent is absent from said sample.

41. The method of claim 1, wherein said target agent is selected from the group consisting of organic and inorganic molecules, receptors, ligands, metabolites, steroids, hormones, lectins, sugars, proteins, enzymes, agonists, antagonists, antibodies, antigens, lipids, toxins, venoms, drugs, small molecules, nucleic acids, therapeutic molecules, cytokines, carbohydrates, whole cells, cell surface structures, viruses, spores, and portions and combinations thereof.

42. The method of claim 41, wherein said target agent is a nucleic acid comprising a SNP position, and said signal is indicative of a genotype at said SNP position.

43. The method of claim 42, further comprising processing said target agent in said sample prior to step (a), wherein said processing is selected from the group consisting of amplifying, fragmenting, labeling, denaturing, purifying, and cleaving.

44. The method of claim 42, further comprising at least two sets of tracking complexes, wherein a first set of tracking complexes comprises capture moieties perfectly complementary to a first genotype at said SNP position and a second set of tracking complexes comprises capture moieties perfectly complementary to a second genotype at said SNP position.

45. The method of claim 44, wherein a reader detects a signal based on a presence of said first set of tracking complexes, which indicates that said target agent comprises said first genotype at said SNP position.

46. The method of claim 44, wherein a reader detects a signal based on a presence of said second set of tracking complexes, which indicates that said target agent comprises said second genotype at said SNP position.

47. The method of claim 42, wherein said unreacted complexes in said second mixture are immobilized on said immobilized binding partners.

48. The method of claim 42, wherein said reacted complexes in said third mixture are immobilized on said immobilized binding partners.

49. The method of claim 1, wherein said target agent is a nucleic acid and said capture moiety is a first capture oligo, and further wherein said first mixture further comprises reactive group complexes comprising a reactive group conjugated to a second capture oligo, and further wherein said reacted complexes comprise those of said tracking complexes that are associated with said target agent further complexed with said reactive group complexes, and further wherein said immobilized binding partners specifically associate with said reactive group.

50. The method of claim 49, wherein said reacted complexes are subjected to a ligation and a denaturation prior to said contacting of step (b).

51. The method of claim 50, wherein said nucleic acid comprises a SNP position, and further wherein a first genotype at said SNP position promotes said ligation and a second genotype at said SNP position prevents said ligation.

52. The method of claim 50, wherein a polymerization is performed subsequent to said ligation and said denaturation and prior to said contacting of step (b).

53. The method of claim 1, wherein reactive groups are added to said reacted complexes and said immobilized binding partners specifically associate with said reactive groups to immobilize said reacted complexes.

54. The method of claim 1, wherein said tracking complexes' are a first set of tracking complexes, and further wherein said first mixture further comprises a second set of tracking complexes, wherein said first set associates with a different portion of said target agent than said second set, and further wherein a proximity of a one of said first set of tracking complexes and a one of said second set of tracking complexes is indicative of said target agent.

55. The method of claim 1, wherein said immobilized binding partners specifically associate with said reacted complexes at a position corresponding to that at which said tracking complexes are associated with said target agent.

56. The method of claim 55, wherein said target agent is a nucleic acid, said capture moiety is a nucleic acid, and said immobilized binding partners specifically associate with double-stranded nucleic acids.

57. The method of claim 56, wherein said immobilized binding partners are antibodies.

58. The method of claim 1, wherein said immobilized binding partners specifically associate with reactive groups, wherein said reactive groups are conjugated to moieties that specifically associate with said reacted complexes at a position corresponding to that at which said tracking complexes are associated with said target agent.

59. A method of determining a presence of a target agent in a sample comprising:
(a) introducing said sample to a matrix comprising one or more immobilized binding partners that specifically bind to said target agent, thereby facilitating formation of an immobilized complex comprising said target agent and one of said one or more immobilized binding partners;
(b) contacting said immobilized complex with tracking complexes comprising a tracking component conjugated to a capture moiety that interacts with said immobilized complex, thereby producing a mixture comprising reacted complexes, which are those of said tracking complexes that are associated with said immobilized complex, and unreacted complexes, which are those of said tracking complexes that are not associated with said immobilized complex, wherein said reacted complexes are in an immobilized phase and said unreacted complexes are in a solution phase;
(c) providing a detection device comprising a reader, wherein said reader detects a signal based on a presence of said tracking component;
(d) operating said detection device to interrogate said immobilized phase; and
(e) detecting said signal, wherein said signal is indicative of said presence of said tracking component in said immobilized phase, which is indicative of said presence of said target agent in said sample.

60. The method of claim 59, wherein said tracking component is an RFID tag.

61. The method of claim 59, wherein said matrix is a component of said detection device.

62. The method of claim 59, wherein said target agent and said one or more immobilized binding partners are nucleic acids.

63. The method of claim 62, wherein said matrix comprises immobilized binding partners that are perfectly complementary to at least a region of said target agent, and immobilized binding partners that are not perfectly complementary to said region of said target agent.

64. The method of claim 59, wherein one of said one or more immobilized binding partners binds to a first region of said target agent and said capture moiety binds to a second region of said target agent.

65. The method of claim 59, wherein said capture moiety does not interact with said target agent if said target agent has not bound to one of said one or more immobilized binding partners.

66. The method of claim 59, further comprising a wash step to remove said unreacted complexes prior to said detecting of step (e).

67. The method of claim 59, wherein step (a) further comprises adding a moiety complex comprising a first moiety that specifically interacts with said capture moiety and a second moiety that specifically interacts with said target agent prior to step (b), wherein said immobilized complex further comprises said moiety complex.

68. The method of claim 67, wherein said capture moiety is a reactive group and said second moiety is a nucleic acid.

69. The method of claim 59, further comprising determining a presence of plurality of target agents in a sample, wherein said one or more immobilized binding partners comprise a plurality of different immobilized binding partners, each of which specifically binds to only one of said plurality of target agents, and each of which is at a known location on a matrix; and further wherein said detecting further comprises determining a signal location on said matrix from which said signal is generated and correlating said signal location to which of said plurality of different immobilized binding partners is known to be at said signal location on said matrix, thereby identifying which of said plurality of target agents is in said sample.

70. A method of determining a presence of a target nucleic acid in a sample comprising:
(a) mixing said sample with a capture oligo to create a first mixture comprising hybridized complexes, which are those of said capture oligos that are associated with said target nucleic acid;
(b) adding tracking complexes to said first mixture, wherein each of said tracking complexes comprises a tracking component conjugated to a capture moiety specific for said hybridized complexes, thereby producing a second mixture comprising reacted complexes, which are those of said tracking complexes that are associated with said hybridized complexes, and unreacted complexes, which are those of said tracking complexes that are not associated with said hybridized complexes;
(c) isolating said reacted complexes from said unreacted complexes;
(d) providing a detection device comprising a reader, wherein said reader detects a signal based on a presence of said tracking component;
(e) operating said detection device to interrogate said reacted complexes subsequent to said isolating of step (c); and
(f) detecting said signal, wherein said signal is indicative of said presence of said target agent in said sample.

71. The method of claim 70, wherein said capture moiety is an antibody.

72. The method of claim 70, further comprising denaturing said target nucleic acid to facilitate formation of said hybridized complexes.

73. The method of claim 70, wherein said isolating of step (c) is performed using column chromatography.

74. The method of claim 73, wherein said column chromatography is selected from the group consisting of affinity chromatography, size-exclusion chromatography, and ion-exchange chromatography.

75. The method of claim 73, further comprising determining a presence of a plurality of target agents in a sample;
(a) wherein said capture oligo comprises a plurality of capture oligos, each of which specifically binds to only one of said plurality of target agents;
(b) wherein said capture moiety comprises a plurality of capture moieties, and said tracking component comprises a plurality of tracking components, and each of said plurality of capture moieties is conjugated to one of said plurality of tracking components that is specific therefor (that is, not conjugated to any other of said plurality of capture moieties);
(c) wherein each of said plurality of said capture moieties specifically binds to only one of said hybridized complexes, thereby creating a set of said reacted complexes, wherein each of said set links a given target agent of said plurality of target agents to a given tracking component of said plurality of tracking components such that no other of said reacted complexes comprises said given target agent with a different tracking component than said given tracking component, and no other of said reacted complexes comprises said given tracking component with a different target agent than said given target agent;
(d) further wherein said detecting further comprises determining from said signal which of said plurality of tracking components is in said set of reacted complexes, thereby identifying which of said plurality of target agents is in said sample.

76. The method of claim 70, wherein said isolating of step (c) is performed by contacting said second mixture to a matrix comprising immobilized binding partners, wherein said immobilized binding partners facilitate separation of said unreacted complexes from said reacted complexes to produce a third mixture comprising said unreacted complexes and a fourth mixture comprising said reacted complexes.

77. The method of claim 76, further comprising determining a presence of a plurality of target agents in a sample;
(a) wherein said capture oligo comprises a plurality of capture oligos, each of which specifically binds to only one of said plurality of target agents;
(b) wherein said capture moiety comprises a plurality of capture moieties, and said tracking component comprises a plurality of tracking components, and each of said plurality of capture moieties is conjugated to one of said plurality of tracking components that is specific therefor (that is, not conjugated to any other of said plurality of capture moieties);
(c) wherein each of said plurality of said capture moieties specifically binds to only one of said hybridized complexes, thereby creating a set of said reacted complexes, wherein each of said set links a given target agent of said plurality of target agents to a given tracking component of said plurality of tracking components such that no other of said reacted complexes comprises said given target agent with a different tracking component than said given tracking component, and no other of said reacted complexes comprises said given tracking component with a different target agent than said given target agent;
(d) further wherein said detecting further comprises determining from said signal which of said plurality of tracking components is in said set of reacted complexes, thereby identifying which of said plurality of target agents is in said sample.

78. The method of claim 76, wherein said immobilized binding partners immobilize said unreacted complexes on said matrix.

79. The method of claim 76, wherein said immobilized binding partners immobilize said reacted complexes on said matrix.

80. The method of claim 79, further comprising determining a presence of a plurality of target agents in a sample,
(a) wherein said capture oligo comprises a plurality of capture oligos, each of which specifically binds to only one of said plurality of target agents, thereby producing a plurality of hybridized complexes, each with a unique combination of one of said plurality of target agents and one of said plurality of capture oligos;
(b) wherein said immobilized binding partners comprise a set of immobilized binding partners, wherein each of said set is at a known location on said matrix, and further wherein each of said set specifically binds to only one of said plurality of hybridized complexes, thereby immobilizing a reacted complex comprising said one of said plurality of reacted complexes on said matrix; and
(c) wherein said detecting further comprises determining a signal location on said matrix from which said signal is generated and correlating said signal location to which of said set of immobilized binding partners is known to be at said signal location on said matrix, thereby identifying which of said plurality of hybridized complexes is bound at said signal location, and thereby determining which of said plurality of target agents is in said sample.

81. A method of determining a presence of a target agent in a sample comprising:
(a) labeling components of said sample including said target agent to create a first mixture comprising labeled target agent;
(b) mixing said first mixture with tracking complexes comprising a tracking component conjugated to a capture moiety that interacts with said labeled target agent, thereby producing a second mixture comprising reacted complexes, which are tracking complexes that are associated with said labeled target agent, and unreacted complexes, which are tracking complexes that are not associated with said labeled target agent;
(c) separating said unreacted complexes from said reacted complexes to produce a third mixture comprising said unreacted complexes and a fourth mixture comprising said reacted complexes;
(d) providing a detection device comprising a reader, wherein said reader detects a signal based on a presence of said tracking component;
(e) operating said detection device to interrogate said fourth mixture; and
(f) detecting said signal, wherein said signal is indicative of said presence of said tracking component in said fourth mixture, which is indicative of said presence of said target agent in said sample.

81. The method of claim 81, wherein said tracking component is an RFID tag.

82. The method of claim 81, wherein said separating of step (c) is performed by applying a magnetic field to said second mixture, wherein said magnetic field facilitates separation of said unreacted complexes from said reacted complexes to produce said third mixture comprising said unreacted complexes and said fourth mixture comprising said reacted complexes.

83. The method of claim 83, wherein said labeled target agent is labeled with a magnetic label.

84. The method of claim 83, wherein said labeled target agent is labeled with a reactive group, and further wherein said second mixture is contacted with immobilized binding partners specific for said reactive group, wherein said immobilized binding partners are conjugated to a magnetic label.

85. The method of claim 81, further comprising determining the presence of a plurality of target agents in said sample, wherein said capture moiety comprises a plurality of capture moieties, wherein each of said plurality of capture moieties is specific for one of said plurality of target agents or is specific for a different portion of said one or more target agents in said sample, and further wherein said signal is indicative of which of said plurality of target agents is in said sample.

86. A method of determining a presence of a target agent in a sample comprising:
(a) labeling components from said sample with tracking components to create a first mixture comprising a tracking component-target agent complex;
(b) contacting said first mixture with immobilized binding partners, wherein said immobilized binding partners facilitate immobilization of said tracking component-target agent complex to produce a second mixture comprising immobilized tracking component-target agent complex and a third mixture comprising any of said tracking components that did not form said tracking component-target agent complex;
(c) separating said second mixture from said third mixture;
(d) providing a detection device comprising a reader, wherein said reader detects a signal based on a presence of said tracking component;
(e) operating said detection device to interrogate said second mixture; and
(f) detecting said signal, wherein said signal is indicative of said presence of said one of said tracking components in said second mixture, which is indicative of said presence of said target agent in said sample.

88. The method of claim 87, wherein said tracking components are RFID tags.

89. The method of claim 87, wherein said immobilized binding partners are immobilized on a matrix that is a component of said detection device.

90. The method of claim 87, further comprising determining a presence of a plurality of target agents, wherein said immobilized binding partners comprise a set of immobilized binding partners, wherein each of said set is at a known location on a matrix, and further wherein each of said set specifically associates with only one of said plurality of target agents, and further wherein said detecting further comprises determining a signal location on said matrix from which said signal is generated and correlating said signal location to which of said set of immobilized binding partners is known to be at said signal location on said matrix, thereby identifying which of said plurality of target agents is in said sample.

91. A method of detecting binding between a target agent and a capture moiety, comprising:
(a) immobilizing said target agent to produce an immobilized target agent;
(b) introducing a tracking complex to said immobilized target agent, wherein said tracking complex comprises a tracking component and a capture moiety, wherein said capture moiety specifically interacts with said immobilized target agent, thereby producing
(c) an immobilized phase comprising reacted complexes, which are those of said tracking complexes that are associated with said immobilized target agent, and
(d) a solution phase comprising unreacted complexes, which are those of said tracking complexes that are not associated with said target agent;
(e) providing a detection device comprising a reader, wherein said reader detects a signal based on a presence of said tracking component;
(f) operating said detection device to interrogate said immobilized phase; and
(g) detecting said signal, wherein said signal is indicative of said presence of said tracking component in said immobilized phase, which is indicative of said binding between said target agent and said capture moiety.

92. The method of claim 91, further comprising separating said solution phase from said immobilized phase prior to said detecting of step (e).

93. The method of claim 91, wherein said tracking component is an RFID tag.

94. The method of claim 91, wherein said immobilized target agent is immobilized at a known location on a matrix.

95. The method of claim 91, wherein said target agent comprises a plurality of target agents immobilized at a plurality of known locations on a matrix, and wherein said capture moiety comprises a plurality of capture moieties, and wherein said tracking component comprises a plurality of tracking components, and further wherein each of said plurality of capture moieties (1) specifically interacts with a different one of said plurality of target agents and (2) is conjugated to a different one of said plurality of tracking components, and further wherein said detecting further comprises:
(a) determining at which of said plurality of known locations on said matrix is said signal generated, thereby identifying which of said plurality of target agents is in one of said reacted complexes, and
(b) determining which of said plurality of tracking components is generating said signal, thereby identifying which of said plurality of capture moieties is in said one of said reacted complexes.

96. The method of claim 94, wherein said matrix is a component of said detection device.

97. A composition comprising:
(a) a matrix;
(b) an immobilized binding partner associated with said matrix;
(c) a target agent associated with said immobilized binding partner; and
(d) a tracking component associated with said target agent.

98. The composition of claim 97, wherein said tracking component is an RFID tag.

99. The composition of claim 97, further comprising a reactive group.

100. The composition of claim 97, wherein said tracking component is further associated with a capture moiety.

101. The composition of claim 97, further comprising a detection device.

102. A system for determining a presence of a target agent in a sample comprising:
(a) a sample containing said target agent;
(b) tracking components associated with said target agent; and
(c) a detection device capable of detecting said tracking components associated with said target agent.

103. The system of claim 102, wherein said detection device comprises one or more immobilized binding partners.

104. A method of doing business wherein the system of claim 102 is queried remotely to collect information on results.

105. A diagnostic tool for detecting a target agent in a sample, comprising:
(a) a capture moiety which binds preferentially to a target agent to form a target agent-capture moiety complex;
(b) a tracking component that can be associated with said target agent-capture moiety complex; and
(c) a detection device.

106. A method of doing business, said method comprising use of a signal related to a presence of a tracking component to determine appropriate medical intervention for a patient, said method comprising:
(a) obtaining a sample from said patient whereby a target agent may be present in said sample;
(b) mixing said sample with tracking complexes comprising said tracking component conjugated to a capture moiety specific for said target agent, thereby producing a first mixture comprising reacted complexes, which are those of said tracking complexes that are associated with said target agent, and unreacted complexes, which are those of said tracking complexes that are not associated with said target agent;
(c) contacting said first mixture with immobilized, binding partners, wherein said immobilized binding partners facilitate separation of said unreacted complexes from said reacted complexes to produce a second mixture comprising said unreacted complexes and a third mixture comprising said reacted complexes
(d) providing a detection device comprising a reader, wherein said reader detects said signal based on a presence of said tracking component;
(e) operating said detection device to interrogate said third mixture; and
(f) detecting said signal, wherein said signal is indicative of said presence of said tracking component in said third mixture, which is indicative of said presence of said target agent in said sample.

107. A radio frequency signal used to determine appropriate medical intervention for a patient, whereby said radio frequency signal is indicative of a presence of a target agent in a sample taken from said patient.

108. A method of determining a presence of a target agent in a sample wherein said target agent is not contacted with a detection device, comprising:
(a) mixing said sample with tracking complexes comprising a tracking component conjugated to a capture moiety specific for said target agent, thereby producing a first mixture comprising reacted complexes, which are those of said tracking complexes that are associated with said target agent, and unreacted complexes, which are those of said tracking complexes that are not associated with said target agent;
(b) separating said target agent from said tracking components in said reacted complexes to produce a second mixture comprising said target agent and a third mixture comprising said tracking components;
(c) providing a detection device comprising a reader, wherein said reader detects a signal based on a presence of said tracking component;
(d) operating said detection device to interrogate said third mixture; and
(e) detecting said signal, wherein said signal is indicative of said presence of said tracking component in said third mixture, which is indicative of said presence of said target agent in said sample.

109. The method of claim 108, wherein said target agent is a nucleic acid and said capture moiety is a first capture oligo, and further wherein said first mixture further comprises reactive group complexes comprising a reactive group conjugated to a second capture oligo, and further wherein said reacted complexes comprise those of said tracking complexes that are associated with said target agent further complexed with said reactive group complexes; and further wherein said reacted complexes are subjected to a ligation and a denaturation prior to being contacted with immobilized binding partners that specifically associate with said reactive group.

110. The method of claim 109, wherein said second mixture is a solution phase and said third mixture is an immobilized phase, and further wherein said separating of step (b) comprises removing said solution phase from said immobilized phase.

111. The method of claim 109, wherein said immobilized binding partners are immobilized on a matrix to be subjected to interrogation by said detection device.

112. The method of claim 108, further comprising after step (a) and prior to step (b):
(a) contacting said first mixture with immobilized binding partners, wherein said immobilized binding partners facilitate separation of said unreacted complexes from said reacted complexes to produce a solution phase comprising said unreacted complexes and an immobilized phase comprising said reacted complexes; and
(b) removing said solution phase while retaining said immobilized phase.

113. The method of claim 112, wherein said separating of (b) comprises releasing said tracking components into a liquid phase, wherein said liquid phase is said third mixture.

114. The method of claim 112, wherein said separating of (b) comprises releasing said target agents into a liquid phase and removing said liquid phase from said immobilized phase; wherein said immobilized phase subsequent to release of said target agents into said liquid phase is said third mixture.

115. The method of claim 108, further comprising determining a presence of a plurality of target agents in a sample, wherein said capture moiety comprises a plurality of capture moieties, each of which is specific for only one of said plurality of target agents, wherein said mixing results in the creation of a set of reacted complexes, wherein each of said set links a given target agent of said plurality of target agents to a given capture moiety of said plurality of capture moieties such that no other of said reacted complexes comprises said given target agent with a different capture moiety than said given capture moiety, and no other of said reacted complexes comprises said given capture moiety with a different target agent than said given target agent.

116. The method of claim 115, wherein said tracking component comprises a plurality of tracking components, and further wherein each of said set of reacted complexes links a given tracking component to said given target agent and said given capture moiety such that no other of said reacted complexes comprises said given tracking component with a different target agent than said given target agent or a different capture moiety than said given capture moiety, and no other of said reacted complexes comprises said given target agent and said given capture moiety with a different tracking component than said given tracking component.

117. A method of determining a presence of a target nucleic acid in a sample comprising:
(a) mixing a first complex comprising a tracking component capable of generating a signal, and a first nucleic acid with a sample suspected of containing a target nucleic acid capable of forming a nucleic acid duplex with said first nucleic acid to form a second complex;
(b) contacting said second complex with a moiety capable of associating with said duplex, wherein said moiety is capable of effecting said signal; and
(c) determining said presence of said target nucleic acid by detecting an effect imparted by said moiety on said signal.

118. The method of claim 117, wherein said moiety is at least one member of the group consisting of nucleic acid-binding proteins, intercalating agents, metallo complexes, cis-platin, heme compounds, ruthenium-containing compounds, platinum-containing compounds, iron-containing compounds, and transition metal-containing compounds.

119. The method of claim 117, wherein said effect comprises enabling said tracking component to display said signal.

120. The method of claim 117, wherein said effect comprises enabling said tracking component to alter said signal relative to a baseline signal generated in the absence of said moiety.

121. The method of claim 120, wherein said effect comprises enabling said tracking component to increase said signal relative to a baseline signal generated in the absence of said moiety.

122. The method of claim 120, wherein said effect comprises enabling said tracking component to enhance said signal relative to a baseline signal generated in the absence of said moiety.

123. The method of claim 120, wherein said effect comprises enabling said tracking component to diminish said signal relative to a baseline signal generated in the absence of said moiety.

124. The method of claim 120, wherein said effect comprises enabling said tracking component to alter a frequency of said signal relative to a baseline signal generated in the absence of said moiety.

125. The method of claim 120, wherein said effect comprises enabling said tracking component to alter a wavelength of said signal relative to a baseline signal generated in the absence of said moiety.

126. The method of claim 117, wherein said first complex is immobilized to a matrix.

127. A method of determining a sequence of a target nucleic acid comprising:
(a) mixing a first complex comprising a tracking component capable of generating a signal, and a first nucleic acid with a sample suspected of containing a target nucleic acid capable of forming a nucleic acid duplex with said first nucleic acid to form a second complex;
(b) contacting said second complex with a moiety capable of associating with said duplex, wherein said moiety is capable of effecting said signal; and
(c) determining said sequence of said target nucleic acid by detecting an effect imparted by said moiety on said signal.

128. The method of claim 127, wherein said moiety is at least one member of the group consisting of: nucleic acid-binding proteins, intercalating agents, metallo complexes, cis-platin, heme compounds, ruthenium-containing compounds, platinum-containing compounds, iron-containing compounds, and transition metal-containing compounds.

129. The method of claim 127, wherein said effect comprises enabling said tracking component to display said signal.

130. The method of claim 127, wherein said effect comprises enabling said tracking component to alter said signal relative to a baseline signal generated in the absence of said moiety.

131. The method of claim 130, wherein said effect comprises enabling said tracking component to increase said signal relative to a baseline signal generated in the absence of said moiety.

132. The method of claim 130, wherein said effect comprises enabling said tracking component to enhance said signal relative to a baseline signal generated in the absence of said moiety.

133. The method of claim 130, wherein said effect comprises enabling said tracking component to diminish said signal relative to a baseline signal generated in the absence of said moiety.

134. The method of claim 130, wherein said effect comprises enabling said tracking component to alter a frequency of said signal relative to a baseline signal generated in the absence of said moiety.

135. The method of claim 130, wherein said effect comprises enabling said tracking component to alter a wavelength of said signal relative to a baseline signal generated in the absence of said moiety.

136. The method of claim 127, wherein said first complex is immobilized to a matrix.

137. A method of genotyping a SNP, said method comprising:
(a) mixing a first complex comprising a tracking component capable of generating a signal, and a first nucleic acid with a sample suspected of containing a second nucleic acid capable of forming a nucleic acid duplex with said first nucleic acid to form a second complex, wherein said second nucleic acid comprises said SNP;
(b) contacting said second complex with a moiety capable of associating with said duplex, wherein said moiety is capable of effecting said signal; and
(c) genotyping said SNP by detecting an effect imparted by said moiety on said signal.

138. The method of claim 137, wherein said moiety is at least one member of the group consisting of nucleic acid-binding proteins, intercalating agents, metallo complexes, cis-platin, heme compounds, ruthenium-containing compounds, platinum-containing compounds, iron-containing compounds, and transition metal-containing compounds.

139. The method of claim 137, wherein said effect comprises enabling said tracking component to display said signal.

140. The method of claim 137, wherein said effect comprises enabling said tracking component to alter said signal relative to a baseline signal generated in the absence of said moiety.

141. The method of claim 140, wherein said effect comprises enabling said tracking component to increase said signal relative to a baseline signal generated in the absence of said moiety.

142. The method of claim 140, wherein said effect comprises enabling said tracking component to enhance said signal relative to a baseline signal generated in the absence of said moiety.

143. The method of claim 140, wherein said effect comprises enabling said tracking component to diminish said signal relative to a baseline signal generated in the absence of said moiety.

144. The method of claim 140, wherein said effect comprises enabling said tracking component to alter a frequency of said signal relative to a baseline signal generated in the absence of said moiety.

145. The method of claim 140, wherein said effect comprises enabling said tracking component to alter a wavelength of said signal relative to a baseline signal generated in the absence of said moiety.

146. The method of claim 137, wherein said first complex is immobilized to a matrix.

147. A method of detecting a nucleic acid mutation, said method comprising:
(a) mixing a first complex comprising a tracking component capable of generating a signal, and a first nucleic acid with a sample suspected of containing a second nucleic acid capable of forming a nucleic acid duplex with said first nucleic acid to form a second complex;
(b) contacting said second complex with a moiety capable of associating with said duplex, wherein said moiety is capable of effecting said signal, wherein said second nucleic acid comprises said nucleic acid mutation; and
(c) detecting said nucleic acid mutation by detecting an effect imparted by said moiety on said signal.

148. The method of claim 147, wherein said moiety is at least one member of the group consisting of: nucleic acid-binding proteins, intercalating agents, metallo complexes, cis-platin, heme compounds, ruthenium-containing compounds, platinum-containing compounds, iron-containing compounds, and transition metal-containing compounds.

149. The method of claim 147, wherein said effect comprises enabling said tracking component to display said signal.

150. The method of claim 147, wherein said effect comprises enabling said tracking component to alter said signal relative to a baseline signal generated in the absence of said moiety.

151. The method of claim 150, wherein said effect comprises enabling said tracking component to increase said signal relative to a baseline signal generated in the absence of said moiety.

152. The method of claim 150, wherein said effect comprises enabling said tracking component to enhance said signal relative to a baseline signal generated in the absence of said moiety.

153. The method of claim 150, wherein said effect comprises enabling said tracking component to diminish said signal relative to a baseline signal generated in the absence of said moiety.

154. The method of claim 150, wherein said effect comprises enabling said tracking component to alter a frequency of said signal relative to a baseline signal generated in the absence of said moiety.

155. The method of claim 150, wherein said effect comprises enabling said tracking component to alter a wavelength of said signal relative to a baseline signal generated in the absence of said moiety.

156. The method of claim 147, wherein said first complex is immobilized to a matrix.

157. An in vivo method of determining the presence of a target agent, comprising:
(a) administering a first complex comprising a tracking component capable of generating a signal, and a binding moiety capable of associating with said target agent, to a patient in a clinically-effective amount;
(b) scanning said patient with a reader capable of detecting said signal; and
(c) detecting said signal.

158. The method of claim 157, wherein said binding moiety is at least one member of the group consisting of: antibodies, antigens, proteins, ligands, nucleic acids, receptors, toxins, immunoglobulins, metabolites, hormones and receptor binding agents.

159. The method of claim 158, wherein the binding moiety is capable of binding a cancer marker.

160. The method of claim 158, wherein the binding moiety is capable of binding a genetic mutation.

161. The method of claim 158, wherein the binding moiety is capable of binding nucleic acid sequence.

162. The method of claim 158, wherein the binding moiety is capable of binding a protein.

163. The method of claim 158, wherein the binding moiety is capable of binding a metabolite.

164. The method of claim 158, wherein the binding moiety is capable of binding a toxin.

165. The method of claim 158, wherein the binding moiety is capable of binding a drug.

166. The method of claim 158, wherein the binding moiety is capable of binding a pathogen.

167. The method of claim 158, wherein the binding moiety is capable of binding a microorganism.

168. The method of claim 158, wherein the binding moiety is capable of binding a virus.

169. The method of claim 157, wherein said tracking component is an RFID device.

170. The method of claim 169, further comprising the steps of:
(a) interrogating said RFID device with a reader capable of generating a response signal from said RFID device of sufficient energy to destroy a cell associated with said target agent.

171. The method of claim 170, wherein said energy is equivalent to 0.25-10 gray.

172. The method of claim 170, wherein said cell is a cancer cell.

173. The method of claim 170, wherein said cell is a microorganism.

174. The method of claim 170, wherein said cell is a pathogen.

175. The method of claim 170, wherein said cell is a virally-infected cell.

176. A composition comprising a tracking component, a biomolecule, and a metal-containing compound, wherein said metal-containing compound is a component of an antenna that affects a signal between said tracking component and a reader.

177. The composition of claim 176, wherein said antenna enables detection of said tracking component by said reader.

178. The composition of claim 176, wherein said metal-containing compound alters at least one characteristic of said signal.

179. The composition of claim 178, wherein said characteristic is at least one of the group consisting of strength, frequency, and wavelength.

180. The composition of claim 176, wherein said biomolecule comprises at least one of the group consisting of a DNA, a protein, and a cyclic organic compound.

181. The composition of claim 180, wherein said DNA is a double-stranded DNA.

182. The composition of claim 180, wherein said cyclic organic compound is porphyrin.

183. The composition of claim 176, wherein an association between said metal-containing compound and said biomolecule is selected from the group consisting of intercalation, complexation, minor groove binding, minor groove association, major groove binding, major groove intercalation, covalent interaction, and noncovalent interaction.

184. The composition of claim 183, wherein said metal-containing compound is an intercalating agent selected from the group comprising: ferritin, ethidiumcis-platin, tris(phenanthroline)zinc salt, tris(phenanthroline)ruthenium salt, tris(phenantroline)cobalt salt, di(phenanthroline)zinc salt, di(phenanthroline)ruthenium salt, di(phenanthroline)cobalt salt, bipyridine platinum salt, terpyridine platinum salt, phenanthroline platinum salt, tris(bipyridyl)zinc salt, tris(bipyridyl)ruthenium salt, tris(bipyridyl)cobalt salt, di(bipyridyl)zinc salt, di(bipyridyl)ruthenium salt, and di(bipyridyl)cobalt salt.

185. The composition of claim 176, wherein said metal-containing compound comprises at least one transition metal selected from the group consisting of: cadmium, copper, cobalt, palladium, zinc, iron, ruthenium, rhodium, osmium, rhenium, platinum, scandium, titanium, vanadium, chromium, manganese, nickel, molybdenum, technetium, tungsten, and iridium.

186. The composition of claim 176, wherein said metal-containing compound comprises a transition metal complex that includes at least one ligand selected from the group consisting of sigma donors and pi donors.

187. The composition of claim 176, wherein said metal-containing compound comprises an electroactive marker.

188. The composition of claim 176, wherein said metal-containing compound associates with said biomolecule to form M-DNA.
